SEQUENCE LISTING (92 sequences)

SEQ ID NO: 1 - protein, 9 aa, synthetic construct
  REGION 1..9: note = conditional peptide
  SITE 6: note = conditionally reactive group
STETSXVLY

SEQ ID NO: 2 - protein, 9 aa, synthetic construct
  REGION 1..9: note = conditional peptide
  SITE 7: note = conditionally reactive group
STETSTXLY

SEQ ID NO: 3 - protein, 9 aa, synthetic construct
  REGION 1..9: note = conditional peptide
  SITE 6: note = conditionally reactive group
VTELHXISY

SEQ ID NO: 4 - protein, 9 aa, synthetic construct
  REGION 1..9: note = conditional peptide
  SITE 7: note = conditionally reactive group
VTELHVXSY

SEQ ID NO: 5 - protein, 9 aa, synthetic construct
  REGION 1..9: note = conditional peptide
  SITE 6: note = conditionally reactive group
STDPRXASY

SEQ ID NO: 6 - protein, 9 aa, synthetic construct
  REGION 1..9: note = conditional peptide
  SITE 7: note = conditionally reactive group
STDPRPXSY

SEQ ID NO: 7 - protein, 10 aa, synthetic construct
  REGION 1..10: note = conditional peptide
  SITE 6: note = conditionally reactive group
LLDQGXLNKY

SEQ ID NO: 8 - protein, 10 aa, synthetic construct
  REGION 1..10: note = conditional peptide
  SITE 7: note = conditionally reactive group
LLDQGQXNKY

SEQ ID NO: 9 - protein, 10 aa, synthetic construct
  REGION 1..10: note = conditional peptide
  SITE 8: note = conditionally reactive group
LLDQGQLXKY

SEQ ID NO: 10 - protein, 9 aa, synthetic construct
  REGION 1..9: note = conditional peptide
  SITE 6: note = conditionally reactive group
YLLPAXVHI

SEQ ID NO: 11 - protein, 9 aa, synthetic construct
  REGION 1..9: note = conditional peptide
  SITE 3: note = conditionally reactive group
YLXPAIVHI

SEQ ID NO: 12 - protein, 9 aa, synthetic construct
  REGION 1..9: note = conditional peptide
  SITE 3: note = conditionally reactive group
YAXPAIVHI

SEQ ID NO: 13 - protein, 9 aa, synthetic construct
  REGION 1..9: note = conditional peptide
  SITE 3: note = conditionally reactive group
YMXPAIVHT

SEQ ID NO: 14 - protein, 9 aa, synthetic construct
  REGION 1..9: note = conditional peptide
  SITE 3: note = conditionally reactive group
  SITE 6: note = conditionally reactive group
YAXPAXVHI

SEQ ID NO: 15 - protein, 9 aa, synthetic construct
  REGION 1..9: note = conditional peptide
  SITE 3: note = conditionally reactive group
ILXEHIHKL

SEQ ID NO: 16 - protein, 9 aa, synthetic construct
  REGION 1..9: note = conditional peptide
  SITE 6: note = conditionally reactive group
ILMEHXHKL

SEQ ID NO: 17 - protein, 9 aa, synthetic construct
  REGION 1..9: note = conditional peptide
  SITE 6: note = conditionally reactive group
IAMEHXHKL

SEQ ID NO: 18 - protein, 8 aa, synthetic construct
  REGION 1..8: note = conditional peptide
  SITE 3: note = conditionally reactive group
ILXEIHKL

SEQ ID NO: 19 - protein, 9 aa, synthetic construct
  REGION 1..9: note = conditional peptide
  SITE 8: note = conditionally reactive group
KILGFVFXV

SEQ ID NO: 20 - protein, 9 aa, synthetic construct
  REGION 1..9: note = conditional peptide
  SITE 5: note = conditionally reactive group
RIYRXGATR

SEQ ID NO: 21 - protein, 9 aa, synthetic construct
  REGION 1..9: note = conditional peptide
  SITE 3: note = conditionally reactive group
AIXLTLTTK

SEQ ID NO: 22 - protein, 9 aa, synthetic construct
  REGION 1..9: note = conditional peptide
  SITE 3: note = conditionally reactive group
ALXLTLTTR

SEQ ID NO: 23 - protein, 9 aa, synthetic construct
  REGION 1..9: note = conditional peptide
  SITE 3: note = conditionally reactive group
AIXLTLTTR

SEQ ID NO: 24 - protein, 9 aa, synthetic construct
  REGION 1..9: note = conditional peptide
  SITE 3: note = conditionally reactive group
RLXVGSIPR

SEQ ID NO: 25 - protein, 9 aa, synthetic construct
  REGION 1..9: note = conditional peptide
  SITE 3: note = conditionally reactive group
RIXVGSIPK

SEQ ID NO: 26 - protein, 9 aa, synthetic construct
  REGION 1..9: note = conditional peptide
  SITE 3: note = conditionally reactive group
RIXVGSIPR

SEQ ID NO: 27 - protein, 9 aa, synthetic construct
  REGION 1..9: note = conditional peptide
  SITE 3: note = conditionally reactive group
IIXKGGTSR

SEQ ID NO: 28 - protein, 9 aa, synthetic construct
  REGION 1..9: note = conditional peptide
  SITE 3: note = conditionally reactive group
IAXKGGTSR

SEQ ID NO: 29 - protein, 9 aa, synthetic construct
  REGION 1..9: note = conditional peptide
  SITE 3: note = conditionally reactive group
AYXHMVTHF

SEQ ID NO: 30 - protein, 9 aa, synthetic construct
  REGION 1..9: note = conditional peptide
  SITE 6: note = conditionally reactive group
AYVHMXTHF

SEQ ID NO: 31 - protein, 9 aa, synthetic construct
  REGION 1..9: note = conditional peptide
  SITE 7: note = conditionally reactive group
AYVHMVXHF

SEQ ID NO: 32 - protein, 9 aa, synthetic construct
  REGION 1..9: note = conditional peptide
  SITE 3: note = conditionally reactive group
ATXHMVTHF

SEQ ID NO: 33 - protein, 9 aa, synthetic construct
  REGION 1..9: note = conditional peptide
  SITE 3: note = conditionally reactive group
AFXHMVTHF

SEQ ID NO: 34 - protein, 9 aa, synthetic construct
  REGION 1..9: note = conditional peptide
  SITE 3: note = conditionally reactive group
  SITE 7: note = conditionally reactive group
AFXHMVXHF

SEQ ID NO: 35 - protein, 9 aa, synthetic construct
  REGION 1..9: note = conditional peptide
  SITE 4: note = conditionally reactive group
KYPXNFFLL

SEQ ID NO: 36 - protein, 9 aa, synthetic construct
  REGION 1..9: note = conditional peptide
  SITE 4: note = conditionally reactive group
  SITE 7: note = conditionally reactive group
KYPXNFXLL

SEQ ID NO: 37 - protein, 9 aa, synthetic construct
  REGION 1..9: note = conditional peptide
  SITE 6: note = conditionally reactive group
KYPENXFLL

SEQ ID NO: 38 - protein, 9 aa, synthetic construct
  REGION 1..9: note = conditional peptide
  SITE 4: note = conditionally reactive group
  SITE 7: note = conditionally reactive group
KFPXNFXLL

SEQ ID NO: 39 - protein, 9 aa, synthetic construct
  REGION 1..9: note = conditional peptide
  SITE 5: note = conditionally reactive group
AARGXTLAM

SEQ ID NO: 40 - protein, 10 aa, synthetic construct
  REGION 1..10: note = conditional peptide
  SITE 5: note = conditionally reactive group
AARTXALTAL

SEQ ID NO: 41 - protein, 10 aa, synthetic construct
  REGION 1..10: note = conditional peptide
  SITE 5: note = conditionally reactive group
AARTXALTAM

SEQ ID NO: 42 - protein, 10 aa, synthetic construct
  REGION 1..10: note = conditional peptide
  SITE 9: note = conditionally reactive group
AARTVALTXM

SEQ ID NO: 43 - protein, 9 aa, synthetic construct
  REGION 1..9: note = conditional peptide
  SITE 7: note = conditionally reactive group
AARQPGXMA

SEQ ID NO: 44 - protein, 9 aa, synthetic construct
  REGION 1..9: note = conditional peptide
  SITE 7: note = conditionally reactive group
APRQPGXMA

SEQ ID NO: 45 - protein, 9 aa, synthetic construct
  REGION 1..9: note = conditional peptide
  SITE 8: note = conditionally reactive group
AARQPGLXA

SEQ ID NO: 46 - protein, 9 aa, synthetic construct
  REGION 1..9: note = conditional peptide
  SITE 7: note = conditionally reactive group
SARPPLXSV

SEQ ID NO: 47 - protein, 9 aa, synthetic construct
  REGION 1..9: note = conditional peptide
  SITE 6: note = conditionally reactive group
SARPPXISV

SEQ ID NO: 48 - protein, 9 aa, synthetic construct
  REGION 1..9: note = conditional peptide
  SITE 7: note = conditionally reactive group
SARPPLXSM

SEQ ID NO: 49 - protein, 9 aa, synthetic construct
  REGION 1..9: note = conditional peptide
  SITE 7: note = conditionally reactive group
TPKEKAXAL

SEQ ID NO: 50 - protein, 9 aa, synthetic construct
  REGION 1..9: note = conditional peptide
  SITE 8: note = conditionally reactive group
TPKEKAQXL

SEQ ID NO: 51 - protein, 9 aa, synthetic construct
  REGION 1..9: note = conditional peptide
  SITE 7: note = conditionally reactive group
DLERKVXSL

SEQ ID NO: 52 - protein, 9 aa, synthetic construct
  REGION 1..9: note = conditional peptide
  SITE 8: note = conditionally reactive group
DLERKVEXL

SEQ ID NO: 53 - protein, 8 aa, synthetic construct
  REGION 1..8: note = conditional peptide
  SITE 7: note = conditionally reactive group
DAVVKHXL

SEQ ID NO: 54 - protein, 9 aa, synthetic construct
  REGION 1..9: note = conditional peptide
  SITE 7: note = conditionally reactive group
DLNEKAXAL

SEQ ID NO: 55 - protein, 9 aa, synthetic construct
  REGION 1..9: note = conditional peptide
  SITE 8: note = conditionally reactive group
DLNEKARXL

SEQ ID NO: 56 - protein, 9 aa, synthetic construct
  REGION 1..9: note = conditional peptide
  SITE 7: note = conditionally reactive group
FGLARAXSL

SEQ ID NO: 57 - protein, 9 aa, synthetic construct
  REGION 1..9: note = conditional peptide
  SITE 7: note = conditionally reactive group
FLRGRAXGL

SEQ ID NO: 58 - protein, 9 aa, synthetic construct
  REGION 1..9: note = conditional peptide
  SITE 7: note = conditionally reactive group
NIKKKQXAL

SEQ ID NO: 59 - protein, 9 aa, synthetic construct
  REGION 1..9: note = conditional peptide
  SITE 7: note = conditionally reactive group
TVKQKYXSF

SEQ ID NO: 60 - protein, 10 aa, synthetic construct
  REGION 1..10: note = conditional peptide
  SITE 7: note = conditionally reactive group
AEIDARXDSF

SEQ ID NO: 61 - protein, 10 aa, synthetic construct
  REGION 1..10: note = conditional peptide
  SITE 8: note = conditionally reactive group
AEIDARNXSF

SEQ ID NO: 62 - protein, 10 aa, synthetic construct
  REGION 1..10: note = conditional peptide
  SITE 9: note = conditionally reactive group
AEIDARNDXF

SEQ ID NO: 63 - protein, 9 aa, synthetic construct
  REGION 1..9: note = conditional peptide
  SITE 7: note = conditionally reactive group
NENSLFXSL

SEQ ID NO: 64 - protein, 9 aa, synthetic construct
  REGION 1..9: note = conditional peptide
  SITE 8: note = conditionally reactive group
NENSLFKXL

SEQ ID NO: 65 - protein, 9 aa, synthetic construct
  REGION 1..9: note = conditional peptide
  SITE 7: note = conditionally reactive group
AESIVVXTY

SEQ ID NO: 66 - protein, 9 aa, synthetic construct
  REGION 1..9: note = conditional peptide
  SITE 8: note = conditionally reactive group
AESIVVHXY

SEQ ID NO: 67 - protein, 9 aa, Homo sapiens
TPKEKAQAL

SEQ ID NO: 68 - protein, 9 aa, synthetic construct
  REGION 1..9: note = viral
FLRGRAYGL

SEQ ID NO: 69 - protein, 9 aa, synthetic construct
  REGION 1..9: note = viral
GILGFVFTL

SEQ ID NO: 70 - protein, 9 aa, Homo sapiens
STETSTVLY

SEQ ID NO: 71 - protein, 9 aa, Homo sapiens
NIKKKQEAL

SEQ ID NO: 72 - protein, 9 aa, Homo sapiens
TVKQKYLSF

SEQ ID NO: 73 - protein, 9 aa, Homo sapiens
DLNEKARAL

SEQ ID NO: 74 - protein, 9 aa, Homo sapiens
IIYKGGTSR

SEQ ID NO: 75 - protein, 9 aa, Homo sapiens
FGLARAFSL

SEQ ID NO: 76 - protein, 9 aa, Homo sapiens
SPRPPLISV

SEQ ID NO: 77 - protein, 9 aa, Homo sapiens
ILMEHIHKL

SEQ ID NO: 78 - protein, 9 aa, Homo sapiens
DLERKVESL

SEQ ID NO: 79 - protein, 10 aa, Homo sapiens
AEIDARNDSF

SEQ ID NO: 80 - protein, 10 aa, Homo sapiens
APRTVALTAL

SEQ ID NO: 81 - protein, 9 aa, Homo sapiens
NENSLFKSL

SEQ ID NO: 82 - protein, 9 aa, Homo sapiens
ALFLTLTTK

SEQ ID NO: 83 - protein, 9 aa, Homo sapiens
STDPRPASY

SEQ ID NO: 84 - protein, 9 aa, Homo sapiens
VTELHVISY

SEQ ID NO: 85 - protein, 8 aa, Homo sapiens
DAVVKHVL

SEQ ID NO: 86 - protein, 9 aa, Homo sapiens
AYVHMVTHF

SEQ ID NO: 87 - protein, 9 aa, Homo sapiens
AESIVVHTY

SEQ ID NO: 88 - protein, 10 aa, Homo sapiens
LLDQGQLNKY

SEQ ID NO: 89 - protein, 9 aa, Homo sapiens
RLFVGSIPK

SEQ ID NO: 90 - protein, 9 aa, Homo sapiens
APRQPGLMA

SEQ ID NO: 91 - protein, 9 aa, Homo sapiens
YLLPAIVHI

SEQ ID NO: 92 - protein, 9 aa, Homo sapiens
KYPENFFLL

(19) United States
(12) Patent Application Publication
(10) Pub. No.: US 2024/0358831 A1
(43) Pub. Date: Oct. 31, 2024

(54) SELECTION OF IMMUNE CELLS USING PEPTIDE MHC COMPLEXES GENERATED BY CONDITIONAL LIGAND EXCHANGE

(71) Applicants: IMMATICS US, INC., Houston, TX (US); Immatics Biotechnologies GmbH, Tuebingen (DE)

(72) Inventors: Stefanie SPALT, Tuebingen (DE); Claudia WAGNER, Tuebingen (DE); Dominik MAURER, Tuebingen (DE); Heiko SCHUSTER, Tuebingen (DE); Steffen WALTER, Houston, TX (US)

(21) Appl. No.: 18/685,736

(22) PCT Filed: Aug. 24, 2022
(86) PCT No.: PCT/EP2022/073595

§ 371 Date: Feb. 22, 2024

Related U.S. Application Data
(60) Provisional application No. 63/236,558, filed on Aug. 24, 2021.

(30) Foreign Application Priority Data
Aug. 24, 2021 (EP) 21192884.1

(51) Int. Cl. A61K 39/00; C07K 14/74; C12N 5/0783

(52) U.S. Cl. CPC A61K 39/4632; A61K 39/4611; C07K 14/70539; C12N 5/0636; C12N 2502/11

(57) ABSTRACT

The present invention relates to a method for selecting an immune cell expressing on its surface an antigen-binding protein specifically binding to a complex of a peptide A (PA) and a Major Histocompatibility Complex (MHC) molecule, comprising the steps of (i) providing a plurality of immune cells expressing different antigen-binding proteins; (ii) contacting the plurality of immune cells with a first composition comprising a complex 1A, comprising a MHC molecule 1 (M1) and a peptide A (PA), and a complex 1X, comprising M1 and a peptide B (PB); (iii) contacting the plurality of immune cells with a second composition comprising a complex 2A, comprising a MHC molecule 2 (M2) and PA, and a complex 2X, comprising M2 and a peptide C (PC); (iv) selecting from the plurality of immune cells a cell expressing an antigen binding protein that specifically binds to complex 1A, wherein complexes 1X and 2X, but not 1A and 2A, dissociate upon stimulation with a defined chemical or physical stimulus; and wherein the amino acid sequences of PB and PC differ in at least one amino acid. The invention further relates to an immune cell selected by the method according to the invention, a method of treatment using said immune cell, a kit for selecting a cell expressing on its surface an antigen-binding protein, and a peptide suitable for use in the method according to the invention.

The present invention relates to a method for selecting an immune cell expressing on its surface an antigen-binding protein specifically binding to a complex of a peptide and a MHC molecule (pMHC complex), the method comprising subsequent contacting of the immune cell with a first and a second pMHC complex generated by conditional ligand exchange from pMHC complexes comprising different conditional ligands. The invention further relates to an immune cell selected by the method according to the invention, a method of treatment using said immune cell, a kit for selecting a cell expressing on its surface an antigen-binding protein, and a conditional peptide suitable for use in the method according to the invention.

### BACKGROUND OF THE INVENTION

T cell receptor (TCR) based immunotherapies have become one of the most promising and innovative approaches to treat cancer, viral infections and other immune-modulated disease. Many key steps in the development and implementation of TCR-based therapies require the generation of peptide-MHC (pMHC) complexes. MHC molecules are instable in the absence of a peptide ligand, which is why the generation of each individual pMHC complex requires time-consuming in vitro refolding and purification steps that are incompatible with high throughput applications. Recently, conditional peptide ligands for MHC molecules, which degrade upon exposure to a defined stimulus, were developed. By addition of a peptide of interest the conditional ligand can be replaced. This conditional ligand exchange allows fast and easy production of numerous different pMHC complexes for various applications in the field of T cell identification and characterization.

The invention provides weakly immunogenic conditional peptides having a sequence selected from the group consisting of SEQ ID NO: 1-19, 21-38 and 40-66. These conditional peptides provide inter alia for one or more of the following advantages: (i) no or reduced stimulation/expansion of immune cells specific for conditional peptide:MHC complexes due to low immunogenicity, (ii) strong binding between conditional peptide and MHC molecule, (iii) high refolding yield of conditional peptide:MHC complexes, (iv) stable conditional peptide:MHC complexes in absence of the defined stimulus, (v) low aggregation rate of conditional peptide:MHC complexes, (vi) low degradation rate of conditional peptide:MHC complexes, and (vii) high dissociation rate of the peptide from the MHC molecule upon the defined stimulus.

Furthermore, the invention provides an innovative approach for selecting an immune cell expressing an antigen-binding protein using conditional ligand exchange. By providing this approach, the inventors have overcome several problems of the prior art. The method for selecting an immune cell according to the present invention allows for the use of pMHC complexes generated by conditional ligand exchange, wherein the risk of selecting false positive immune cells is significantly reduced or eliminated. Thus, the method provides inter alia for one or more of the following advantages: the method is (i) less expensive, (ii) faster, (iii) easier, (iv) suitable for high throughput applications, and (v) more accurate.

### SUMMARY OF THE INVENTION

A first aspect of the invention relates to a method for selecting an immune cell expressing on its surface an antigen-binding protein specifically binding to a complex of a peptide A (PA) and a Major Histocompatibility Complex (MHC) molecule, comprising the following steps:
(i) providing a plurality of immune cells expressing different antigen-binding proteins;
(ii) contacting the plurality of immune cells with a first composition comprising:
   (a) a complex 1A, comprising a MHC molecule 1 (M1) and a peptide A (PA), and
   (b) a complex 1X, comprising M1 and a peptide B (PB);
(iii) contacting the plurality of immune cells with a second composition comprising:
   (a) a complex 2A, comprising a MHC molecule 2 (M2), and PA, and
   (b) a complex 2X, comprising M2 and a peptide C (PC);
(iv) selecting from the plurality of immune cells a cell expressing an antigen binding protein that specifically binds to complex 1A;
wherein complexes 1X and 2X, but not 1A and 2A, dissociate upon stimulation with a defined stimulus; and wherein the amino acid sequences of PB and PC differ in at least one amino acid.

A second aspect of the invention relates to a conditional peptide having an amino acid sequence according to any one of SEQ ID NO: 1-19, 21-38 and 40-66, wherein in each sequence the X represents a conditionally reactive group.

A third aspect of the invention relates to an immune cell selected by the method of the first aspect of the invention.

A fourth aspect of the invention relates to a method for determining the sequence of a nucleic acid encoding an antigen-binding protein specifically binding to a pMHC complex 1A comprising the steps of:
(i) isolating a nucleic acid encoding the antigen-binding protein from the immune cell selected in the method of the first aspect of the invention; and
(ii) determining the sequence of the nucleic acid.

A fifth aspect of the invention relates to a method for producing a host cell expressing an antigen-binding protein comprising the steps of:
(i) providing a host cell;
(ii) isolating a nucleic acid encoding the antigen-binding protein from the immune cell selected in the method of the first aspect of the invention;
(iii) producing a genetic construct comprising the nucleic acid isolated in step (ii) or a part of said nucleic acid encoding the antigen-binding protein or an antigen-binding fragment thereof; and
(iv) introducing the genetic construct into the host cell.

A sixth aspect of the invention relates to a method for producing an antigen-binding protein, comprising providing a host cell produced by the method of the fifth aspect of the invention and expressing the genetic construct introduced into said host cell.

A seventh aspect of the invention relates to an antigen binding protein produced by the method of the sixth aspect of the invention.

An eighth aspect of the invention relates to a nucleic acid encoding the antigen binding protein of the seventh aspect of the invention or a vector comprising said nucleic acid.

An ninth aspect of the invention relates to a kit as defined herein below. In particular aspects, the kit is for selecting a cell expressing on its surface an antigen-binding protein specifically binding to a complex of a peptide and a MHC molecule. Said kit comprises:
a) a complex 1X, comprising a peptide B (PB) and a MHC molecule 1 (M1); and a complex 2X, comprising a peptide C (PC) and a MHC molecule 2 (M2); or
b) a peptide (PB) and a peptide (PC) and optionally (an) MHC molecule(s) to which PB and PC bind, in particular M1 and/or M2, thereby forming a complex 1X and 2X, respectively; and
c) optionally β2-microglobulin (02M);
   wherein complexes 1X and 2X, dissociate upon stimulation with a defined stimulus; and wherein the amino acid sequences of PB and PC differ in at least one amino acid.

A tenth aspect of the invention relates to a pharmaceutical composition comprising an immune cell selected by the method of the first aspect of the invention, a host cell produced by the method of the fifth aspect of the invention, an antigen binding protein of the seventh aspect of the invention, and/or a nucleic acid or vector of the eighth aspect of the invention.

An eleventh aspect of the invention relates to an immune cell selected by the method of the first aspect of the invention, a host cell produced by the method of the fifth aspect of the invention, an antigen binding protein of the seventh aspect of the invention, and/or a nucleic acid or vector of the eighth aspect of the invention for use in medicine.

A twelfth aspect of the invention relates to an immune cell selected by the method of the first aspect of the invention, a host cell produced by the method of the fifth aspect of the invention, an antigen binding protein of the seventh aspect of the invention, and/or a nucleic acid or vector of the eighth aspect of the invention for use in a method of diagnosis, treatment or prevention of a neoplastic disease.

A thirteenth aspect of the invention relates to a use of a peptide according to the second aspect of the invention for the preparation of pMHC complexes.

### BRIEF DESCRIPTION OF DRAWINGS

FIG. 1 shows an exemplary process overview. T cells are isolated from blood products (1). Target specific pHLA molecules, produced by, for example, UV mediated peptide exchange (2) together with, for example, anti-CD28 antibody are used to generate, for example, artificial Antigen-presenting Cells (aAPCs) (3). Incubation of T cells with aAPCs in the presence of, for example, soluble cytokines leads to antigenic peptide specific stimulation and thus proliferation (4). pHLAs for the generation of dye-coupled multimers (5) are generated by, for example, a second UV exchange reaction using a different conditional ligand (6). Target specific T cells are detected and/or isolated via pHLA multimer staining (7).

FIG. 2 shows protein yield after UV mediated peptide exchange. Exemplary results of protein yields measured by β2m ELISA after UV exposure of conditional pHLAs in the presence of a rescue peptide (grey) or without rescue peptide (black). Conditional pHLA were A*03_HNRNPR-002, B*07_MUDENG-002 and A*02_DDX5-005. Rescue peptides were HADV-014 (A*03), HIV-007 (B*07) and MLA-001 (A*02), respectively. Mean values of n=4 experiments are shown. Protein yield is shown as % of applied protein concentration. The data suggest that when EC monomers are generated via UV exchange, the UV peptides are not always exchanged completely by the rescue peptides leading to residual amounts of low concentrated UV monomers.

FIG. 3 shows exemplary results of a specific in vitro CD8+ T cell response of a healthy HLA-A*02+ donor towards a conditional ligand. CD8+ T cells were primed using artificial APCs coated with anti-CD28 mAb and HLA-A*02_FLUM-003×MLA-001 generated by UV-mediated ligand exchange of FLUM-003 by MLA-001. After three cycles of stimulation, the detection of peptide-reactive cells was performed by either 2D multimer staining using exchanged pHLA molecules (A) or standard refolded pHLA molecules (B) or 1D staining (C and D). Viable single cells were gated for CD8+ lymphocytes. Frequencies of specific multimer+ cells among CD8+ lymphocytes are indicated. These data show that even low amounts of remaining UV monomers are sufficient to detect cells specific for the conditional ligand by multimer staining.

FIG. 4 shows presence of highly specific T-cell populations recognizing conditional peptide ligands and their parental peptides. CD8+ T cells of HLA matched healthy individuals were primed using artificial APCs coated with anti-CD28 mAb and pHLA molecules. After three cycles of stimulation, the detection of peptide-reactive cells was performed by multimer staining using standard refolded pHLA molecules. 12 wells were tested per condition. Stimulation and readout were either performed with the same peptide or as combination with the related parental peptide or conditional peptide (italic), respectively. Graph A shows results for molecules containing the indicated peptides bound to HLA-A*03, graph B shows results for molecules containing the indicated peptides bound to HLA-B*07.

FIG. 5 shows multimer staining of UV peptide-reactive CD8+ T cells. CD8+ T cells were primed using artificial APCs coated with anti-CD28 mAb and HLA-B*07_MUDENG-002 (A) or HLA-A*03_HNRNPR-002×HADV-14. After three cycles of stimulation, the detection of peptide-reactive cells was performed by multimer staining using standard refolded pHLA molecules (A and B). Viable single cells were gated for CD8+ lymphocytes. Frequencies of specific multimer+ cells among CD8+ lymphocytes are indicated. In both cases, UV peptide-specific T cell populations of 2.4% or 6.7% of CD8+ cells, respectively, could be detected by multimer staining, indicating that also UV peptides derived from human source proteins can stimulate and expand specific T cell populations, even at low concentrations.

FIG. 6 shows detection of false positive T cell populations. CD8+ T cells were primed using artificial APCs coated with anti-CD28 mAb and HLA-A*02_FLUM-003×CMV-001 generated by UV-mediated ligand exchange of FLUM-003 by CMV-001. After three cycles of stimulation, cells were analyzed by three parallel read-out conditions. The detection of peptide-reactive cells was performed by 2D multimer staining using either standard refolded pHLA molecules (Plate A), pHLA molecules generated by UV-mediated ligand exchange of FLUM-003 by CMV-001 or MLA-001 (Plate B) or pHLA molecules generated by UV-mediated ligand exchange of RPL19-005 by CMV-001 or MLA-001 (Plate C). Viable single cells were gated for CD8+ lymphocytes. Frequencies of specific multimer+ cells among CD8+ lymphocytes are indicated. In some cases (see FIG. 6, stimulation position 1 and 2) FLUM-003 specific cells were stimulated and detected when using the same UV monomer for generation of EC monomers for multimer staining (Plate B), whereas using another UV monomer for generation of EC monomers for multimer staining did prevent this unwanted detection of FLUM-003 specific cells (Plate C).

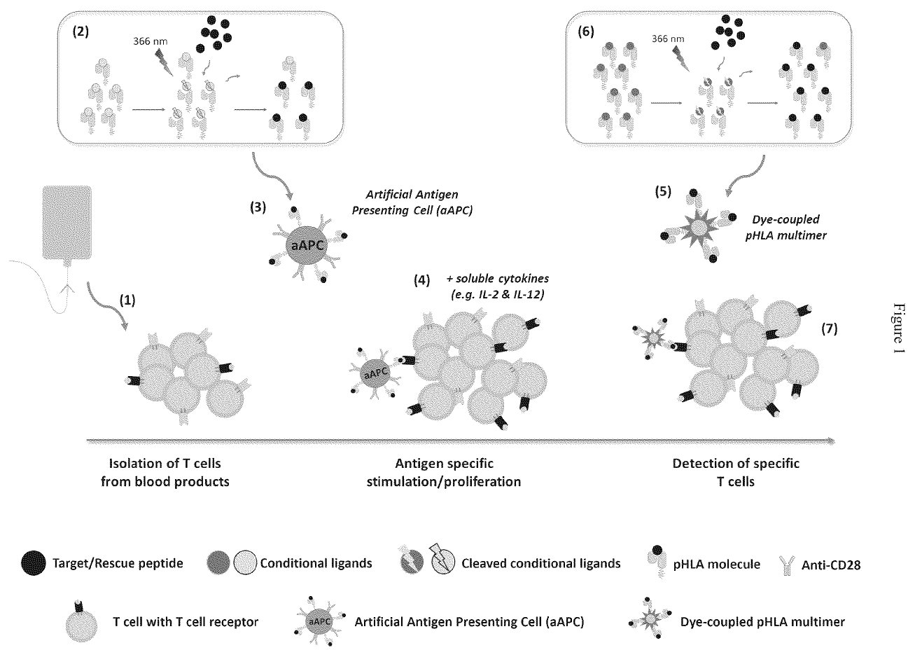

### DETAILED DESCRIPTION OF THE INVENTION

T cell receptor (TCR) based immunotherapies have become one of the most promising and innovative approaches to treat cancer, viral infections and other immune-modulated disease. Many key steps in the development and implementation of TCR-based therapies require the generation of peptide-MHC (pMHC) complexes. MHC molecules are instable in the absence of a peptide ligand, which is why the generation of each individual pMHC complex requires time-consuming in vitro refolding and purification steps that are incompatible with high throughput applications. Recently, conditional peptide ligands for MHC molecules, which degrade upon exposure to a defined stimulus, were developed. By addition of a peptide of interest (or rescue peptide) during this stimulus the conditional ligand can be replaced by this peptide and degradation of the MHC molecule is prevented. This conditional ligand exchange allows fast and easy production of numerous different pMHC complexes for various applications in the field of T cell identification and characterization. A particularly popular conditional ligand exchange is the so-called UV exchange, in which the conditional ligand is cleaved upon UV exposure.

With all the advances that have been made in the field, a TCR identification process with the aim to identify TCRs that specifically bind their target remains a time and cost consuming process. If initially identified TCR have to be deselected during further characterization in individual experiments, this is again very time and cost consuming. Accordingly, further improvements to methods for selecting an immune cell expressing on its surface an antigen-binding protein specifically binding to a complex of a peptide and a MHC molecule are needed.

In contrast to what has previously been reported, the inventors found that there is a significant amount of residual conditional ligand bound to the MHC molecule after, for example, UV exposure. The inventors concluded that if such pMHC complexes comprising residual conditional ligand are used for consecutive steps e.g. stimulation, detection and/or sorting of T cells, this may lead to the stimulation, detection or sorting of T cells specific for pMHC complexes comprising not the peptide of interest, but the conditional ligand instead. Conditional ligands so far disclosed in the art are derived primarily from viral peptides or non-human peptides. Since these peptides are highly immunogenic, the observed problem, i.e. that the identified TCRs may be directed against the conditional ligand itself, is thus particularly pronounced in context of viral and non-human peptides.

To overcome this problem, the inventors have identified new conditional peptide ligands derived from peptides of human origin. Furthermore, those peptides are highly expressed in healthy tissue and are thus less immunogenic, thereby reducing the above indicated problem. The invention provides weakly immunogenic conditional peptides having a sequence selected from the group consisting of SEQ ID NO: 1-19, 21-38 and 40-66. These conditional peptides provide inter alia for one or more of the following advantages: (i) no or reduced stimulation/expansion of immune cells specific for conditional peptide:MHC complexes due to low immunogenicity, (ii) strong binding between conditional peptide and MHC molecule, (iii) high refolding yield of conditional peptide:MHC complexes, (iv) stable conditional peptide:MHC complexes in absence of the defined stimulus, (v) low aggregation rate of conditional peptide:MHC complexes, (vi) low degradation rate of conditional peptide:MHC complexes, and (vii) high dissociation rate of the peptide from the MHC molecule upon the defined stimulus.

To further reduce the identification of false positive TCRs, the inventors have developed an innovative approach for selecting an immune cell expressing an antigen-binding protein with a defined specificity, in which the immune cell is consecutively contacted with two pMHC complexes generated by conditional ligand exchange using two different conditional ligands. In other words, the conditional peptide ligand used for generating a pMHC complex in a first step, in particular a priming or stimulation step, is different from the conditional peptide ligand used to generate the pMHC complex in the second step, in particular an identification or selection step. The inventors demonstrate that this approach significantly reduces the selection of false positive immune cells, i.e. cells that are not specific for pMHC complexes comprising the ligand of interest but instead for pMHC complexes comprising the conditional ligand. By providing this approach, the inventors have overcome problems of the prior art stated above. The method for selecting an immune cell according to the present invention allows for the use of pMHC complexes generated by conditional ligand exchange, wherein the risk of selecting false positive immune cells is significantly reduced or eliminated. Thus, the method provides inter alia for one or more of the following advantages: the method is (i) less expensive, (ii) faster, (iii) easier, (iv) suitable for high throughput applications, and (v) more accurate.

Before the present invention is described in detail below, it is to be understood that this invention is not limited to the particular methodology, protocols and reagents described herein as these may vary. It is also to be understood that the terminology used herein is for the purpose of describing particular embodiments only and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

To practice the present invention, unless otherwise indicated, conventional methods of chemistry, biochemistry, and recombinant DNA techniques are employed which are explained in the literature in the field (cf, e.g., Molecular Cloning: A Laboratory Manual, 2^{nd} Edition, J. Sambrook et al. eds., Cold Spring Harbor Laboratory Press, Cold Spring Harbor 1989).

In the following, some definitions of terms frequently used in this specification to characterize the invention are provided. These terms will, in each instance of its use, in the remainder of the specification have the respectively defined meaning and preferred meanings.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. As used herein, the use of the term “comprising” also discloses the embodiment wherein no features other than the specifically mentioned features are present (i.e. “consisting of”).

As used in this specification and the appended claims, the singular forms “a”, “an”, and “the” include plural referents, unless the content clearly dictates otherwise.

Throughout the instant application, the term “and/or” is a grammatical conjunction that is to be interpreted as encompassing that one or more of the cases it connects may occur. For example, the wording “such native sequence proteins can be prepared using standard recombinant and/or synthetic methods” indicates that native sequence proteins can be prepared using standard recombinant and synthetic methods or native sequence proteins can be prepared using standard recombinant methods or native sequence proteins can be prepared using synthetic methods.

The term “cell” refers in the context of the present invention to eukaryotic cells which contain a nucleus and cell organelles and can be found in protozoa, fungi, plants and animals. Animals can comprise mammalian cells. Mammalian cells comprise inter alia human cells, rodent cells, such as mouse, or rat cells, monkey cells, pig cells or dog cells. Fungi cells inter alia comprise yeast cells. Typical yeast cells used in biotechnology, for example in a yeast surface display, are Saccharomyces cerevisiae cells.

The term “immune cell” refers in the context of this invention to a cell of the immune system. The immune system comprises different cell types such as precursor cells comprising lymphoid stem cells, which ultimately differentiate into B and T lymphocytes and natural killer (NK) cells, and myeloblasts, which ultimately differentiate into granulocytes and monocytes as well as fully differentiated leukocytes. Differentiated leukocytes are thymus-, spleen-, bone marrow or lymph node-derived cells and can be categorized into the main groups of granulocytes, B-lymphocytes, T-lymphocytes and monocytes, macrophages, and mast cells and dendritic cells. Granulocytes are further divided into neutrophil, eosinophil and basophil granulocytes, which phagocytose bacteria, virus or fungi in the blood circulation. B-lymphocytes are precursors of plasma cells and B-memory cells. The group of T cells comprises regulatory T cells, memory T cells, T helper cells and cytotoxic T cells. While T helper cells activate plasma cells and natural killer cells, regulatory T cells inhibit the function of B and other T cells and thus, slow down the immune response. T memory cells are long-living and possess a memory for specific antigens, and cytotoxic T cells recognize and kill tumor cells or cells attacked by viruses by interacting with tumor antigens or antigens of the attacked cells. Examples of T cells and their surface phenotype described by the specific surface markers of the respective T cells are given in below Table 1 (according to Dong and Martinez, Nature Reviews Immunology, 2010):

TABLE 1
Common T cell surface markers (non-exhaustive enumeration).

| Cell: | Surface marker: |
|---|---|
| Cytotoxic T cells: | αβ TCR, CD3, CD8 |
| Regulatory T cells: | αβ TCR, CD3, CD4 |
| Regulatory T cells | αβ TCR, CD3, CD4, CD25, CTLA4, GITR |
| (natural and | |
| inducible): | |
| Natural Killer cells: | NK1.1, SLAMF1, SLAMF6, TGFβ, Vα24, Jα18 |
| T helper cells: | |
| TH1 cells | αβ TCR, CD3, CD4, IL-12R, IFNγR, CXCR3; |
| TH2 cells | αβ TCR, CD3, CD4, IL-4R, IL33R, CCR4, |
| | IL-17RB, CRTH2; |
| TH9 cells | αβ TCR, CD3, CD4; |
| TH17 cells | αβ TCR, CD3, CD4, IL-23R, CCR6, IL-1R, |
| | CD161; |
| TH22 cells | αβ TCR, CD3, CD4, CCR10; |
| TILs | Tumor-infiltrating lymphocytes |
| T memory cells | CCR7 hi, CD44, CD62Lhi, TCR, CD3, |
| | IL-7R (CD127), IL-15R |

The term “antigen-presenting cell” refers to a cell that processes an antigen and displays a complex of an antigenic peptide and a MHC molecule (pMHC complex) on its surface to a T cell. Antigen-presenting cells fall into two categories: professional and non-professional. Professional antigen-presenting cells express MHC-II molecules along with co-stimulatory molecules and pattern recognition receptors. Non-professional antigen-presenting cells express MHC-I molecules.

The term “artificial antigen-presenting cell (aAPC)” refers to a synthetic carrier, e.g. a microparticle or nanoparticle, with attached peptide-MHC complexes and a costimulatory signal (e.g. anti-CD28 antibodies). Microparticles having a similar size as a natural antigen-presenting cell (i.e. having a diameter of approximately 4-20 μm, such as 5 to 10 μm) are preferred. The synthetic carrier may be selected from e.g. poly (glycolic acid), poly(lactic-co-glycolic acid), iron-oxide, liposomes, lipid bilayers, sepharose, polystyrene and polyisocyanopeptides. The synthetic carrier may be coated with streptavidin. In a preferred embodiment, aAPCs are streptavidin-coated polystyrene beads with attached peptide-MHC complexes and anti-CD28 antibodies.

The term “tumor-infiltrating lymphocytes” (TILs) refers in the context of the present invention to T cells and B cells that have migrated towards a tumor and can often be found in the tumor stroma or the tumor itself. TILs typically comprise a cell population of white blood cells that may be used in ACT or autologous cell therapy. Such therapies have already shown promising results, for example in patients with metastatic melanoma in a variety of clinical trials (Guo et al.; “Recent updates on cancer immunotherapy”; Precision Clinical Medicine, 1(2), 2018-65-74). In the context of ACT, TILs are expanded ex vivo from surgically resected tumors or single cell suspensions isolated from tumor fragments. TILs are expanded with a high dose of cytokines, for example IL-2. Selected TIL lines that presented best tumor reactivity are then further expanded in a “rapid expansion protocol” (REP), which uses anti-CD3 activation for a typical period of two weeks. The final post-REP TILs are infused back into the patient. The process can also involve a preliminary chemotherapy regimen to deplete endogenous lymphocytes in order to provide the adoptively transferred TILs with enough access to surround the tumor sites.

The term “immune cell enriched fraction” refers in the context of this invention to a cell population, which is derived from a naturally occurring cell population, e.g. blood, in which the relative abundance of the immune cells has been increased in comparison to their abundance in the naturally occurring cell mixture. One ml of blood of a healthy human subject comprises, e.g. 4.7 to 6.1 million (male), 4.2 to 5.4 million (female) erythrocytes, 4,000-11,000 leukocytes and 200,000-500,000 thrombocytes. Thus, in blood immune cells only constitute 0.06% to 0.25% of the total number of blood cells. An immune cell enriched fraction of blood thus may comprise more than 0.25%, more than 1%, more than 5%, more than 10%, more than 20%, more than 30%, more than 40%, preferably more than 50%, more than 60% more than 70%, even more preferably more than 80%, more than 85% and most preferably more than 90% immune cells. The immune cell enriched fraction may be enriched for one or more subtypes of immune cells. For example, the immune cell enriched fraction may be enriched for lymphoid stem cells, T cells, B cells, plasma cells or combinations. Usually, immune cells in immune cell enriched fractions are selected by using one or more fluorescently labelled antibodies that specifically bind to a surface marker of the immune cells of interest. Suitable surface markers to select T cells or sub-fractions within the group of T cells are indicated in Table 1 above. Cytotoxic T cells can be selected, e.g. by using an antibody that specifically binds to CD8 or by using antibodies that specifically bind to CD8 and CD3.

The term “cell population” refers in the context of this invention to a plurality of cells, which may be homogenous or heterogenous, i.e. a mixture of cells of different characteristic. Blood is an example of a cell population which is a mixture of different cells. Homogenous cell populations can be obtained by selection of a particular subtype or by clonal expansion.

The term “immune cell specific surface marker” refers in the context of this invention to cell surface antigens, which serve as monograms to help identify and classify immune cells. Examples of such markers that characterize different T cell subtypes are indicated in Table 1 above. The majority of immune cell specific surface markers are molecules or antigens within cell's plasma membrane. These molecules serve not only as markers but they also have key functional roles.

The terms “growth factor” or “differentiation factor” are used interchangeably in the context of this invention and refer to molecules that are capable of stimulation cellular growth, cell proliferation and cellular differentiation and regulate multiple cellular processes. Growth factors are usually proteins or steroid hormones. Examples of prevailing molecules are listed in the following (non-exhaustive enumeration): Growth factors, such as colony stimulating factor (CSF), Macrophage colony-stimulating factor (M-CSF), Granulocyte colony-stimulating factor (G-CSF) and Granulocyte macrophage colony-stimulating factor (GM-CSF); epidermal growth factor (EGF); erythropoietin (EPO); fibroblast growth factor (FGF); foetal bovine somatotropin (FBS); hepatocyte growth factor (HGF); insulin; insulin like growth factor (IGF); interleukins; neuregulins; neutrotrophins; T cell growth factor (TCGF); transforming growth factor (TGF); tumor necrosis factor alpha (TNFα); vascular endothelial growth factor (VEGF).

The term “antigen binding protein” refers to a polypeptide or a complex of two or more polypeptides comprising an antigen binding site that is able to specifically bind to an antigenic peptide in a complex with MHC. Examples of antigen binding proteins are antibodies, B cell receptors (BCRs), TCRs, single chain antibodies, single chain TCRs, and chimeric antigen receptors (CAR). As used in the context of the present specification, the term antigen binding protein includes multiple formats, including soluble formats, membrane bound formats, monovalent, bivalent and multivalent formats, monospecifc, bispecific and multispecific formats, single chain formats and formats comprising two or more chains. The term antigen binding protein also includes antigen-binding fragments of an antigen binding protein, e.g. antigen binding fragments of a TCR (see below). In preferred embodiments, the antigen binding protein is a TCR.

The term “T cell receptor” (TCR) refers in the context of this invention to a heterodimeric cell surface protein of the immunoglobulin super-family, which is associated with invariant proteins of the CD3 complex involved in mediating signal transduction. TCRs exist in αβ and γδ forms, which are structurally similar but have quite distinct anatomical locations and probably functions. The extracellular portion of native heterodimeric αβ TCR and γδ TCR each contain two polypeptides, each of which has a membrane-proximal constant domain, and a membrane-distal variable domain. Each of the constant and variable domains include an intra-chain disulfide bond. The variable domains contain the highly polymorphic loops analogous to the complementarity determining regions (CDRs) of antibodies. The use of TCR gene therapy overcomes a number of current hurdles. For example, it allows equipping the subjects' (patients') own T cells with desired specificities and generation of sufficient numbers of T cells in a short period of time, avoiding their exhaustion. In such embodiments, the TCR will be transduced into potent T cells (e.g. central memory T cells or T cells with stem cell characteristics), which may ensure better persistence, preservation and function upon transfer. TCR-engineered T cells will be infused into cancer patients rendered lymphopenic by chemotherapy or irradiation, allowing efficient engraftment but inhibiting immune suppression. Native alpha-beta heterodimeric TCRs have an alpha chain and a beta chain. Each alpha chain comprises variable, joining and constant regions, and the beta chain also usually contains a short diversity region between the variable and joining regions, but this diversity region is often considered as part of the joining region. The constant, or C, regions of TCR alpha and beta chains are referred to as TRAC and TRBC respectively (Lefranc, (2001), Curr Protoc Immunol Appendix 1: Appendix 10). Each variable region, comprises three “complementarity determining regions” (CDRs) embedded in a framework sequence, one being the hypervariable region named CDR3. The alpha chain CDRs are referred to as CDRa1, CDRa2, CDRa3, and the beta chain CDRs are referred to as CDRb1, CDRb2, CDRb3. There are several types of alpha chain variable (Valpha) regions and several types of beta chain variable (Vbeta) regions distinguished by their framework, CDR1 and CDR2 sequences, and by a partly defined CDR3 sequence. The Valpha types are referred to in IMGT nomenclature by a unique TRAV number, Vbeta types are referred in IMGT nomenclature to by a unique TRBV number (Folch and Lefranc, (2000), Exp Clin Immunogenet 17(1): 42-54; Scaviner and Lefranc, (2000), Exp Clin Immunogenet 17(2): 83-96; Lefranc and Lefranc, (2001), “T cell Receptor Factsbook”, Academic Press). For more information on immunoglobulin antibody and TCR genes see the international ImMunoGeneTics Information System®, Lefranc M-P et al (Nucleic Acids Res. 2015 January; 43 (Database issue): D413-22; and http://www.imgt.org/). A conventional TCR antigen-binding site usually includes six CDRs, comprising the CDR set from each of an alpha and a beta chain variable region, wherein CDR1 and CDR3 sequences are relevant to the recognition and binding of the antigenic peptide in a complex with MHC protein and the CDR2 sequences are relevant to the recognition and binding of the MHC protein. Analogous to antibodies, TCRs comprise framework regions which are amino acid sequences interposed between CDRs, i.e. to those portions of TCR alpha and beta chain variable regions that are relatively conserved among different TCRs. The alpha and beta chains of a TCR each have four FRs, herein designated FR1-a, FR2-a, FR3-a, FR4-a, and FR1-b, FR2-b, FR3-b, FR4-b, respectively. Accordingly, the alpha chain variable domain may thus be designated as (FR1-a)-(CDRa1)-(FR2-1)-(CDRa2)-(FR3-a)-(CDRa3)-(FR4-a) and the beta chain variable domain may thus be designated as (FR1-b)-(CDRb1)-(FR2-b)-(CDRb2)-(FR3-b)-(CDRb3)-(FR4-b).

The term “B cell receptor” (BCR) refers to a transmembrane protein complex on the surface of B cells. Structurally, the BCR comprises a membrane-bound immunoglobulin molecule of one isotype (IgD, IgM, IgA, IgG, or IgE) and a signal transduction moiety: An Ig-α/Ig-β(CD79) heterodimer, linked by disulfide bridges. Each member of the dimer spans the plasma membrane and has a cytoplasmic tail bearing an immunoreceptor tyrosine-based activation motif (ITAM). The portion of the BCR that recognizes antigens is made up of three disparate genetic regions, termed V. D, and J, that are spliced and recombined at the genetic level in a combinatorial process unique to the immune system. When a B cell is activated by its first encounter with an antigen that binds to its receptor (its “cognate antigen”), the cell proliferates and differentiates to generate a population of antibody-secreting plasma B cells and memory B cells. The BCR controls B cell activation by biochemical signaling and by physical acquisition of antigens from immune synapses with antigen-presenting cells and has two crucial functions upon interaction with the antigen. One function is signal transduction, involving changes in receptor oligomerization. The second function is to mediate internalization for subsequent processing of the antigen and presentation of peptides to helper T cells.

The term “antibody” in the context of the present invention refers to secreted immunoglobulins which lack the transmembrane region and can thus, be released into the bloodstream and body cavities. Human antibodies are grouped into different isotypes based on the heavy chain they possess. There are five types of human Ig heavy chains denoted by the Greek letters: α, γ, δ, ε, and μ. The type of heavy chain present defines the class of antibody, i.e. these chains are found in IgA, IgD, IgE, IgG, and IgM antibodies, respectively, each performing different roles, and directing the appropriate immune response against different types of antigens. Distinct heavy chains differ in size and composition; and may comprise approximately 450 amino acids (Janeway et al. (2001) Immunobiology, Garland Science). IgA is found in mucosal areas, such as the gut, respiratory tract and urogenital tract, as well as in saliva, tears, and breast milk and prevents colonization by pathogens (Underdown & Schiff (1986) Annu. Rev. Immunol. 4:389-417). IgD mainly functions as an antigen receptor on B cells that have not been exposed to antigens and is involved in activating basophils and masT-cells to produce antimicrobial factors (Geisberger et al. (2006) Immunology 118:429-437; Chen et al. (2009) Nat. Immunol. 10:889-898). IgE is involved in allergic reactions via its binding to allergens triggering the release of histamine from masT-cells and basophils. IgE is also involved in protecting against parasitic worms (Pier et al. (2004) Immunology, Infection, and Immunity, ASM Press). IgG provides the majority of antibody-based immunity against invading pathogens and is the only antibody isotype capable of crossing the placenta to give passive immunity to fetus (Pier et al. (2004) Immunology, Infection, and Immunity, ASM Press). In humans there are four different IgG subclasses (IgG1, 2, 3, and 4), named in order of their abundance in serum with IgG1 being the most abundant (˜66%), followed by IgG2 (˜23%), IgG3 (˜7%) and IgG (˜4%). The biological profile of the different IgG classes is determined by the structure of the respective hinge region. IgM is expressed on the surface of B cells in a monomeric form and in a secreted pentameric form with very high avidity. IgM is involved in eliminating pathogens in the early stages of B cell mediated (humoral) immunity before sufficient IgG is produced (Geisberger et al. (2006) Immunology 118:429-437). Antibodies are not only found as monomers but are also known to form dimers of two Ig units (e.g. IgA), tetramers of four Ig units (e.g. IgM of teleost fish), or pentamers of five Ig units (e.g. mammalian IgM). Antibodies are typically made of four polypeptide chains comprising two identical heavy chains and identical two light chains which are connected via disulfide bonds and resemble a “Y”-shaped macro-molecule. Each of the chains comprises a number of immunoglobulin domains out of which some are constant domains and others are variable domains. Immunoglobulin domains consist of a 2-layer sandwich of between 7 and 9 antiparallel ˜-strands arranged in two ˜-sheets. Typically, the heavy chain of an antibody comprises four Ig domains with three of them being constant (CH domains: CH1, CH2, CH3) domains and one of the being a variable domain (VH). The light chain typically comprises one constant Ig domain (CL) and one variable Ig domain (V L). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. Term antibody as used herein also encompasses a chimeric antibody, a humanized antibody or a human antibody.

The term “chimeric antigen receptor” (CAR; also known as chimeric immunoreceptor, chimeric T cell receptor, artificial T cell receptor) in the context of the present invention refers to engineered receptors, which graft an arbitrary specificity onto an immune effector cell, preferably a T cell. Cells are genetically equipped with a CAR, which is a composite membrane receptor molecule and provides both targeting specificity and T cell activation. The most common form of CARs are fusions of single chain variable fragment (scFv) derived from monoclonal antibodies, fused to CD3 transmembrane- and endodomain. The CAR targets the T cell to a desired cellular target through an antibody-derived binding domain in the extracellular moiety, and T cell activation occurs via the intracellular moiety signalling domains when the target is encountered. The transfer of the coding sequence of these receptors into suitable cells, in particular T cells, is commonly facilitated by retro- or lentiviral vectors. The receptors are called chimeric because they are composed of parts from different sources.

The term “antigen-binding fragment” or “binding fragment” of an antibody, TCR, or BCR or CAR, as used herein, refers to fragments, in particular amino acid chains, of an antibody TCR, BCR or CAR, that are shorter in length than the parental protein but that retain substantially the ability of the parental protein to specifically bind to an antigen, because they comprise the amino acid sequence or sequences that are responsible for the binding specificity and/or selectivity of the parental protein. As binding of a TCR to a specific antigenic peptide in a complex with MHC is defined by the CDR1 and CDR3 sequences, an antigen binding fragment of a TCR comprises at least the CDR1 and CDR3 sequences of a parental TCR. The skilled in the art is aware that the CDRs have to be interspersed with framework regions (FRs), however their specific amino acid sequences are not crucial for target antigen specificity. Further examples of functional TCR fragments include single variable domains, such as TCR alpha, beta, gamma or delta variable domains, or fragments of the α, β, δ, γ chain, such as “V_{α}-C_{α}” or “V_{β}-C_{β}” or portions thereof. Such fragments might also further comprise the corresponding hinge region. In particular aspects, the antigen-binding fragment specifically binds to complex 1A; in particular to both complex 1A and complex 2A. It has been shown that the antigen-binding function of an antibody, of a TCR, of a BCR or CAR can be performed by fragments of a full-length antibody, TCR, BCR or CAR. An antigen-binding fragment is considered to have retained substantially the binding specificity, if, for example, the binding specificity is identical to the binding specificity of the parent protein or is increased or reduced no more than 15%, 10%, 8%, 5%, 3%, 2% or 1%. In some examples, an antigen-binding fragment is considered to have retained the binding specificity, if, for example, its K_{D} to the target of the parent protein measured as outlined below is identical to the K_{D} of the parent protein or is increased or reduced no more than 10×, 5×, 3×, or 2×. The term “fragment” as used herein refers to naturally occurring fragments (e.g. splice variants or peptide fragments) as well as artificially constructed fragments, in particular to those obtained by gene-technological means. Examples of “antigen-binding fragments of an antibody” include (i) Fab fragments, monovalent fragments consisting of the VL, VH, CL and CH domains; (ii) F(ab′)₂ fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., (1989) Nature 341: 544-546), which consist of a VH domain; (vi) isolated complementarity determining regions (CDR), and (vii) combinations of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding fragment” of an antibody, BCR or CAR. The term “antigen binding portion of a TCR” comprises at least CDR1 and CDR3 of the alpha and beta chains, or gamma and delta chains, of a TCR, preferably CDR1, CDR2 and CDR3 of the alpha and beta chains, or gamma and delta chains. While these CDRs are preferably comprised in the context of their natural framework regions, they may also be comprised in another protein—a so-called protein scaffold—that positions them to each other in a similar way as they are positioned in an alpha, beta, gamma or delta chain. The antigen binding portion of a TCR comprises preferably the variable domains of the alpha and beta chains or gamma and delta chains. The antigen binding fragments of antibodies, TCRs, BCRs or CARs can be included in a monomeric, dimeric, trimeric, tetrameric or multimeric protein complex to provide such complex with one or more different antigen binding specificities. Further formats in which antigen binding fragments of an antibody are used to create monovalent, bivalent or multivalent binding molecules are known in the art and are e.g. termed diabody, tetrabody, or nanobody. Similarly to scFVs, single chain TCRs comprise the variable domains of alpha and beta chain on one protein chain linked by a linker.

The term “antigen” is used in the art to refer to a substance, preferably an immunogenic peptide that comprises at least one epitope, preferably an epitope that elicits a B or T cell response or B cell and T cell response. The term “protein antigen” refers to a protein or a portion of a protein or a protein complex that comprises an epitope that is specifically bound by the paratope of an antigen-binding protein. A protein antigen is typically a naturally occurring protein and can be of any length. It is preferred that the protein antigen comprises at least 25 amino acids. In instances where the antigenic protein is a TCR, the antigen is a complex of an antigenic peptide a MHC molecule.

The term “epitope”, also known as antigenic determinant, is the part of an antigen that is recognized by the immune system. The term epitope refers in the context of this invention to the functional epitope of an antigen. The functional epitope comprises those residues, typically amino acids or polysaccharides that usually have specific three-dimensional structural characteristics, as well as specific charge characteristics and that contribute to the non-covalent interaction between the antigen and the paratope of the antigen-binding protein. The non-covalent interaction comprises electrostatic forces, van der Walls forces, hydrogen bonds, and hydrophobic interaction. The functional epitope is a subgroup of the residues that constitute the structural epitope of an antigen binding protein. The structural epitope comprises all residues that are covered by an antigen binding protein, i.e. the footprint of an antigen binding protein. Typically, the functional epitope of an antigen bound by an antibody comprises 4 to 10 amino acids. Similarly, the functional epitope of a peptide that is MHC presented typically comprises 4 to 8 amino acids.

An “antigenic peptide” in the context of the present invention is a fragment of a protein that can bind to the peptide ligand binding pocket of a MHC molecule. Antigenic peptides can be presented by an MHC molecule on the surface of an antigen-presenting cell. Different antigenic peptides can elicit T cell responses of different intensity. In the context of the present invention, antigenic peptides eliciting a strong T cell response are described as highly immunogenic and antigenic peptides eliciting a weak T cell response are described as weakly immunogenic. Examples of antigenic peptides are viral peptides, bacterial peptides and tumor associated antigenic peptides. Antigenic peptides presented by MHC-I typically have a length of 8 to 12 amino acids. Antigenic peptides presented by MHC-II typically have a length of 13 to 25 amino acids.

A “viral antigenic peptide” in the context of the present invention is a shorter fragment of a viral protein that is presented by a major histocompatibility complex (MHC) molecule on the surface of an antigen-presenting cell, which is typically a diseased cell. The viral antigenic peptide is of a viral origin, i.e. the cell is typically infected by said virus. The viral antigenic peptide in the context of the present invention may be an antigenic peptide selected from the group consisting of human immune deficiency virus (HIV) antigenic peptides, human cytomegalovirus (HCMV) antigenic peptides, cytomegalovirus (CMV) antigenic peptides, human papillomavirus (HPV) antigenic peptides, hepatitis B virus (HBV) antigenic peptides, hepatitis C virus (HCV) antigenic peptides, Epstein-Barr virus (EBV) antigenic peptides, Influenza antigenic peptides, human adenovirus (HADV) antigenic peptides.

A “bacterial antigenic peptide” in the context of the present invention is a shorter fragment of a bacterial protein that is presented by an MHC molecule on the surface of an antigen-presenting cell, which is typically a diseased cell. The bacterial antigenic peptide is of a bacterial origin, i.e. the cell is typically infected by a bacterium. Such bacterial antigenic peptides have been discovered in the context of infections from, for example, Mycobacterium tuberculosis. Accordingly, the bacterial antigenic peptide in the context of the present invention may be a Mycobacterium tuberculosis antigenic peptide.

The term “tumor associated antigenic peptide” (TAA) refers in the context of this invention to autologous cellular antigenic peptides derived from all protein classes, such as enzymes, receptors, transcription factors, etc. that are preferentially or exclusively expressed by tumor cells. TAAs can be broadly categorized into aberrantly expressed self-antigens, mutated self-antigens, and tumor-specific antigens. TAAs that are preferentially expressed by tumor cells, are also found in normal tissues. However, their expression differs from that of normal tissues by their degree of expression in the tumor, by alterations in their protein structure in comparison with their normal counterparts, or by their aberrant subcellular localization within tumor cells. The TAAs that can be used in the methods and embodiments described herein include, for example, TAAs described in U.S. Publication 20160187351, U.S. Publication 20170165335, U.S. Publication 20170035807, U.S. Publication 20160280759, U.S. Publication 20160287687, U.S. Publication 20160346371, U.S. Publication 20160368965, U.S. Publication 20170022251, U.S. Publication 20170002055, U.S. Publication 20170029486, U.S. Publication 20170037089, U.S. Publication 20170136108, U.S. Publication 20170101473, U.S. Publication 20170096461, U.S. Publication 20170165337, U.S. Publication 20170189505, U.S. Publication 20170173132, U.S. Publication 20170296640, U.S. Publication 20170253633, U.S. Publication 20170260249, U.S. Publication 20180051080, and U.S. Publication No. 20180164315, the contents of each of these publications and sequence listings described therein, which are herein incorporated by reference in their entirety. Furthermore, the TAA in the context of the present invention is a specific ligand of MHC-class-I-molecules or MHC-class-II-molecules, preferably MHC-class-I-molecules.

The term “MHC” refers in the context of this invention to “major histocompatibility complex”. The MHC is a large locus on vertebrate DNA containing a set of closely linked polymorphic genes that code for cell surface proteins (MHC molecules) essential for the adaptive immune system. The MHC molecules have an essential role in establishing acquired immunity against altered natural or foreign proteins in vertebrates, which in turn determines histocompatibility within a tissue. The main function of MHC molecules is to bind peptides derived from self-proteins or pathogens and display them on the cell surface for recognition by appropriate T cells. The MHC gene family is divided into three subgroups: class I, class II, and class III. Complexes of peptide and MHC class I are recognized by CD8-positive T cells bearing the appropriate TCR, whereas complexes of peptide and MHC class II molecules are recognized by CD4-positive helper T cells bearing the appropriate TCR. Since both types of response, CD8 and CD4 dependent, contribute jointly and synergistically to the anti-tumor effect of T cells, the identification and characterization of tumor-associated antigens and corresponding TCRs is important in the development of cancer immunotherapies such as vaccines and cell therapies.

In the context of the present invention, the term “MHC molecule” refers to naturally occurring MHC molecules, derivatives thereof or antigenic peptide binding fragments thereof. A naturally occurring MHC-I molecule consists of an alpha chain, also referred to as MHC-I heavy chain, and a beta chain, which constitutes a β2-microglobulin molecule. The alpha chain comprises three alpha domains, i.e. α1 domain, α2 domain and α3 domain. The α1 and α2 domains mainly contribute to forming the peptide-binding groove for binding of a peptide ligand to produce a peptide MHC (pMHC) complex. MHC-I molecules typically bind peptides that are derived from cytosolic proteins and which are degraded by the proteasome after ubiquitination and subsequently transported through a specific transporter associated with antigen processing (TAP) from the cytosol to the endoplasmic reticulum (ER). MHC-I molecules typically bind peptides of 8-12 amino acids in length. A naturally occurring MHC-II molecule consists of an alpha and a beta chain, wherein the alpha chain comprises two alpha domains, α1 domain, α2 domain and the beta chain comprises two beta domains, β1 domain and β2 domain. MHC II typically fold in the ER in complex with a protein called invariant chain and are then transported to late endosomal compartments where the invariant chain is cleaved by cathepsin proteases and a short fragment remains bound to the peptide-binding groove of the MHC-II molecule, termed class II-associated invariant chain peptide (CLIP). This placeholder peptide is then normally exchanged against higher affinity peptides, which are derived from proteolytically degraded proteins available in endocytic compartments. MHC-II molecules bind peptides of 10-30 amino acids in length, typically peptides of 13-25 amino acids in length.

The term “MHC molecule derivative” refers to variants of MHC molecules that have the same peptide binding affinity and specificity as the MHC molecule from which they are derived, but may differ with respect to other characteristics, e.g. stability or binding to other molecules, e.g. CD8. In some embodiments, the MHC derivative is a stabilized MHC molecule. Preferably, a stabilized MHC molecule is a disulfide bond stabilized MHC molecule. Web 2020053398. For example, HLA-A*02:01 may be stabilized by a disulfide bridge between position 84 and 139 or 22 and 71 or 22 and 71 and 51 and 175 (wherein the positions are indicated according to IGMT nomenclature using reference sequence Acc. No. HLA0001, as indicated on www.ebi.ac.uk/ipd/imgt/hla/nomenclature/alignments.html.). In some embodiments, the MHC derivative is an MHC variant having increased or decreased binding to CD8, e.g. variant A245V (decreased binding) and variant Q115E (increased binding) described in Dockree et. al., 2017.

The term “antigenic peptide binding fragment of an MHC molecule” as used herein, refers to fragments, in particular amino acid chains, of an MHC molecule that are shorter in length than the parental, naturally occurring MHC molecule, but that retain the ability of the parental protein to specifically bind to an antigenic peptide, because they comprise the amino acid sequence or sequences that are responsible for the binding specificity and/or selectivity of the parental MHC molecule. As the antigenic peptide binds to the peptide-binding groove of an MHC molecule, an antigenic peptide binding fragment of an MHC molecule comprises at least the amino acids forming the peptide-binding groove. Antigenic peptide binding fragments of an MHC-I molecule preferably comprise the α1 and α2 domains. Antigenic peptide binding fragments of an MHC-II molecule preferably comprise the α1 and β1 domains.

The term “peptide MHC complex (pMHC complex)” refers to a complex of an MHC molecule, preferably a human MHC molecule, and an antigenic peptide. In the context of the present invention, the term (protein) complex signifies that two or more proteins (or a protein and a peptide) are linked by non-covalent protein-protein interactions. Thus, in the pMHC complex, the antigenic peptide is non-covalently bound to the MHC molecule. In particular, the antigenic peptide is located to a peptide-binding groove formed by the MHC molecule.

The term “human leukocyte antigen (HLA) complex” refers to the human MHC. In preferred embodiments of the present invention, the MHC molecule is a HLA molecule. HLA molecules differ in amino acid sequence between different human beings. However, HLAs can be identified by an internationally agreed nomenclature, the IMGT nomenclature, of HLA. The HLA-A gene is located on the short arm of chromosome 6 and encodes the larger, α-chain, constituent of HLA-A. Variation of HLA-A α-chain is key to HLA function. This variation promotes genetic diversity in the population. Since each HLA has a different affinity for peptides of certain structures, greater variety of HLAs means greater variety of antigens to be ‘presented’ on the cell surface. Each individual can express up to two types of HLA-A, one from each of their parents. Some individuals will inherit the same HLA-A from both parents, decreasing their individual HLA diversity. However, the majority of individuals receive two different copies of HLA-A. The same pattern follows for all HLA groups. In other words, every single person can only express either one or two of the 2432 known HLA-A alleles coding for currently 1740 active proteins. HLA-A*02 signifies a specific HLA allele, wherein the letter A signifies to which HLA gene the allele belongs to and the prefix*02 indicates the A2 serotype.

The term “peptide HLA complex (pHLA complex)” refers to a complex of a HLA molecule, preferably a HLA-A, HLA-B or HLA-C molecule, and an antigenic peptide.

In the context of the present invention, PB and PC are conditional peptides and PA is a rescue peptide.

The term “conditional ligand exchange” refers to a technology for generating pMHC complexes via conditional pMHC complexes. In other words, the term conditional ligand exchange refers to the exchange of a conditional peptide (PB or PC) bound to an MHC molecule with a rescue peptide (PA).

The terms “conditional peptide”, “conditional ligand” and “conditional peptide ligand” are used interchangeably. In the context of the present invention, conditional peptides are peptides that can bind to a MHC molecule and upon a defined stimulus dissociate from this MHC molecule. The defined stimulus may be a chemical and/or physical stimulus and may be selected from the group consisting of an elevation of temperature, a change of pH, contacting with periodate, contacting with dithionite, and UV radiation or a combination thereof. Conditional ligand exchange in response to an elevation of temperature is described in Luimstra et al., 2018. Conditional ligand exchange in response to a change of pH is described in WO2006/080837. Conditional ligand exchange upon contacting with periodate is described in WO2006/080837. Conditional ligand exchange upon contacting with dithionite is described in Choo et al., 2014. Conditional ligand exchange upon UV radiation is described e.g. in Toebes et al., 2006. If the defined stimulus for conditional ligand exchange is contacting with periodate, contacting with dithionite, or UV radiation, the conditional peptide usually comprises a conditionally reactive group that, when activated by the defined stimulus, effects cleavage of a covalent bond within the peptide backbone of the conditional peptide (cleavable conditionally reactive group). The term conditional peptide refers to the uncleaved form of such conditional peptides. It is envisioned that the cleaved peptide dissociates from the MHC molecule.

Suitable cleavable conditionally reactive groups are e.g. light-activatable groups (i.e. groups that can be activated by UV light, such as 3-amino-3-(-2-nitro)phenyl-propionic acid or a light-activatable structural equivalent thereof), dithionite-activatable groups (such as an azobenzene moiety), or periodate-activatable groups (such as a 1,2-dihydroxy moiety, a 1-amino-2-hydroxy moiety or 4-amino-4-deoxy-L-threonic acid).

The term “conditional pMHC complex” refers to a complex of a conditional peptide and a MHC molecule. In instances where the conditional peptide is a cleavable peptide, the conditional pMHC complex comprises the conditional peptide in its uncleaved form. This may be because (1) the conditional pMHC complex has not been exposed to the defined stimulus or because (2) the conditional pMHC complex has been exposed to the defined stimulus, but no cleavage of the conditional peptide occurred. In preferred embodiments of the present invention, the MHC molecule is a HLA molecule. Thus, in preferred embodiments, a pMHC complex is a pHLA complex and a conditional pMHC complex is a conditional pHLA complex.

The term “rescue peptide” in the context of the present invention refers to a peptide that is used to replace a conditional peptide in a pMHC complex. When the conditional peptide dissociates from the MHC molecule following exposure to the defined stimulus, the rescue peptide will bind to the empty peptide ligand binding pocket of the MHC molecule. By binding of the rescue peptide, a new (non-conditional) pMHC complex is formed. The rescue peptide preferably does not comprise a conditionally reactive group.

“UV exchange” is a preferred embodiment of a conditional ligand exchange, wherein the defined stimulus is UV radiation. A “UV radiation” stimulus refers to illumination with UV light having a wavelength of 300-400 nm, preferably 350-380 nm, more preferably about 366 nm, preferably at a temperature between 0° C. and 25° C. for about 15-90 min, preferably about 30-60 min. During the UV radiation stimulus, the pMHC/pHLA complexes are preferably in solution, more preferably in a buffer, e.g. a buffer selected from PBS and TBSA (20 nM Tris Base pH8, 150 mM NaCl, 0.02% Na-Acid).

A “UV peptide” is a preferred embodiment of a conditional peptide. The term UV peptide refers to a peptide that is cleavable (i.e., cleavage of the peptide backbone) upon UV exposure. UV peptides are generated from a parental peptide by exchanging an amino acid residue with a UV-sensitive amino acid residue (represented by the conditionally reactive group (X) in amino acid sequences). Preferably, this UV-sensitive amino acid residue is a 3-amino-3-(2-nitro)phenyl-propionic acid residue or a light-activatable structural equivalent thereof.

The term “parental peptide” refers to a peptide used as template for generation of a conditional peptide, in particular a UV peptide. Preferably, the parental peptide comprises exclusively proteinogenic amino acids. Conditional peptides are generated from parental peptides by replacing an amino acid, preferably one or two amino acids, more preferably one amino acid, with a conditionally reactive group, in particular with a conditionally reactive amino acid analogue. The conditional peptide may comprise a further amino acid substitution compared to the parental peptide. Parental peptides can be e.g. viral peptides or human peptides. In the method and embodiments of the present invention, it is preferred that the parental peptides are autologous human peptides. It is particularly preferred that the parental peptides are highly expressed and/or frequently presented in humans and can thus be expected to be non-immunogenic or only weakly immunogenic. Conditional peptides generated from non-immunogenic or weakly immunogenic parental peptides can be expected to also be non-immunogenic or only weakly immunogenic (compared to conditional peptides generated from highly immunogenic parental peptides). In preferred embodiments, the parental peptide differs from the derived conditional peptide in that one or two amino acids of the parental peptide have been replaced by a conditionally reactive amino acid analogue.

A “UV monomer” is a preferred embodiment of a conditional pHLA complex. The term “UV monomer” refers to a pHLA complex comprising a UV peptide. A UV monomer can be used to produce an EC monomer by exposing the UV monomer to UV light and providing a rescue peptide.

The term “EC monomer” refers to a pHLA complex produced from a UV monomer by UV induced peptide exchange (i.e. exchange of a UV peptide with a rescue peptide). The term EC monomer thus refers to a pHLA complex comprising PA. Multimers may be formed from several EC monomers.

The term “Std monomer” refers to a pHLA complex generated using a standard refolding protocol.

The term “K_{D}” (measured in “mol/L”, sometimes abbreviated as “M”) in the context of the present invention refers to the dissociation equilibrium constant of the particular interaction between a binding moiety (e.g. an antigen binding protein, such as a TCR) and a target molecule (e.g. an antigenic peptide in a complex with MHC). Affinity can be measured by common methods known in the art, including but not limited to surface plasmon resonance (SPR) based assay (such as the BIAcore assay); biolayer interferometry (BLI), enzyme-linked immunoabsorbent assay (ELISA); and competition assays (e.g. radio immuno assays (RIA)). Low-affinity antigen binding proteins generally bind slowly and tend to dissociate readily, whereas high-affinity antigen binding proteins generally bind faster and tend to remain bound longer. A variety of methods of measuring binding affinity are known in the art, any of which can be used for the purposes of the present invention.

“Half maximal effective concentration” also called “EC₅₀”, typically refers to the concentration of a molecule which induces a response halfway between the baseline and maximum after a specified exposure time. EC₅₀ and affinity are inversely related, the lower the EC₅₀ value the higher the affinity of the molecule. In one example, the “EC₅₀” refers to the concentration of the antigen binding protein of the invention which induces a response halfway between the baseline and maximum after a specified exposure time. EC₅₀ values can be experimentally assessed by a variety of known methods, using for example binding assays such as ELISA or flow cytometry, or functional assays.

The term “specifically binding” refers in the context of this invention to the characteristic of an antigen binding protein, such as a TCR or antibody, to specifically recognize or bind to preferably only one antigenic peptide (target antigenic peptide) and preferably show no or substantially no binding (no cross-reactivity) to another antigenic peptide (non-target antigenic peptide), i.e. to discriminate the target antigenic peptide from a non-target peptide, in particular a non-target peptide having a similar sequence as the target peptide (also referred to as similar peptide). The skilled person is aware that among the similar peptides, there will be some that are not bound by the antigen binding proteins of the invention to a detectable degree, e.g. peptides for which no binding signal or functional response beyond the background level is detectable, wherein “background level” refers to a binding signal or functional response observed for a non-homologous, “non similar” peptide, or in the absence of a peptide. For other similar peptides, a low binding, however no significant binding, may be detectable. These peptides may also be described as “potentially relevant” similar peptides.

An antigen binding protein is considered to not bind significantly to a similar peptide,
if the K_{D} for binding to the similar peptide in a complex with MHC is increased by a factor of 25 or more, 30 or more, 40 or more, 50 or more, 75 or more, or 100 or more, compared to the K_{D} for binding to the target antigenic peptide in a complex with MHC;
if in a functional assay, the response to a similar peptide in a complex with MHC is significantly reduced compared to the response to the target peptide in a complex with MHC, in particular reduced by a factor of 25 or more, 30 or more, 40 or more, 50 or more, 75 or more, or 100 or more; or
if the EC₅₀ determined in a functional assay is increased by a factor of 25 or more, 30 or more, 40 or more, 50 or more, 75 or more, or 100 or more for a similar peptide in a complex with MHC compared to the target antigenic peptide in a complex with MHC.

A “functional response” refers to a response measured in a functional assay. A “functional assay” in the context of the present invention refers to an assay in which an antigen binding protein is expressed in an immune cell and said immune cell is co-cultured with antigen presenting cells presenting an antigenic peptide in a complex with MHC. If the antigen binding protein is capable of specifically binding to the peptide:MHC complex, this leads to activation of the immune cell. Preferably, the antigen binding protein is a TCR and the immune cell is a T cell. Activation of T cells can be determined by methods known to the skilled in the art, e.g. by measuring the release of cytokines (e.g. TNF-α and/or IFN-γ) from T cells or by measuring the killing of antigen presenting cells by the T cells, e.g. by measuring the release of intercellular proteins from the antigen presenting cells. Functional assays of the latter kind are also referred to as cytotoxicity assays. Suitable intracellular proteins are e.g. endogenous LDH or a transgenic protein expressed by the antigen presenting cell, e.g. luciferase. By using different concentrations of antigenic peptide loaded on antigen presenting cells, such as T2 cells, a half maximal effective concentration (EC₅₀) can be determined in a functional assay.

The term “expression” refers in the context of this invention to the presence of a protein or peptide in human tissue. The term expression of a protein or peptide means that it is translated from its nucleic acid sequence into its amino acid sequence during the process of protein biosynthesis in the ribosomal machinery of the cell. The expressed protein can be located intracellularly or extracellularly, e.g. on the surface of cell. The human tissue wherein the protein is expressed may be healthy or diseased tissue.

“Housekeeping genes” in the context of this invention are typically constitutive genes that are required for the maintenance of basic cellular function and are expressed in all cells of an organism under normal and pathophysiological conditions. Although some housekeeping genes are expressed at relatively constant rates in most non-pathological situations, the expression of other housekeeping genes may vary depending on experimental conditions. Housekeeping genes account for majority of the active genes in the genome, and their expression is obviously vital to survival. The housekeeping gene expression levels are fine-tuned to meet the metabolic requirements in various tissues. Examples for housekeeping genes are listed (non-exhaustive) as follows: Transcription factor, translation factors, repressor molecules, RNA splicing molecules, RNA binding proteins, ribosomal proteins, mitochondrial ribosomal proteins, RNA polymerases, protein processing genes, heat shock proteins, histone, cell cycle, apoptosis, oncogenes, DNA repair, DNA replication, metabolism involved genes, e.g. genes involved in carbohydrate metabolism, citrate cycle, lipid metabolism, amino acid metabolism, NADH dehydrogenase, cytochrome C oxidase, ATPase, lysosome, proteasome, ribonuclease, thioreductases, receptors, channels, transporters, HLA/immunoglobulin/cell recognition, kinases, cytoskeletal, growth factors, tumor necrosis factor α.

The term “amino acid sequence identity” refers in the context of this invention to the percentage of sequence identity and is determined by comparing two optimally aligned sequences over a comparison window, wherein the portion of the sequence in the comparison window can comprise additions or deletions (i.e. gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” refers in the context of this invention to two or more polypeptide or nucleic acid sequences, refers to two or more sequences or subsequences that are the same, i.e. comprise the same sequence of amino acids or nucleic acids. Sequences are “substantially identical” to each other if they have a specified percentage of amino acid residues that are the same (e.g., at least 70%, at least 75%, at least 80, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity over a specified region), when compared and aligned for maximum correspondence over a comparison window, or designated region as measured using one of the following sequence comparison algorithms or by manual alignment and visual inspection. These definitions also refer to the complement of a test sequence. Accordingly, the term “at least 80% sequence identity” is used throughout the specification with regard to polypeptide and polynucleotide sequence comparisons. This expression preferably refers to a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

The term “sequence comparison” refers in the context of this invention to the process wherein one sequence acts as a reference sequence, to which test sequences are compared. When using a sequence comparison algorithm, test and reference sequences are inputted into a computer, if necessary subsequence coordinates are designated, and sequence algorithm program parameters are designated. Default program parameters are commonly used, or alternative parameters can be designated. The sequence comparison algorithm then calculates the percent sequence identities or similarities for the test sequences relative to the reference sequence, based on the program parameters. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by SEQ ID, if not specifically indicated otherwise.

Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc.

Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)). Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

Another measure of similarity provided by the BLAST algorithm is the smallest sum probability (P(N)), which provides an indication of the probability by which a match between amino acid sequences would occur by chance. For example, an amino acid is considered similar to a reference sequence if the smallest sum probability in a comparison of the test amino acid to the reference amino acid is less than about 0.2, typically less than about 0.01, and more typically less than about 0.001. Semi-conservative and especially conservative amino acid substitutions, wherein an amino acid is substituted with a chemically related amino acid are preferred. Typical substitutions are among the aliphatic amino acids, among the amino acids having aliphatic hydroxyl side chain, among the amino acids having acidic residues, among the amide derivatives, among the amino acids with basic residues, or the amino acids having aromatic residues. Typical semi-conservative and conservative substitutions are indicated in below Table 2.

TABLE 2
Amino acids and conservative and semi-
conservative substitutions, respectively.

| Amino | Conservative | Semi-conservative |
|---|---|---|
| acid | substitution | substitution |
| A | G; S; T | N; V; C |
| C | A; V; L | M; I; F; G |
| D | E; N; Q | A; S; T; K; R; H |
| E | D; Q; N | A; S; T; K; R; |
| F | W; Y; L; M; H | I; V; A |
| G | A | S; N; T; D; E; N; Q |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

Changing from A, F, H, I, L, M, P, V, W or Y to C is semi-conservative if the new cysteine remains as a free thiol. Furthermore, the skilled person will appreciate that glycines at sterically demanding positions should not be substituted and that P should not be introduced into parts of the protein which have an alpha-helical or a beta-sheet structure.

The term “detectable label” refers in the context of this invention to a molecule which labels a different molecule or a cell by allowing this different molecule to be selected due to a property or specific characteristics the label exerts. For example, the following molecules can be labelled proteins, DNA or RNA or synthetic materials such as beads or other suitable materials. Regarding proteins, labeling strategies result in the covalent attachment of different molecules, including biotin, reporter enzymes, fluorophores, magnetic labels and radioactive isotopes, to the target protein or peptide or nucleotide sequence. Single-cell can be labeled using short DNA or RNA barcode ‘tags’ to identify reads that originate from the same cell in a sequencing experiment. Labels may be a fluorescent label, e.g. xanthens, acridines, oxazines, cyanines, styryl dyes, coumarines, porphines, metal-ligand-complexes, fluorescent proteins, nanocrystals, perylenes and phtalocyanines, streptavidin-phycoerythrin (SA-PE), streptavidin-allophycocyanin (SA-APC) or streptavidin-brilliant-violet 421 (SA-BV421); RNA-barcodes or DNA-barcodes or a radioactive label. A radioactive label is typically a molecule wherein one or more atoms are replaced by the radioactive counterparts, i.e. radio isotopes. Proteins, peptides, DNA or RNA may be labeled radioactively. Magnetic labels may comprise magnetic beads or magnetic nanoparticles which can be coated with e.g. antibodies against a particular surface antigen. Magnetic labels may be used in magnetic-activated cell sorting (MACS).

The term “detectably different” refers in the context of this invention to a scenario wherein two labels are present but may only be different in the signal they are emitting. For example, two cells may be labelled with a fluorescent label, wherein the fluorescent label attached to one cell may signal in red and the fluorescent signal attached to the second cell may signal in green. The two labels of the exemplified cells are thus, detectably different.

The term “flow cytometry analysis” refers in the context of this invention to a technique comprising the measurement of chemical and physical properties of a specific cell population or cell subpopulations in a sample. The sample usually is a suspension and is adjusted to result in a flow of one cell at a time through a detection unit, typically a laser beam that excites a fluorophore and a light detector. The detected signal, e.g. light scattered by the flow through of the cell, is characteristic to the cell, i.e. its components. Multiple cells can be analyzed by this technique in a short period of time. Routine applications of flow cytometry are cell counting, cell sorting, determination of cell characteristic and functions, diagnosis of diseases, e.g. cancer, detection of biomarkers or detection of microorganisms. A popular flow cytometry technique is fluorescence activated cell sorting (FACS). The FACS technique harnesses the ability to label a target cell/cells with fluorescent dye tags or labels which allows for the cell sorting based on the individual labeling profile of a particular cell population.

The term “magnetic-activated cell sorting” (MACS) refers to a sorting technique that harnesses functional micro- or nanoparticles that are conjugated with antibodies corresponding to particular cell surface antigens. Under application of a magnetic field gradient, the magnetically targeted cells can be separated in either a positive or negative fashion with the respect to the antigen employed. A skilled person is well aware of the different kind of sorting analyses.

The term “disease” refers to an abnormal condition, especially an abnormal medical condition such as an illness or injury, wherein a tissue, an organ or an individual is not able to efficiently fulfil its function anymore. In contrast, healthy tissue, organs or individuals are referred to herein if no abnormal conditions are present and the tissue, organ or individual is without pathological finding. In healthy tissues random migration of cells is absent, cells adhere to each other in structures characterizing the tissue and assist in its function. No metastasis is present in healthy tissue. Typically, but not necessarily, a disease is associated with specific symptoms or signs indicating the presence of such disease. The presence of such symptoms or signs may thus, be indicative for a tissue, an organ or an individual suffering from a disease. An alteration of these symptoms or signs may be indicative for the progression of such a disease. A progression of a disease is typically characterized by an increase or decrease of such symptoms or signs which may indicate a deterioration or amelioration of the disease. The “deterioration” of a disease is characterized by a decreasing ability of a tissue, organ or organism to fulfil its function efficiently, whereas the “amelioration” of a disease is typically characterized by an increase in the ability of a tissue, an organ or an individual to fulfil its function efficiently. A tissue, an organ or an individual being at “risk of developing” a disease is in a healthy state but shows potential of a disease emerging. Typically, the risk of developing a disease is associated with early or weak signs or symptoms of such disease. In such case, the onset of the disease may still be prevented by treatment. Examples of a disease include but are not limited to infectious diseases, traumatic diseases, inflammatory diseases, cutaneous conditions, endocrine diseases, intestinal diseases, neurological disorders, joint diseases, genetic disorders, autoimmune diseases, and various types of cancer. Healthy tissue as defined herein usually comprises or consists of healthy cells.

The term “immune disease” refers in the context of this invention to a disease triggered by the immune system.

The term “neoplastic disease” refers in the context of this invention to diseases characterized by an abnormal growth of cells, also known as a tumor. Neoplastic diseases are conditions that cause tumor growth. Malignant tumors are cancerous and can grow slowly or quickly and carry the risk of metastasis or spreading to multiple tissues and organs. By “tumor” is meant an abnormal group of cells or tissue that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be either benign or malignant. A neoplastic disease may result in cancer, wherein exemplified cancer type diseases include but are not limited to basal cell carcinoma, bladder cancer, bone cancer, brain tumor, breast cancer, Burkitt lymphoma, cervical cancer, colon cancer, cutaneous T cell lymphoma, esophageal cancer, retinoblastoma, gastric (stomach) cancer, gastrointestinal stromal tumor, glioma, Hodgkin lymphoma, Kaposi sarcoma, leukemias, lymphomas, melanoma, oropharyngeal cancer, ovarian cancer, pancreatic cancer, pleuropulmonary blastoma, prostate cancer, throat cancer, thyroid cancer, and urethral cancer.

A “disease caused by a virus or bacteria” may also be referred to as a viral or bacterial infection. In the context of the present invention, the virus causing the disease may be selected from the group constituted of for example, human immunodeficiency viruses (HIV), Humane Cytomegalovirus (HCMV), cytomegalovirus (CMV), human papillomavirus (HPV), Hepatitis B virus (HBV), Hepatitis C virus (HCV), human papillomavirus infection (HPV), Epstein-Barr virus (EBV), Influenza virus. In the context of the present invention, the bacteria causing the disease may be Mycobacterium tuberculosis. The disease caused by this bacterium is, thus, tuberculosis. It will be understood by the skilled in the art, that when the antigen binding protein targets a viral antigenic peptide, for instance, a HIV peptide, the antigen binding protein may be for use in the treatment of HIV. Accordingly, an antigen binding protein targeting the viral or bacterial antigenic peptide is thus, suitable for use in the treatment of virus or bacteria from which said antigenic viral or bacterial antigenic peptide, is derived.

The terms “treating” or “treatment” include both therapeutic treatment (i.e. on a subject having a given disease) and/or preventive or prophylactic treatment (i.e. on a subject susceptible of developing a given disease). Therapeutic treatment and means reversing, alleviating and/or inhibiting the progress of one or more symptoms of a disorder or condition. Prophylactic treatment means preventing the occurrence of one or more symptoms of a disorder or condition. Therefore, treatment does not only refer to a treatment that leads to a complete cure of the disease, but also to treatments that slow down the progression of the disease, prevent or delay the occurrence of the disease and/or prolong the survival of the subject.

In this specification, the terms “subject”, “individual” and “patient” which are used interchangeably and refer to any mammal that may benefit from the present invention. In particular, the “individual” is a human being. In instances where the treatment is prophylactic, the individual may be healthy.

The term “subject in need thereof” in the context of this invention refers to a subject that suffers or is at risk of suffering a disease, for example an immune disease, a neoplastic disease, or a disease caused by a virus or a disease caused by bacteria. A neoplastic disease, for example cancer, involves the unregulated and/or inappropriate proliferation of cells. The neoplastic disorder or disease may be, for example, a tumor disease characterized by the expression of a tumor associated antigen (TAA).

The term “amino acid” refers in the context of this invention to the twenty natural or genetically encoded alpha-amino acids: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). The structures of these twenty natural amino acids are shown in, e.g., Stryer et al., Biochemistry, 5th ed., Freeman and Company (2002). Additional amino acids, such as selenocysteine and pyrrolysine, can also be genetically coded for (Stadtman (1996) “Selenocysteine,” Annu Rev Biochem. 65:83-100 and Ibba et al. (2002) “Genetic code: introducing pyrrolysine,” Curr Biol. 12(13):R464-R466). Amino acids can be merged into peptides, polypeptides, or proteins. As used in this specification the term “peptide” refers to a short polymer of amino acids linked by peptide bonds. It has the same chemical (peptide) bonds as proteins but is commonly shorter in length. As used in this specification, a peptide typically has a length of 8 to 25 amino acids,

The term “nucleic acid” refers in the context of this invention to single or double-stranded oligo- or polymers of deoxyribonucleotide or ribonucleotide bases or both. Nucleotide monomers are composed of a nucleobase, a five-carbon sugar (such as but not limited to ribose or 2′-deoxyribose), and one to three phosphate groups. Typically, a nucleic acid is formed through phosphodiester bonds between the individual nucleotide monomers, In the context of the present invention, the term nucleic acid includes but is not limited to ribonucleic acid (RNA) and deoxyribonucleic acid (DNA) molecules but also includes synthetic forms of nucleic acids comprising other linkages (e.g., peptide nucleic acids as described in Nielsen et al. (Science 254:1497-1500, 1991). Typically, nucleic acids are single- or double-stranded molecules and are composed of naturally occurring nucleotides. The depiction of a single strand of a nucleic acid also defines (at least partially) the sequence of the complementary strand. The nucleic acid may be single or double stranded or may contain portions of both double and single stranded sequences. Exemplified, double-stranded nucleic acid molecules can have 3′ or 5′ overhangs and as such are not required or assumed to be completely double-stranded over their entire length. The nucleic acid may be obtained by biological, biochemical or chemical synthesis methods or any of the methods known in the art, including but not limited to methods of amplification, and reverse transcription of RNA. The term nucleic acid comprises chromosomes or chromosomal segments, vectors (e.g., expression vectors), expression cassettes, naked DNA or RNA polymer, primers, probes, cDNA, genomic DNA, recombinant DNA, cRNA, mRNA, tRNA, microRNA (miRNA) or small interfering RNA (siRNA). A nucleic acid can be, e.g., single-stranded, double-stranded, or triple-stranded and is not limited to any particular length. Unless otherwise indicated, a particular nucleic acid sequence comprises or encodes complementary sequences, in addition to any sequence explicitly indicated.

The term “vector” refers in the context of this invention to a polynucleotide that encodes a protein of interest or a mixture comprising polypeptide(s) and a polynucleotide that encodes a protein of interest, which is capable of being introduced or of introducing proteins and/or nucleic acids comprised therein into a cell. Examples of vectors include but are not limited to plasmids, cosmids, phages, viruses or artificial chromosomes. A vector is used to introduce a gene product of interest, such as e.g. foreign or heterologous DNA into a host cell. Vectors may contain “replicon” polynucleotide sequences that facilitate the autonomous replication of the vector in a host cell. Foreign DNA is defined as heterologous DNA, which is DNA not naturally found in the host cell, which, for example, replicates the vector molecule, encodes a selectable or screenable marker, or encodes a transgene. Once in the host cell, the vector can replicate independently of or coincidental with the host chromosomal DNA, and several copies of the vector and its inserted DNA can be generated. In addition, the vector can also contain the necessary elements that permit transcription of the inserted DNA into an mRNA molecule or otherwise cause replication of the inserted DNA into multiple copies of RNA. Vectors may further encompass “expression control sequences” that regulate the expression of the gene of interest. Typically, expression control sequences are polypeptides or polynucleotides such as promoters, enhancers, silencers, insulators, or repressors. In a vector comprising more than one polynucleotide encoding for one or more gene products of interest, the expression may be controlled together or separately by one or more expression control sequences. More specifically, each polynucleotide comprised on the vector may be control by a separate expression control sequence or all polynucleotides comprised on the vector may be controlled by a single expression control sequence.

Polynucleotides comprised on a single vector controlled by a single expression control sequence may form an open reading frame. Some expression vectors additionally contain sequence elements adjacent to the inserted DNA that increase the half-life of the expressed mRNA and/or allow translation of the mRNA into a protein molecule. Many molecules of mRNA and polypeptide encoded by the inserted DNA can thus be rapidly synthesized. Such vectors may comprise regulatory elements, such as a promoter, enhancer, terminator and the like, to cause or direct expression of said polypeptide upon administration to a subject.

Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like. Any expression vector for animal cell can be used, as long as a gene encoding the human antibody C region can be inserted and expressed. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other examples of plasmids include replicating plasmids comprising an origin of replication, or integrative plasmids, e.g. pUC, pcDNA, pBR.

The term “T cell receptor libraries” refers in the context of the present invention to a library that contains a high number of different T cell receptor (TCR) proteins or fragments thereof, wherein each TCR protein or fragment thereof is different.

Abbreviations of frequently used terms throughout the claims and specification of the present invention:

TABLE 3
Abbreviations of frequently used terms.

| | |
|---|---|
| aAPC | Artificial antigen-presenting cell |
| ACT | adoptive cell therapy |
| APC | Antigen-presenting cell |
| APC-fluorophore | Allophycocyanin fluorophore |
| CDR | Complementarity determining regions |
| FACS | Fluorescence activated cell sorting |
| HLA | Human leukocyte antigen |
| MACS | Magnetic activated cell sorting |
| MHC-I/II | Major histocompatibility complex I/II |
| RO | Readout |
| TAA | Tumor-associated antigenic peptide |
| TCR | T cell receptor |
| TIL | Tumor infiltrating leukocyte |

### Embodiments

In the following different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

A first aspect of the invention relates to a method for selecting an immune cell expressing on its surface an antigen-binding protein specifically binding to a complex of a peptide A (PA) and a Major Histocompatibility Complex (MHC) molecule, comprising the following steps:
(i) providing a plurality of immune cells expressing different antigen-binding proteins;
(ii) contacting the plurality of immune cells with a first composition comprising:
   (a) a complex 1A, comprising a MHC molecule 1 (M1) and a peptide A (PA), and
   (b) a complex 1X, comprising M1 and a peptide B (PB);
(iii) contacting the plurality of immune cells with a second composition comprising:
   (a) a complex 2A, comprising a MHC molecule 2 (M2) and PA, and
   (b) a complex 2X, comprising M2 and a peptide C (PC);
(iv) selecting from the plurality of immune cells a cell expressing an antigen binding protein that specifically binds to complex 1A; in particular to both complex 1A and complex 2A;
wherein complexes 1X and 2X, but not 1A and 2A, dissociate upon stimulation with a defined stimulus, preferably a defined chemical and/or physical stimulus; and wherein the amino acid sequences of PB and PC differ in at least one amino acid, preferably in at least 2, 3, 4, 5, 6, 7, or 8 amino acids, more preferably at least 5, 6, 7, or 8 amino acids. A greater difference in the amino acid sequences of PB and PC is preferred over a smaller difference. The expression “PB and PC differ in at least one amino acid” is meant to specify that PB and PC differ in at least one proteinogenic amino acid. As described below, in some embodiments, PB and PC comprise a conditionally reactive group, which may be a conditionally reactive amino acid analogue. A pair of PB and PC that differs only in the conditionally reactive amino acid analogue, but not in a proteinogenic amino acid, does not fulfil the criterion that “PB and PC differ in at least one amino acid” as required by the present invention.

The first composition is obtained by (i) providing complex 1X (comprising M1 and PB), (ii) providing PA, and (iii) providing the defined stimulus, thereby effecting dissociation of PB from M1 and binding of PA to M1, resulting in formation of complex 1A, preferably wherein a composition comprising mainly complex 1A and residual amounts of complex 1X, is obtained. In preferred embodiments, the defined stimulus effects cleavage of a covalent bond in PB, thereby effecting dissociation of PB from M1.

The second composition is obtained by (i) providing complex 2X (comprising M2 and PC), (ii) providing PA, and (iii) providing the defined stimulus, thereby effecting release of PC from M2 and binding of PA to M2, resulting in formation of complex 2A, preferably wherein a composition comprising mainly complex 2A and residual amounts of complex 2X, is obtained. In preferred embodiments, the defined stimulus effects cleavage of a covalent bond in PC, thereby effecting release of PC from M2.

Preferably, the method of the first aspect of the invention comprises the steps of generating complex 1A by subjecting complex 1X to a defined stimulus and providing PA; and generating complex 2A by subjecting complex 2X to a defined stimulus and providing PA.

“Residual amounts of complex 1X or 2X” means that at least 0.1%, 1%; 2%; 3%, 4%, 5%, 6%, 7%, 8%, 9%, 10%, and/or less than 20%, 19%, 18%, 17%, 16%, 15%, 14%, 13%, 12%, 11%, 10%, 9%, 8%, 7%, 6% or 5%, of complex 1X or 2X is comprised in the first or second composition, respectively.

“Mainly complex 1A or 2A” means that at least 80%, 81%; 82%; 83%, 84%, 85%, 86%, 87%, 88%, 89%, 90%, and/or less than 99.9%, 99%, 98%, 97%, 96%, 95%, 94%, 93%, 92%, 91%, 90%, 89%, 88%, 87%, 86% or 85%, of complex 1A or 2A is comprised in the first or second composition, respectively.

In some embodiments, the immune cell is a T cell or a B cell. It is preferred that the immune cell is a T cell. More preferably, the T cell is a CD8 T cell. In some embodiments, the antigen binding protein is a TCR, a BCR or an antigen binding fragment thereof. It is most preferred that the antigen binding protein is a TCR.

The defined chemical and/or physical stimulus may be selected from the group consisting of an elevation of temperature, a change of pH, contacting with periodate, contacting with dithionite, and light, in particular UV radiation, or a combination thereof. If the defined stimulus comprises/is an elevation of temperature, it is preferred that the change in temperature is from below 10° C. to above 20° C., more preferably from below 10° C., in particular 1-10° C., to 25-45° C. If the defined stimulus comprises/is a change of pH, it is preferred that the change is from neutral pH to acidic or alkaline pH, preferably from about 7-8 to about 5-6. If the defined stimulus is contacting with dithionite, PB and/or PC comprise a dithionite-activatable group. If the defined stimulus is contacting with periodate, PB and/or PC comprise a periodate-activatable group. If the defined stimulus is UV radiation, PB and/or PC comprise a light-activatable group.

In preferred embodiments, the defined chemical and/or physical stimulus is selected from the group consisting of contacting with periodate, contacting with dithionite, and UV radiation. In some embodiments, the defined stimulus is combined with an elevation of temperature and/or a change in pH to enhance the stimulus. In most preferred embodiments, the defined stimulus is UV radiation. The skilled person is well aware of a suitable UV stimulus for peptide ligand exchange, e.g. as described in Rodenko et al., 2006, Toebes et al. 2006, Bakker et al., 2008, Chang et al, 2013 and Frosig et al., 2015.

It is preferred that M1 and/or M2, in particular M1 and M2, are MHC-I molecules. It is further preferred, that M1 and M2 are MHC-I molecules of the same allele or MHC derivatives or antigenic peptide binding fragments of the same allele. It is preferred that the MHC-I molecules are naturally occurring MHC molecules or antigenic peptide binding fragments thereof. In some examples a MHC variant having increased or decreased binding to CD8 may be used as MHC derivative.

M1 and M2 do not differ in their peptide specificity. In other words, M1 and M2 bind to the same peptide ligands. In particular, M1 and M2 bind to PA with essentially the same affinity. M1 and M2 may however differ in regions/domains that are not crucial for peptide binding, such as the alpha3 domain, if M1 and M2 are MHC-I molecules. In preferred embodiments, M1 and M2 are identical. Importantly, this does not preclude that usually M1 and M2 are bound to different carriers and/or detectable labels as will be described below.

It is preferred that M1 and/or M2 are directly or indirectly bound to a carrier. The carrier can be a cell or a synthetic carrier. The cell may be an antigen-presenting cell (APC), preferably a human APC. The synthetic carrier can be selected from a particle, a protein, in particular streptavidin, a filament, a microarray chip and an ELISA plate. The particles can be microparticles, nanoparticles, microbeads or nanobeads. Such particles are usually made of polymers. Microbeads can be magnetic or paramagnetic beads. The synthetic carrier can comprise or consist of a first member of a pair of coupling residues. In some embodiments, the synthetic carrier can be covalently or non-covalently coated with the first member of a pair of coupling residues. The second member of the pair of coupling residues is covalently or non-covalently coupled to M1 or M2. A preferred pair of first and second coupling residues comprises streptavidin and biotin. The skilled person is aware of other pairs of coupling residues. In a preferred embodiment the synthetic carrier is coated with streptavidin which will allow the immobilization of M1 or M2 comprising a biotin moiety. Conversely, a synthetic carrier coated with biotin allows the immobilization of M1 or M2 comprising a streptavidin moiety. In another preferred embodiment, the synthetic carrier consists of a first member of a pair of coupling residues that has at least two binding sites for the second member, preferably 3, 4, 5, 6, 7, or 8 binding sites and particularly preferred 4 binding sites allowing the formation of a complex with two or more of M1 (or M2), wherein each M1 (or M2) is covalently or non-covalently, preferably covalently coupled to the second member of the pair of coupling residues. In preferred aspects, streptavidin is a first member of the pair of coupling residues and biotin is a second member of the pair of coupling residues. Streptavidin has four binding sites for biotin. Thus, if complexes of a peptide and M1 (or M2) comprising biotin are contacted with streptavidin, a soluble tetramer forms in which four peptide loaded M1 (or M2) molecules are non-covalently bound to streptavidin. Such soluble multimerized pMHC complexes are particularly useful in step (iii) of the method according to the invention.

A preferred embodiment of microbeads are polystyrene beads coated with streptavidin. Microparticles, e.g. streptavidin-coated polystyrene beads, with attached peptide-MHC complexes, may also be referred to as artificial antigen-presenting cells (aAPCs). Thus, in particular the M1 and/or M2 are comprised in (an) artificial antigen-presenting cell(s) (aAPC).

In particularly preferred embodiments, M1 is bound to an APC or is bound to a microparticle, and/or M2 comprises a biotin moiety and is bound to streptavidin, in particular four M2 each comprising a biotin moiety are bound to the four subunits of the streptavidin protein, thus forming a MHC tetramer.

In preferred embodiments, M1 and/or M2, preferably M2, or the carrier that M1 and/or M2, preferably M2, are bound to, comprise a detectable label. The skilled person is well aware of how to label an MHC molecule or a carrier. The detectable labels may be the same or different. The detectable labels may be selected from the group consisting of a fluorescent label, preferably a fluorescent label selected from the group consisting of xanthens, acridines, oxazines, cynines, styryl dyes, coumarines, porphines, metal-ligand-complexes, fluorescent proteins, nanocrystals, perylenes and phtalocyanines, more preferably streptavidin-phycoerythrin (SA-PE), streptavidin-phycoerythrin-Cyanine5 (SA-PE-Cy5), streptavidin-allophycocyanin (SA-APC), streptavidin-allophycocyanin-Cyanine7 (SA-APC-Cy7), streptavidin-peridinin chlorophyll-A protein (SA-PerCP), streptavidin-peridinin chlorophyll-A protein-Cyanine5.5 (SA-PerCP/Cy5.5), streptavidin-phycoerythrin-Cyanine7 (SA-PE-Cy7), streptavidin-brilliant-violet 421 (SA-BV421), streptavidin-brilliant-violet 510 (SA-BV510), streptavidin-brilliant-violet 570 (SA-BV570), streptavidin-brilliant-violet 605 (SA-BV605), streptavidin-brilliant-violet 650 (SA-BV650), streptavidin-brilliant-violet 711, streptavidin-brilliant-violet 785SA-BV785, streptavidin VioBlue (SAVioBlue), streptavidin allophycocyanin Vio770 (SA APC Vio770), streptavidin peridinin chlorophyll-A protein Vio700 (SA PerCPVio700), streptavidin phycoerythrin Vio 770 (SA PE Vio770) or streptavidin-phycoerythrin-Dazzle594; a magnetic label; an RNA-barcode; a DNA barcode; and a radioactive label. Magnetic labels comprise magnetic beads or magnetic nanoparticles which can be coated with antibodies against a particular surface antigen. In preferred embodiments, the label is detectable by flow cytometry analysis, preferably fluorescence activated cell sorting (FACS) or microfluidic analysis or preparative sorting analysis like magnetic activated cell sorting (MACS).

In preferred embodiments, the plurality of immune cells provided in step (i) is obtained from peripheral blood of healthy subjects or subjects that suffer from a disease, or is obtained from a fraction of the peripheral blood. It is preferred that the disease is selected from the group consisting of an immune disease, a neoplastic disease, preferably cancer and/or tumora disease caused by a virus or a disease caused by bacteria. Preferably, a disease caused by a virus is a viral infection; and a disease caused by bacteria is a bacterial infection. Preferably, the viral infection is caused by a virus selected from the group consisting of HIV, HCMV, CMV, HPV, HBV, HCV, HPV, EBV, Influenza virus. More preferably, the viral infection is caused by HIV. Preferably, the bacterial infection is caused by Mycobacterium tuberculosis. Such a disease is tuberculosis. In more preferred embodiments, the disease is cancer and/or a tumor. In most preferred embodiments, the disease is cancer, such as a TAA-presenting cancer.

Preferably, the fraction is enriched in immune cells, preferably T cells, more preferably CD8 T cells or CD4 T cells. It can be envisioned that the immune cell enriched fraction is selected by detectably labeling one or more immune cell specific surface markers, preferably selected from the group consisting of CD3, CD8, CD4 and CD19. In preferred embodiments, the plurality of cells provided in step (i) are T cells that are phenotyped. Phenotyping of T cells preferably comprises the quantitative or qualitative determination of the presence of one or more T cell marker, preferably selected from the group consisting of CD3, CD4, CD8, CD11a, CD14, CD19, CD25, CD27, CD28, CD44, CD45RA, CD45RO, CD57, CD62L, CD69, CD122, CD127, CD197 (CCR7), IFNγ, IL-2, TNFα, IL7R and telomer length.

In preferred embodiments, step ii) further comprises contacting the plurality of cells with an antibody against a costimulatory molecule, preferably anti-CD28, anti-CD3, anti-CD137 and/or anti-CD134, more preferably anti-CD28 and/or anti-CD3; and/or step ii) further comprises contacting the plurality of cells with IL-2 and/or IL-12; and/or step ii) is carried out for at least 3 days, preferably at least 7 days, more preferably at least 10 days. A preferred embodiment of step (ii) is stimulation of immune cells, preferably stimulation of T cells. A particularly preferred embodiment of step (ii) is stimulation of T cells using aAPCs.

In preferred embodiments, step iii) further comprises contacting the plurality of cells with a viability dye and/or a labelled surface marker; preferably CD3, CD4, CD8 and/or CD69; more preferably CD3 and/or CD8.

The cell selected in step iv) may be a single cell or a population of cells. In preferred embodiments, step iv) comprises at least one of detection, characterization and isolation and optionally cultivation of a single cell or population of cells.

Preferred embodiments of step (iii) and (iv) are multimer staining and detection by flow cytometry, respectively. In the context of the present invention, multimer staining refers to staining of antigen binding proteins expressed on the surface of a cell with labelled peptide:MHC multimers, such as tetramers or dextramers. The peptide:MHC multimers comprise as monomer complex 2A (i.e. a PA:MHC complex) generated from complex 2X (i.e. a PC:MHC complex). Stained cells can be detected by flow cytometry.

Preferably, the peptide:MHC multimers are fluorescently labelled. An exemplary multimer staining assay is described in the material and methods section. Analysis of a multimer staining is at least one-dimensional (for complex 2A), but may also comprise further dimensions, e.g. two-dimensional with the same peptide:MHC multimer labelled with two different fluorphores, or staining for similar peptides using one or more dimensions.

Another possible embodiment of step (iii) and (iv) is a functional assay, for example, a TCR activation assay, such as an IFNγ-release assay.

One preferred application of the method according to the first aspect of the invention is to obtain information regarding the immunogenicity of antigenic peptides, in particular TAAs. In such embodiments, step (ii) is a stimulation step and step (iii) and (iv) together are a read-out step (comprising multimer staining and readout by flow cytometry). The inventors set up an in vitro immunological target validation platform comprising repeated stimulations of CD8+ T cells with aAPCs loaded with pHLA complexes and anti-CD28 antibody. Subsequently, the number of CD8+ T cells reactive to the pHLA complex is quantified.

Another preferred application of the method according to the first aspect of the invention is to isolate T cells specific for a certain antigenic peptide, in particular a TAA. In such embodiments, step (ii) is a stimulation step and step (iii) is an isolation step, preferably by FACS.

In the method according to the first aspect of the invention, an immune cell expressing on its surface an antigen-binding protein specifically binding to a complex of PA and a MHC molecule is selected. In the context of the present specification, PA can thus be described as the “target peptide (ligand)” or “peptide (ligand) of interest” or “rescue peptide”. In contrast, both PB and PC are a “conditional peptide (ligand)”. Together with M1 or M2, PB and PC form conditional pMHC complexes (complex 1X and complex 2X, respectively). These complexes are designed to dissociate upon exposure to the defined chemical and/or physical stimulus. Upon dissociation, PA binds to the peptide binding pockets of M1 and M2, leading to the formation of complex 1A and 2A, respectively.

It is preferred that PA, PB and/or PC, preferably PA, PB and PC are MHC-I binding peptides. MHC-I binding peptides have a length 8-12 amino acids, preferably 8-11 amino acids.

PA, PB and PC are presented by the same HLA allotype. HLA allotypes presenting PA, PB and PC can be selected from the group consisting of HLA-A, HLA-B, HLA-C, HLA-E, HLA-F, HLA-G, HLA-H, HLA-J, HLA-K, HLA-L. Preferably the HLA-A protein is selected from the group consisting of HLA-A1, HLA-A2, HLA-A3, and HLA-A11. Preferred HLA-A alleles are HLA-A*02:01; HLA-A*01:01, HLA-A*03:01 or HLA-A*24:02. Preferred HLA-B alleles are HLA-B*07:02; HLA-B*08:01, HLA-B*15:01, HLA-B*35:01 or HLA-B*44:05.

In the context of the present invention, the HLA allele that an antigenic peptide binds to is indicated following the designation of the antigenic peptide (in brackets). E.g. (A*03) indicates that an antigenic peptide binds to HLA-A*03.

PA is selected from an antigenic peptide, such as a viral antigenic peptide, a bacterial antigenic peptide and a tumor associated antigenic peptide (TAA). In most preferred embodiments, PA is a TAA. Examples for peptides that may be used as rescue peptide/peptide of interest in the method according to the invention are: HADV-014 (A*03), HIV-007 (B*07) (viral peptides) and MLA-001 (A*02) (TAA).

In some embodiments of the invention, PB and PC, but preferably not PA, dissociate from the pMHC complex upon an elevation of temperature and/or a change of pH. In preferred embodiments of the invention, PB and PC, but not PA, comprise a conditionally reactive group that, when activated by said defined chemical and/or physical stimulus, effects cleavage of a covalent bond within the peptide backbone of PB or PC, respectively. The cleavage results in a dissociation of the fragments of PB and PC from the pMHC complex. Such a conditionally reactive group can also be referred to as cleavable conditionally reactive group. In some embodiments, the cleavable conditionally reactive group is selected from the group consisting of a light-activatable group, periodate-activatable group and a dithionite-activatable group. In a preferred embodiment, the dithionite-activatable group comprises an azobenzene moiety. In a preferred embodiment, the periodate-activatable group comprises a 1,2-dihydroxy moiety or a functional equivalent thereof. There are alternative systems that are equivalent to the periodate and 1,2-dihydroxy system. Such systems are 1-amino-2-hydroxy systems and polyols, carbohydrates, sugar amino acid hybrids containing a periodate cleavable site. Dihydroxyethylene peptide isosters are described in Thaisrivongs et al., 1991, Thaisrivongs et al., 1993 and Ojima et al., 1998. The synthesis of 4-amino-4-deoxy-L-threonic acid (diol-containing amino acid building block) which is another periodate sensitive compound is described in Musich and Rapoport, 1978. It is preferred that the cleavable conditionally reactive group is a light-activatable group, in particular a UV sensitive group, preferably 3-amino-3-(-2-nitro)phenyl-propionic acid or a light-activatable structural equivalent thereof.

PB and PC may comprise one or two, preferably one cleavable conditionally reactive group. In instances where PB and PC comprise a cleavable conditionally reactive group, it is preferred that PB and PC differ in at least 1, 2, 3, or 4 amino acids on each side of the cleavable conditionally reactive group. The cleavable conditionally reactive group comprised in PB and PC may be the same or different. In most preferred embodiments of the invention, PB and PC, but not PA, comprise a UV sensitive group.

Generation of UV sensitive peptides from parental peptides for use in UV exchange technology is described in Toebes et al. 2006, Bakker et al., 2008, Chang et al, 2013 and Frosig et al., 2015.

Conditional peptides, and in particular UV sensitive peptides, that are suitable for use in the method of the first aspect of the present invention are described in the following paragraphs.

It is preferred that the binding between MHC molecule and conditional peptide is strong enough to ensure successful formation of the conditional pMHC complex by standard refolding techniques (preferably in the presence of β2 microglobulin) and to provide a stable pMHC complex in the absence of the defined stimulus. The binding between MHC molecule and conditional peptide should preferably be such that the conditional peptides (or their fragments) dissociate from the MHC molecule after/upon exposure to the defined stimulus. The binding strength between peptide and MHC molecule can be described using the absolute or relative Syfpeithi score or the NetMHCpan rank. In instances where the conditional peptides comprise a cleavable conditionally reactive group, Syfpeithi score and NetMHCpan rank can only be determined for the parental peptides, but not for the conditional peptides. It can however be expected that if the conditional peptides have similar binding strength to the MHC molecule as the parental peptides.

“Syfpeithi” is a scoring system that evaluates every amino acid within a given peptide to allow the prediction of T cell epitopes. The “Syfpeithi score” is calculated according to the following rules: The amino acids of a certain peptide are given a specific value depending on whether they are anchor, auxiliary anchor or preferred residue. Ideal anchors will be given 10 points, unusual anchors 6-8 points, auxiliary anchors 4-6 and preferred residues 1-4 points. Amino acids that are regarded as having a negative effect on the binding ability are given values between −1 and −3 (Rammensee et al., 1999). The “relative Syfpeithi score” is calculated as percentage of maximum score for a specific allele.

“NetMHCpan” is another scoring system to generate quantitative predictions of the affinity of peptide-MHC-I interactions using artificial neural networks (Nielsen et al., 2007).

It is preferred that the conditional peptide comprises a conditionally reactive group. In preferred embodiments, the conditionally reactive group is a conditionally reactive amino acid analogue that replaces an amino acid residue of the parental peptide. In such embodiments, it is preferred that the amino acid analogue is at a position that does not require a certain amino acid for MHC binding (lack of amino acid selectivity at this position). Preferably, the amino acid analogue is present at any position of the amino acid sequence of PB and/or PC except for the first or last position. Depending on the defined stimulus, it may also be required that the conditionally reactive amino acid analogue is located at a solvent exposed position, to ensure its accessibility for the stimulus.

It is further preferred if the complexes 1X and/or 2X (formed by an MHC molecule and PB and/or PC) have a high refolding yield, a high stability in the absence of UV exposure, in a low aggregation rate; and/or a low degradation rate. The refolding yield can be determined by e.g. Nanodrop or Bradford assay. The term “stability” in the context of the present invention refers to the stability of binding between peptide and MHC molecule in the pMHC complex. The stability of the pMHC complex, e.g. the stability at room temperature (approx. 20° C.) for e.g. 1 hour in a buffer, such as PBS, can be evaluated by ELISA. Aggregation rate and degradation rate can be evaluated qualitatively and/or quantitatively using various analytical techniques that are described in the art and are reviewed for example in Jones et al., 1993. In order to measure aggregation and degradation, a sample which comprises the pMHC complex may be exposed for a selected time period to a stress condition followed by quantitative and optionally qualitative analysis using an adequate analytical technique. In the context of the present invention, those methods refer in particular to the evaluation of degradation or to the evaluation of aggregate formation (for example using size exclusion chromatography (SEC)), by measuring turbidity (for example by dynamic light scattering (DLS) or light obscuration (LO)) and/or by visual inspection (for example by determining color and clarity).

Another important feature is a high dissociation rate after cleavage of the peptide backbone following exposure to the defined stimulus. The dissociation rate can be determined by measuring the amount of pMHC complex present after a UV stimulus as used for UV exchange, but in the absence of a rescue peptide (see Example 1).

The skilled person is aware that the decision whether a conditional peptide candidate is suitable for use in conditional ligand exchange, in particular for use in the method according to the invention, depends on a combination of the above criteria of binding strength between peptide and MHC molecule, refolding yield, stability in the absence of UV exposure, aggregation rate, degradation rate and dissociation rate after cleavage. Thus, it is understood that a conditional peptide for which e.g. the refolding yield is low, but the aggregation and degradation rates are also low, can be suitable for use in the method according to the invention.

A more detailed description of peptides for use in the method of the first aspect of the invention can be found below (see description of the peptides provided in the second aspect of the invention).

The inventors have surprisingly shown that after UV exposure, a substantial amount of residual conditional pMHC complexes remains (Example 1). This residual amount of conditional pMHC complexes is sufficient to stimulate the expansion of specific immune cells (T cells) (Example 2), even if the conditional peptides present in the conditional pMHC complexes are weakly immunogenic (Example 3). To prevent the selection of immune cells (T cells) specific for a conditional pMHC complex in step (ii), the inventors provide the inventive method according to the first aspect of the invention, which defines that in step (i) and (ii), different conditional peptides (i.e. different conditional pMHC complexes) are used (Example 4).

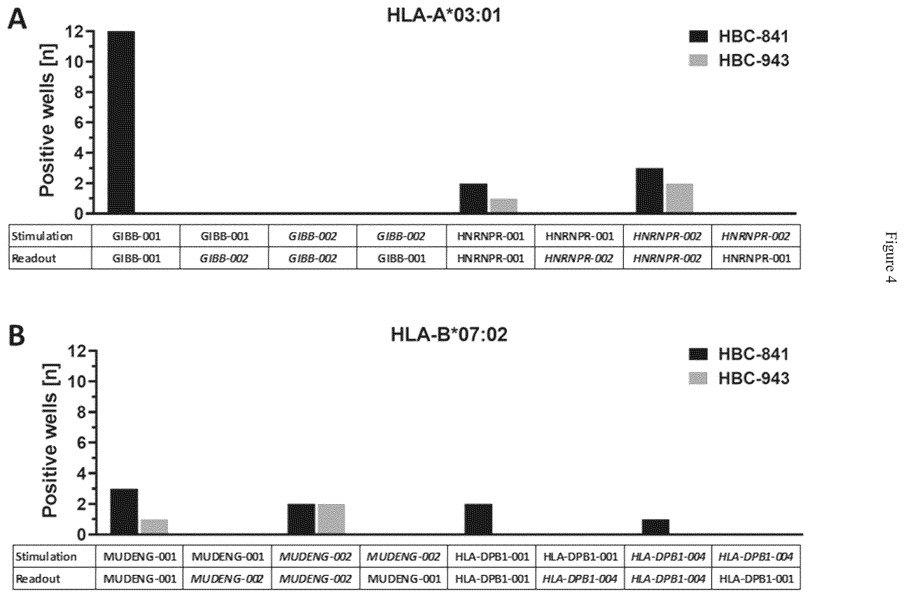

It is consequently a key feature of the invention that PB and PC differ in their amino acid sequences. In some instances, a difference of only one amino acid may be sufficient. The inventors have shown that even a difference of one amino acid is enough to prevent that T cells stimulated with a pMHC complex comprising a first peptide are detected with a pMHC complex comprising a second peptide (FIG. 4). However, it is preferred that PB and PC differ in at least 2, 3, 4, 5, 6, 7, or 8 amino acids, more preferred that PB and PC differ in at least 5, 6, 7, or 8 amino acids (higher numbers are preferred). In other words, since PB and PC preferably have a length of 8 to 12 amino acids, it is preferred that PB and PC have less than 90% amino acid identity, preferably less than 85%, 80%, 75%, 70%, 60%, 50%, 40%, 30% or 20% amino acid identity. It is also preferred that PB and PC have less than 90% amino acid similarity, preferably less than 85%, 80%, 75%, 70%, 60%, 50%, 40%, 30% or 20% amino acid similarity.

In preferred embodiments, PB and PC are selected from the group of peptides having an amino acid sequence according to SEQ ID NO: 1-66, more preferably SEQ ID NO: 1-18, 21-38, 40-56 and 58-66, more preferably SEQ ID NO: 3-18, 21-38, 40-48, 51-56, and 59-66, even more preferably SEQ ID NO: 3-14, 16-18, 21, 22, 24-26,29-38, 40-42, 44, 46-48, 51-56, and 59-66. The advantages of these peptides are described below with respect to the second aspect of the invention.

In preferred embodiments,
i) M1 and M2 is HLA-A*01:01 or a MHC derivative thereof or a antigenic peptide binding fragment thereof, and the amino acid sequences of PB and PC are selected from the group consisting of MUC16-010, MUC16-011, RNF213-010, RNF213-011, AXIN1-003, AXIN1-004, CLTC-002, CLTC-003, CLTC-004, preferably CLTC-002 and MUC16-010, more preferably PB is CLTC-002 and PC is MUC16-010, wherein MUC16-010 comprises or consists of SEQ ID NO: 1, MUC16-011 comprises or consists of SEQ ID NO: 2, RNF213-010 comprises or consists of SEQ ID NO: 3, RNF213-011 comprises or consists of SEQ ID NO: 4, AXIN1-003 comprises or consists of SEQ ID NO: 5, AXIN1-004 comprises or consists of SEQ ID NO: 6, CLTC-002 comprises or consists of SEQ ID NO: 7, CLTC-003 comprises or consists of SEQ ID NO: 8, and CLTC-004 comprises or consists of SEQ ID NO: 9; or
ii) M1 and M2 is HLA-A*02:01 or a MHC derivative thereof or a antigenic peptide binding fragment thereof, and the amino acid sequences of PB and PC are selected from the group consisting of DDX5-002, DDX5-003, DDX5-004, DDX5-005, DDX5-006, RPL19-003, RPL19-004, RPL19-005, and FLUM-003, preferably DDX5-002, DDX5-003, DDX5-004, DDX5-005, DDX5-006, RPL19-003, RPL19-004, RPL19-005, more preferably DDX5-005 and RPL19-005, even more preferably PB is DDX5-005 and PC is RPL19-005, wherein DDX5-002 comprises or consists of SEQ ID NO: 10, DDX5-003 comprises or consists of SEQ ID NO: 11, DDX5-004 comprises or consists of SEQ ID NO: 12, DDX5-005 comprises or consists of SEQ ID NO: 13, DDX5-006 comprises or consists of SEQ ID NO: 14; RPL19-002 comprises or consists of SEQ ID NO: 16, RPL19-003 comprises or consists of SEQ ID NO: 17, RPL19-002 comprises or consists of SEQ ID NO: 18; FLUM-003 comprises or consists of SEQ ID NO: 19; or
iii) M1 and M2 is HLA-A*03:01 or a MHC derivative thereof or a antigenic peptide binding fragment thereof, and the amino acid sequences of PB and PC are selected from the group consisting of UVLA3-001, GIBB-002, GIBB-003, HNRNPR-002, HNRNPR-003 and HNRNPR-004, preferably GIBB-002 and HNRNPR-002, more preferably PB is GIBB-002 and PC is HNRNPR-002, wherein UVLA3-001 comprises or consists of SEQ ID NO: 20, GIBB-002 comprises or consists of SEQ ID NO: 21, GIBB-003 comprises or consists of SEQ ID NO: 22, HNRNPR-002 comprises or consists of SEQ ID NO: 24, HNRNPR-003 comprises or consists of SEQ ID NO: 25, HNRNPR-004 comprises or consists of SEQ ID NO: 26; or
iv) M1 and M2 is HLA-A*24:02 or a MHC derivative thereof or a antigenic peptide binding fragment thereof, and the amino acid sequences of PB and PC are selected from the group consisting of TEG-002, TEG-003, TEG-004, TEG-005, TEG-006, TEG-007, PPP1CA-002, PPP1CA-003, PPP1CA-005, preferably TEG-007 and PPP1CA-005, more preferably PB is TEG-007 and PC is PPP1CA-005, wherein TEG-002 comprises or consists of SEQ ID NO: 29, TEG-003 comprises or consists of SEQ ID NO: 30, TEG-004 comprises or consists of SEQ ID NO: 31, TEG-005 comprises or consists of SEQ ID NO: 32, TEG-006 comprises or consists of SEQ ID NO: 33, TEG-007 comprises or consists of SEQ ID NO: 34, PPP1CA-002 comprises or consists of SEQ ID NO: 35, PPP1CA-003 comprises or consists of SEQ ID NO: 36, PPP1CA-005 comprises or consists of SEQ ID NO: 38; or
v) M1 and M2 is HLA-B*07:02 or a MHC derivative thereof or a antigenic peptide binding fragment thereof, and the amino acid sequences of the PB and PC are different and selected from the group consisting of UVLB7-001, HLA-DPB1-002, HLA-DPB1-003, HLA-DPB1-004, CHCHD2-003, and MUDENG-002, preferably MUDENG-002 and DPB1-004, more preferably PB is MUDENG-002 and PC is DPB1-004, wherein UVLB7-001 comprises or consists of SEQ ID NO: 39, HLA-DPB1-002 comprises or consists of SEQ ID NO: 40, HLA-DPB1-003 comprises or consists of SEQ ID NO: 41, HLA-DPB1-004 comprises or consists of SEQ ID NO: 42, CHCHD2-003 comprises or consists of SEQ ID NO: 44, and MUDENG-002 comprises or consists of SEQ ID NO: 46; or
vi) M1 and M2 is HLA-B*08:01 or a MHC derivative thereof or a antigenic peptide binding fragment thereof, and the amino acid sequences of PB and PC are selected from the group consisting of MKI-003, MKI-004, VIM-004, VIM-005, COPA-008, PDCD10-002, PDCD10-003, MIR286-002, EBV-020, SPTAN-002 and LOC646214-003, preferably MKI-003, COPA-008, PDCD10-003, MIR286-002, EBV-020, SPTAN-002 and LOC646214-003, more preferably SPTAN-002 and LOC646214-003, even more preferably PB is SPTAN-002 and PC is LOC646214-003, wherein MKI-003 comprises or consists of SEQ ID NO: 49, MKI-004 comprises or consists of SEQ ID NO: 50, VIM-004 comprises or consists of SEQ ID NO: 51, VIM-005 comprises or consists of SEQ ID NO: 52, COPA-008, comprises or consists of SEQ ID NO: 53, PDCD10-002 comprises or consists of SEQ ID NO: 54, PDCD10-003 comprises or consists of SEQ ID NO: 55, MIR286-002 comprises or consists of SEQ ID NO: 56, EBV-020 comprises or consists of SEQ ID NO: 57, SPTAN-002 comprises or consists of SEQ ID NO: 58, LOC646214-003 comprises or consists of SEQ ID NO: 59;
vii) M1 and M2 is HLA-B*44:02 or a MHC derivative thereof or a antigenic peptide binding fragment thereof, and the amino acid sequences of PB and PC are selected from the group consisting of SPTBN1-002, SPTBN1-003, SPTBN1-004, CLTC-006, CLTC-007, CSE1-005 and CSE1-006, preferably SPTBN1-004 and CSE1-005, more preferably PB is SPTBN1-004 and PC is CSE1-005, wherein SPTBN1-002 comprises or consists of the amino acid sequence SEQ ID NO: 60, SPTBN1-003 comprises or consists of SEQ ID NO: 61, SPTBN1-004 comprises or consists of SEQ ID NO: 62, CLTC-006 comprises or consists of SEQ ID NO: 63, CLTC-007 comprises or consists of SEQ ID NO: 64, CSE1-005 comprises or consists of SEQ ID NO: 65 and CSE1-006 comprises or consists of SEQ ID NO: 66; or
viii) M1 and M2 is HLA-B*44:05 or a MHC derivative thereof or a antigenic peptide binding fragment thereof, and the amino acid sequences of PB and PC are selected from the group consisting of SPTBN1-002, SPTBN1-003, SPTBN1-004, CLTC-006, CLTC-007, CSE1-005 and CSE1-006, preferably SPTBN1-004 and CLTC-006, more preferably PB is SPTBN1-004 and PC is CLTC-006, wherein SPTBN1-002 comprises or consists of the amino acid sequence SEQ ID NO: 60, SPTBN1-003 comprises or consists of SEQ ID NO: 61, SPTBN1-004 comprises or consists of SEQ ID NO: 62, CLTC-006 comprises or consists of SEQ ID NO: 63, CLTC-007 comprises or consists of SEQ ID NO: 64, CSE1-005 comprises or consists of SEQ ID NO: 65 and CSE1-006 comprises or consists of SEQ ID NO: 66.

In SEQ ID NO: 1 to SEQ ID NO: 66, the X represents a conditionally reactive group, in particular a conditionally reactive amino acid analogue comprising a light-activatable, dithionite-activatable or periodate-activatable, preferably light-activatable group, more preferably 3-amino-3-(-2-nitro)phenyl-propionic acid.

All preferred embodiments described with respect to the first aspect of the invention also apply to the following aspects of the invention, where applicable.

A second aspect of the invention relates to a conditional peptide having an amino acid sequence selected from the group consisting of SEQ ID NO: 1-19, 21-38 and 40-66, preferably SEQ ID NO: 1-14, 16-19, 21-26, 29-36, 38, 40-42, 44 and 46-66. In each sequence, the X represents a conditionally reactive group

The claimed peptides are conditional peptides comprising a conditionally reactive group, in particular a conditionally reactive amino acid analogue comprising a light-activatable, dithionite activatable or periodate activatable group. The peptides are derived from parental peptides in which one amino acid residue has been replaced with a conditionally reactive group, in particular a conditionally reactive amino acid analogue. In preferred embodiments, the conditionally reactive amino acid analogue comprises a light-activatable, dithionite-activatable or periodate-activatable group, preferably a light-activatable group. A preferred periodate-activatable group comprises a dihydroxy moiety. A preferred dithionite-activatable group comprises an azobenzene moiety. A preferred light-activatable group comprises 3-amino-3-(2-nitro)phenyl-propionic acid or a light-activatable structural equivalent thereof, e.g. 3-amino-3-(4-nitro)phenyl-propionic acid. In preferred embodiments, the peptides are UV peptides comprising a light activatable group.

The conditional peptides suitable for use in the method according to the first aspect of the invention (PB and/or PC), and the conditional peptides of the second aspect of the invention are all suitable for use in conditional ligand exchange, in particular for use in UV exchange. All peptides were tested in UV exchange reactions. Peptides that performed particularly well are identified by (++) or (+++) in Table 5.

The following features characterize both PB and/or PC (i.e. the conditional peptides of the method according to the first aspect of the invention), and the conditional peptides provided in the second aspect of the invention:
The binding strength between MHC molecule and conditional peptide is strong. In other words, the affinity between MHC molecule and conditional peptide is high. In preferred embodiments, the parental peptides of the conditional peptides have a relative Syfpeithi score of at least 0.4, preferably at least 0.5, more preferably at least 0.6, even more preferably at least 0.7 (a higher relative Syfpeithi score indicates a stronger binder); and/or have a NetMHCpan rank of 2 or less, 1.5 or less, 1 or less, 0.5 or less, preferably 0.2 or less, 0.1 or less, more preferably 0.05 or less, 0.02 or less, even more preferably 0.01 or less, or 0.005 or less (a lower NetMHCpan rank indicates a stronger binder) (Table 4). In preferred embodiments, the NetMHCpan rank refers to a NetMHCpan rank determined using NetMHCpan version 4.0.
The refolding yield (successful formation of a conditional pMHC complex by standard refolding techniques) is high. In preferred embodiments, the refolding yield is at least 0.5, at least 0.2, at least 0.1, at least 0.05, preferably at least 0.025 mol pHLA per mol HLA chain in the refolding reaction.
The conditional pMHC complexes have a high stability in the absence of the defined stimulus, in particular in the absence of UV exposure. In preferred embodiments, the conditional pMHC complexes are stable at room temperature (approx. 20° C.) in a buffer, e.g. PBS, for at least 1 hour, at least 2 hours, at least 6 hours, at least 12 hours, at least 18 hours, or at least 24 hours. The stability of the pMHC complex in the absence of the defined stimulus can be determined e.g. as pMHC monomer content as measured by SEC.
The conditional peptides have a high dissociation rate after cleavage of the peptide backbone following exposure to the defined stimulus. Preferably, at least about 15%, about 20%, about 30%, about 40%, about 50%, about 60%, about 70%, about 80%, about 85%, about 90%, or about 100% of the conditional peptide dissociates from the MHC molecule after exposure to the defined stimulus.

As described above, the inventors have surprisingly shown that after UV exposure, a substantial amount of residual conditional pMHC complexes remains (Example 1). If pMHC complexes generated by this method are used for the stimulation of T cells, this involves the risk that T cells specific for the conditional pMHC are generated. To reduce this risk, the inventors apply an additional criterion for the selection of suitable conditional peptides: the conditional peptides are selected to be non-immunogenic or only weakly immunogenic.

Conditional peptides described in the art are often derived from viral or non-human parental peptides (Toebes et al., 2006, Frosig et al. 2015, Chang et al., 2013). Such peptides have been shown to be highly immunogenic. In contrast, the peptides according to the second aspect of the invention are preferably derived from autologous human peptides. The parental peptides have been selected to be non-immunogenic or only weakly immunogenic. Thus, the conditional peptides derived thereof can also be expected to be non-immunogenic or only weakly immunogenic.

The immunogenicity of a peptide can be determined in an immunogenicity assay, e.g. by using artificial antigen presenting cells coated with peptide-MHC complexes and co-stimulatory molecules to stimulate the proliferation of human T cells. After repeated rounds of stimulation, T cells specific for the peptide-MHC will proliferate and can be detected by flow cytometry in case the peptide is immunogenic. “Weakly immunogenic peptide” in the context of the present invention means that the precursor frequency of T cells specific for that particular peptide is less than 0.1%, less than 0.01%, less than 0.001%, less than 0.001%, less than 0.0001% of T cells, in particular CD8+ T cells. Preferably, the stimulation is for 15 to 25 days, more preferably for 3 weeks, preferably in 3 subsequent stimulation cycles. Preferably, the detection is by multimer staining. Exemplary conditions for stimulation and detection are described in the materials and methods section. In preferred embodiments, stimulation of T cells, in particular CD8+ T cells, with a conditional peptide of the second aspect of the invention results in less than 10%, less than 8%, less than 6%, less than 5%, less than 3% or less than 1% of stimulated wells having detectable specific T cells for the conditional peptide, wherein each stimulated wells comprises approx. 1000 cells prior to stimulation. In preferred embodiments, under the same conditions, stimulation with a known reference peptide, in particular a viral peptide known to have a high or intermediate immunogenicity or a peptide from an immunodominant antigen such as the melanocyte/melanoma (Melan-A/MART) protein (precursor frequency at 1 in 1000), results in at least 20%, at least 30%, at least 40%, at least 50% or in some donors 100% of stimulated wells having detectable specific T cells. Preferably, the immunogenicity assay is performed using cells of at least two different donors and the numbers of detected CD8+ cells specific for the conditional peptide represent an average from at least two donors.

In addition, the immunogenicity of a peptide can be expected to be weak if a peptide is highly expressed and/or frequently presented, in particular frequently presented, in human individuals expressing the relevant HLA allotype. The “relevant HLA allotype” for the conditional peptides according to the second aspect of the invention and the parental peptides from which they are derived can be seen from Tables 4 and 5. “Frequently presented” in the context of the present invention means that a peptide has a frequency of detection of at least 10%, at least 20%, at least 30%, at least 40%, preferably at least 50%, at least 60%, more preferably at least 70%, at least 75%, at least 80%, at least 85%, at least 90%, even more preferably at least 95%, or at least 98% on normal tissue. “Normal tissue” in this context refers to a tissue which doesn't show visible micro-/macroscopic or molecular signs of (histo-)pathological alterations (e.g. due to tumors or infections) and that may be derived from any organ of a human being that expresses the relevant HLA allotype. Preferably, a frequency of detection of at least 10%, at least 20%, at least 30%, at least 40%, preferably at least 50%, at least 60%, more preferably at least 70%, at least 75%, at least 80%, at least 85%, at least 90%, even more preferably at least 95%, or at least 98% on is determined on multiple normal tissue samples derived from several different organs and/or from several different humans expressing the relevant HLA allotype. The detection frequency is determined as percentage of samples, among all tested samples of the relevant HLA allotype, where the respective peptide was detected after isolation of HLA presented peptides from normal tissues and subsequent analysis of their sequence by state-of-the-art liquid chromatography coupled mass spectrometry.

In preferred embodiments, the parental peptides have a detection frequency of at least 10%, at least 20%, at least 30%, at least 40%, at least 50%, at least 60%, at least 70%, at least 70%, at least 80%, at least 90%, at least 95%, or at least 98% on normal tissue. Such parental peptides and conditional peptides derived thereof can be expected to be only weakly immunogenic.

Peptides that are frequently presented on substantially all cells of an individual expressing the relevant HLA allotype in a tissue independent manner can be described as “housekeeping peptides”. Thus, in a preferred embodiment, the conditional peptides of the second aspect of the invention are derived from housekeeping peptides.

Preferably, the conditional peptide of the second aspect of the invention is selected from the group of peptides comprising or consisting of the amino acid sequence of SEQ ID NO: 1-18, 21-38, 40-56 or 58-66, preferably SEQ ID NO: 1-14, 16-18, 21-26, 29-36, 38, 40-42, 44, 46-56 or 58-66.

In a preferred embodiment, the conditional peptide of the second aspect of the invention is selected from the group consisting of SEQ ID NO: 1-18, 21-38, 40-56 and 58-66 preferably SEQ ID NO: 1-14, 16-18, 21-26, 29-36, 38, 40-42, 44, 46-56 and 58-66.

More preferably, the conditional peptide of the second aspect of the invention is selected from the group of peptides comprising or consisting of the amino acid sequence of SEQ ID NO: 3-18, 21-38, 40-48, 51-56, and 59-66, preferably SEQ ID NO: 3-14, 16-18, 21, 22, 24-26, 29-38, 40-42, 44, 46-48, 51-56, and 59-66.

In an even more preferred embodiment, the conditional peptide of the second aspect of the invention is selected from the group consisting of SEQ ID NO: 3-18, 21-38, 40-48, 51-56, and 59-66, preferably SEQ ID NO: 3-14, 16-18, 21, 22, 24-26, 29-38, 40-42, 44, 46-48, 51-56, and 59-66.

In some embodiments, the conditional peptide of the second aspect of the invention is specific for HLA-A*01:01, HLA-A*02:01, HLA-A*03:01, HLA-A*24:02, HLA-B*07:02, HLA-B*07:02, HLA-B*08:01 or HLA-B*44:05.

In some embodiments, the conditional peptide of the second aspect of the invention is selected from the group consisting of MUC16-010, MUC16-011, RNF213-010, RNF213-011, AXIN1-003, AXIN1-004, CLTC-002, CLTC-003, CLTC-004, preferably RNF213-010, RNF213-011, AXIN1-003, AXIN1-004, CLTC-002, CLTC-003, CLTC-004, more preferably CLTC-002, wherein MUC16-010 comprises or consists of SEQ ID NO: 1, MUC16-011 comprises or consists of SEQ ID NO: 2, RNF213-010 comprises or consists of SEQ ID NO: 3, RNF213-011 comprises or consists of SEQ ID NO: 4, AXIN1-003 comprises or consists of SEQ ID NO: 5, AXIN1-004 comprises or consists of SEQ ID NO: 6, CLTC-002 comprises or consists of SEQ ID NO: 7, CLTC-003 comprises or consists of SEQ ID NO: 8, and CLTC-004 comprises or consists of SEQ ID NO: 9. These conditional peptides are specific for HLA-A*01:01.

In some embodiments, the conditional peptide of the second aspect of the invention is selected from the group consisting of DDX5-002, DDX5-003, DDX5-004, DDX5-005, DDX5-006, RPL19-003, RPL19-004, RPL19-005, and FLUM-003, preferably DDX5-002, DDX5-003, DDX5-004, DDX5-005, DDX5-006, RPL19-003, RPL19-004 and RPL19-005, more preferably DDX5-002, DDX5-003, DDX5-004, DDX5-005, DDX5-006, RPL19-003, RPL19-004 and RPL19-005, more preferably DDX5-005 and RPL19-005, wherein DDX5-002 comprises or consists of SEQ ID NO: 10, DDX5-003 comprises or consists of SEQ ID NO: 11, DDX5-004 comprises or consists of SEQ ID NO: 12, DDX5-005 comprises or consists of SEQ ID NO: 13, DDX5-006 comprises or consists of SEQ ID NO: 14; RPL19-002 comprises or consists of SEQ ID NO: 16, RPL19-003 comprises or consists of SEQ ID NO: 17, RPL19-002 comprises or consists of SEQ ID NO: 18; FLUM-003 comprises or consists of SEQ ID NO: 19. These conditional peptides are specific for HLA-A*02:01.

In some embodiments, the conditional peptide of the second aspect of the invention is selected from the group consisting of GIBB-002, GIBB-003, HNRNPR-002, HNRNPR-003 and HNRNPR-004, preferably GIBB-002 and HNRNPR-002, wherein GIBB-002 comprises or consists of SEQ ID NO: 21, GIBB-003 comprises or consists of SEQ ID NO: 22, HNRNPR-002 comprises or consists of SEQ ID NO: 24, HNRNPR-003 comprises or consists of SEQ ID NO: 25, HNRNPR-004 comprises or consists of SEQ ID NO: 26. These conditional peptides are specific for HLA-A*03:01.

In some embodiments, the conditional peptide of the second aspect of the invention is selected from the group consisting of TEG-002, TEG-003, TEG-004, TEG-005, TEG-006, TEG-007, PPP1CA-002, PPP1CA-003, PPP1CA-005, preferably TEG-007 and PPP1CA-005, wherein TEG-002 comprises or consists of SEQ ID NO: 29, TEG-003 comprises or consists of SEQ ID NO: 30, TEG-004 comprises or consists of SEQ ID NO: 31, TEG-005 comprises or consists of SEQ ID NO: 32, TEG-006 comprises or consists of SEQ ID NO: 33, TEG-007 comprises or consists of SEQ ID NO: 34, PPP1CA-002 comprises or consists of SEQ ID NO: 35, PPP1CA-003 comprises or consists of SEQ ID NO: 36, PPP1CA-005 comprises or consists of SEQ ID NO: 38. These conditional peptides are specific for HLA-A*24:02.

In some embodiments, the conditional peptide of the second aspect of the invention is selected from the group consisting of HLA-DPB1-002, HLA-DPB1-003, HLA-DPB1-004, CHCHD2-003, and MUDENG-002, preferably MUDENG-002 and DPB1-004, wherein HLA-DPB1-002 comprises or consists of SEQ ID NO: 40, HLA-DPB1-003 comprises or consists of SEQ ID NO: 41, HLA-DPB1-004 comprises or consists of SEQ ID NO: 42, CHCHD2-003 comprises or consists of SEQ ID NO: 44, and MUDENG-002 comprises or consists of SEQ ID NO: 46. These conditional peptides are specific for HLA-B*07:02.

In some embodiments, the conditional peptide of the second aspect of the invention is selected from the group consisting of MKI-003, MKI-004, VIM-004, VIM-005, COPA-008, PDCD10-002, PDCD10-003, MIR286-002, EBV-020, SPTAN-002 and LOC646214-003, preferably MKI-003, MKI-004, VIM-004, VIM-005, COPA-008, PDCD10-002, PDCD10-003, MIR286-002, SPTAN-002 and LOC646214-003, more preferably MKI-003, MKI-004, VIM-004, VIM-005, COPA-008, PDCD10-002, PDCD10-003, MIR286-002, and LOC646214-003, even more preferably MKI-003, COPA-008, PDCD10-003, MIR286-002, EBV-020, SPTAN-002 and LOC646214-003, even more preferably SPTAN-002 and LOC646214-003, wherein MKI-003 comprises or consists of SEQ ID NO: 49, MKI-004 comprises or consists of SEQ ID NO: 50, VIM-004 comprises or consists of SEQ ID NO: 51, VIM-005 comprises or consists of SEQ ID NO: 52, COPA-008, comprises or consists of SEQ ID NO: 53, PDCD10-002 comprises or consists of SEQ ID NO: 54, PDCD10-003 comprises or consists of SEQ ID NO: 55, MIR286-002 comprises or consists of SEQ ID NO: 56, EBV-020 comprises or consists of SEQ ID NO: 57, SPTAN-002 comprises or consists of SEQ ID NO: 58, LOC646214-003 comprises or consists of SEQ ID NO: 59. These conditional peptides are specific for HLA-B*08:01.

In some embodiments, the conditional peptide of the second aspect of the invention is selected from the group consisting of SPTBN1-002, SPTBN1-003, SPTBN1-004, CLTC-006, CLTC-007, CSE1-005 and CSE1-006, preferably SPTBN1-004 and CSE1-005, wherein SPTBN1-002 comprises or consists of the amino acid sequence SEQ ID NO: 60, SPTBN1-003 comprises or consists of SEQ ID NO: 61, SPTBN1-004 comprises or consists of SEQ ID NO: 62, CLTC-006 comprises or consists of SEQ ID NO: 63, CLTC-007 comprises or consists of SEQ ID NO: 64, CSE1-005 comprises or consists of SEQ ID NO: 65 and CSE1-006 comprises or consists of SEQ ID NO: 66. These conditional peptides are specific for HLA-B*44:02.

In some embodiments, the conditional peptide of the second aspect of the invention is selected from the group consisting of SPTBN1-002, SPTBN1-003, SPTBN1-004, CLTC-006, CLTC-007, CSE1-005 and CSE1-006, preferably SPTBN1-004 and CSE1-005, wherein SPTBN1-002 comprises or consists of the amino acid sequence SEQ ID NO: 60, SPTBN1-003 comprises or consists of SEQ ID NO: 61, SPTBN1-004 comprises or consists of SEQ ID NO: 62, CLTC-006 comprises or consists of SEQ ID NO: 63, CLTC-007 comprises or consists of SEQ ID NO: 64, CSE1-005 comprises or consists of SEQ ID NO: 65 and CSE1-006 comprises or consists of SEQ ID NO: 66. These conditional peptides are specific for HLA-B*44:05.

A third aspect of the invention relates to an immune cell selected by the method of the first aspect of the invention.

A fourth aspect of the invention relates to a method for determining the sequence of a nucleic acid encoding an antigen-binding protein specifically binding to a pMHC complex 1A comprising the steps of:
(i) isolating a nucleic acid encoding the antigen-binding protein from the immune cell selected in the method of the first aspect of the invention; and
(ii) determining the sequence of the nucleic acid.

In preferred embodiments, the method of the fourth aspect comprises selecting an immune cell expressing on its surface an antigen-binding protein according to the method of the first aspect of the invention, prior to isolating from the selected immune cell a nucleic acid encoding the antigen-binding protein.

The antigen binding protein expressed on the surface of the immune cell selected by the first method of the invention is preferably a TCR. Consequently, the antigen binding protein encoded by the nucleic acid sequence determined by the method of the fourth aspect of the invention is also preferably TCR.

The following aspects of the invention refer to further methods and uses for the antigen binding protein, in particular the TCR, identified in the methods of the first and third aspect of the invention. With respect to these aspects, the term “antigen binding protein” also includes derivatives or antigen binding fragments of the antigen binding protein, in particular the TCR, identified in the methods of the first and third aspect of the invention. Thus, in one embodiment, the antigen-binding protein is a TCR or an antigen binding fragment thereof; a BCR or an antigen binding fragment thereof or an antibody or an antigen binding fragment thereof. In another embodiment, the antigen binding protein is a TCR or a part thereof comprising at least the variable domains of the alpha and beta chain. In another embodiment, the antigen binding protein comprises the CDR1 and CDR3 and optionally the CDR2 sequences of the antigen binding protein, in particular the TCR, identified in the methods of the first and fourth aspect of the invention, preferably inserted into the framework or another TCR or antibody.

A fifth aspect of the invention relates to a method for producing a host cell expressing an antigen-binding protein comprising the steps of.
(i) providing a host cell;
(ii) isolating a nucleic acid encoding the antigen-binding protein from the immune cell selected in the method of the first aspect of the invention;
(iii) producing a genetic construct comprising the nucleic acid isolated in step (ii) or a part of said nucleic acid encoding the antigen-binding protein or an antigen-binding fragment thereof; and
(iv) introducing the genetic construct into the host cell.

In preferred embodiments, the method of the fifth aspect comprises selecting an immune cell expressing on its surface an antigen-binding protein according to the method of the first aspect of the invention, prior to isolating from the selected immune cell a nucleic acid encoding the antigen-binding protein.

In one embodiment, the genetic construct is an expression vector. Preferably, the sequence encoding the antigen-binding protein or an antigen-binding fragment thereof is inserted into a suitable vector.

In preferred embodiments, the host cell is a lymphocyte, preferably a T lymphocyte or T lymphocyte progenitor cell, for example a CD4 or CD8 positive T cell; or a cell for recombinant expression, such as a Chinese Hamster Ovary (CHO) cell or a yeast cell. In one embodiment, such recombinant host cells can be used for the production of at least one antigen binding protein of the invention or part thereof. Preferably, the host cell is transformed, transduced or transfected with a nucleic acid and/or a vector encoding the antigen binding protein or antigen binding part thereof. Transduction or transfection of host cells with nucleic acid encoding the antigen binding protein or part of the antigen binding protein is conducted using methods well known in the art, for example methods described in US20190216852. In another embodiment the host cells comprising the antigen binding protein or antigen binding part thereof can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent T cell or a suspended cell, i.e., a cell that grows in suspension. For purposes of producing an antigen binding protein or part of the antigen binding protein, such as a recombinant TCR or fragment thereof, the host cell is preferably a mammalian cell. Most preferably, the host cell is a human cell. While the host cell can be of any cell type, can originate from any type of tissue, and can be of any developmental stage, the host cell preferably is a peripheral blood leukocyte (PBL) or a peripheral blood mononuclear cell (PBMC), a T cell or a B cell. More preferably, the host cell is a T cell. The T cell can be any T cell, such as a cultured T cell, e.g., a primary T cell, or a T cell from a cultured T cell line, e.g., Jurkat, SupT1, etc., or a T cell obtained from a mammal, preferably a T cell or T cell precursor from a human patient. If obtained from a mammal, the T cell can be obtained from numerous sources, including but not limited to blood, bone marrow, lymph node, the thymus, or other tissues or fluids. Preferably, the T cell is a human T cell. More preferably, the T cell is a T cell isolated from a human. The T cell can be any type of T cell and can be of any developmental stage, including but not limited to, CD4-positive and/or CD8-positive, CD4-positive helper T cells, e.g., Th1 and Th2 cells, CD8-positive T cells (e.g., cytotoxic T cells), tumor infiltrating cells (TILs), memory T cells, naive T cells. Preferably, the T cell is a CD8-positive T cell or a CD4-positive T cell. In another embodiment the host cell may be any cell for recombinant expression. Preferably, the host cell is a Chinese hamster ovary (CHO) cell.

A sixth aspect of the invention relates to a method for producing an antigen-binding protein, comprising providing a host cell produced by the method of the fifth aspect of the invention and expressing the genetic construct introduced into said host cell.

A seventh aspect of the invention relates to an antigen binding protein produced by the method of the sixth aspect of the invention.

An eighth aspect of the invention relates to a nucleic acid encoding the antigen binding protein of the seventh aspect of the invention or a vector comprising said nucleic acid.

A ninth aspect of the invention relates to a kit as defined herein below. In particular aspects, the kit is for selecting a cell expressing on its surface an antigen-binding protein specifically binding to a complex of a peptide and a MHC molecule. Said kit comprises:
a) a complex 1X, comprising a peptide B (PB) and a MHC molecule 1 (M1); and a complex 2X, comprising a peptide C (PC) and a MHC molecule 2 (M2); or
b) a peptide (PB) and a peptide (PC) and optionally (an) MHC molecule(s) to which PB and PC bind, in particular M1 and/or M2, thereby forming a complex 1X and 2X, respectively; and
c) optionally β2-microglobulin (02M);
wherein complexes 1X and 2X, dissociate upon stimulation with a defined stimulus; and wherein the amino acid sequences of PB and PC differ in at least one amino acid, preferably in at least 2, 3, 4, 5, 6, 7, or 8 amino acids. Preferably, the kit is provided for selecting an antigen binding protein specifically binding to a complex 1A comprising a peptide A (PA) and M1 or M2.

In preferred embodiments of the kit,
i) M1 and M2 is HLA-A*01:01 or a MHC derivative thereof or a antigenic peptide binding fragment thereof, and the amino acid sequences of PB and PC are selected from the group consisting of MUC16-010, MUC16-011, RNF213-010, RNF213-011, AXIN1-003, AXIN1-004, CLTC-002, CLTC-003, CLTC-004, preferably CLTC-002 and MUC16-010, more preferably PB is CLTC-002 and PC is MUC16-010, wherein MUC16-010 comprises or consists of SEQ ID NO: 1, MUC16-011 comprises or consists of SEQ ID NO: 2, RNF213-010 comprises or consists of SEQ ID NO: 3, RNF213-011 comprises or consists of SEQ ID NO: 4, AXIN1-003 comprises or consists of SEQ ID NO: 5, AXIN1-004 comprises or consists of SEQ ID NO: 6, CLTC-002 comprises or consists of SEQ ID NO: 7, CLTC-003 comprises or consists of SEQ ID NO: 8, and CLTC-004 comprises or consists of SEQ ID NO: 9; or
ii) M1 and M2 is HLA-A*02:01 or a MHC derivative thereof or a antigenic peptide binding fragment thereof, and the amino acid sequences of PB and PC are selected from the group consisting of DDX5-002, DDX5-003, DDX5-004, DDX5-005, DDX5-006, RPL19-003, RPL19-004, RPL19-005, and FLUM-003, preferably DDX5-002, DDX5-003, DDX5-004, DDX5-005, DDX5-006, RPL19-003, RPL19-004, RPL19-005, more preferably DDX5-005 and RPL19-005, even more preferably PB is DDX5-005 and PC is RPL19-005, wherein DDX5-002 comprises or consists of SEQ ID NO: 10, DDX5-003 comprises or consists of SEQ ID NO: 11, DDX5-004 comprises or consists of SEQ ID NO: 12, DDX5-005 comprises or consists of SEQ ID NO: 13, DDX5-006 comprises or consists of SEQ ID NO: 14; RPL19-002 comprises or consists of SEQ ID NO: 16, RPL19-003 comprises or consists of SEQ ID NO: 17, RPL19-002 comprises or consists of SEQ ID NO: 18; FLUM-003 comprises or consists of SEQ ID NO: 19; or
iii) M1 and M2 is HLA-A*03:01 or a MHC derivative thereof or a antigenic peptide binding fragment thereof, and the amino acid sequences of PB and PC are selected from the group consisting of UVLA3-001, GIBB-002, GIBB-003, HNRNPR-002, HNRNPR-003 and HNRNPR-004, preferably GIBB-002 and HNRNPR-002, more preferably PB is GIBB-002 and PC is HNRNPR-002, wherein UVLA3-001 comprises or consists of SEQ ID NO: 20, GIBB-002 comprises or consists of SEQ ID NO: 21, GIBB-003 comprises or consists of SEQ ID NO: 22, HNRNPR-002 comprises or consists of SEQ ID NO: 24, HNRNPR-003 comprises or consists of SEQ ID NO: 25, HNRNPR-004 comprises or consists of SEQ ID NO: 26; or
iv) M1 and M2 is HLA-A*24:02 or a MHC derivative thereof or a antigenic peptide binding fragment thereof, and the amino acid sequences of PB and PC are selected from the group consisting of TEG-002, TEG-003, TEG-004, TEG-005, TEG-006, TEG-007, PPP1CA-002, PPP1CA-003, PPP1CA-005, preferably TEG-007 and PPP1CA-005, more preferably PB is TEG-007 and PC is PPP1CA-005, wherein TEG-002 comprises or consists of SEQ ID NO: 29, TEG-003 comprises or consists of SEQ ID NO: 30, TEG-004 comprises or consists of SEQ ID NO: 31, TEG-005 comprises or consists of SEQ ID NO: 32, TEG-006 comprises or consists of SEQ ID NO: 33, TEG-007 comprises or consists of SEQ ID NO: 34, PPP1CA-002 comprises or consists of SEQ ID NO: 35, PPP1CA-003 comprises or consists of SEQ ID NO: 36, PPP1CA-005 comprises or consists of SEQ ID NO: 38; or
v) M1 and M2 is HLA-B*07:02 or a MHC derivative thereof or a antigenic peptide binding fragment thereof, and the amino acid sequences of the PB and PC are different and selected from the group consisting of UVLB7-001, HLA-DPB1-002, HLA-DPB1-003, HLA-DPB1-004, CHCHD2-003, and MUDENG-002, preferably MUDENG-002 and DPB1-004, more preferably PB is MUDENG-002 and PC is DPB1-004, wherein UVLB7-001 comprises or consists of SEQ ID NO: 39, HLA-DPB1-002 comprises or consists of SEQ ID NO: 40, HLA-DPB1-003 comprises or consists of SEQ ID NO: 41, HLA-DPB1-004 comprises or consists of SEQ ID NO: 42, CHCHD2-003 comprises or consists of SEQ ID NO: 44, and MUDENG-002 comprises or consists of SEQ ID NO: 46; or
vi) M1 and M2 is HLA-B*08:01 or a MHC derivative thereof or a antigenic peptide binding fragment thereof, and the amino acid sequences of PB and PC are selected from the group consisting of MKI-003, MKI-004, VIM-004, VIM-005, COPA-008, PDCD10-002, PDCD10-003, MIR286-002, EBV-020, SPTAN-002 and LOC646214-003, preferably MKI-003, COPA-008, PDCD10-003, MIR286-002, EBV-020, SPTAN-002 and LOC646214-003, more preferably SPTAN-002 and LOC646214-003, even more preferably PB is SPTAN-002 and PC is LOC646214-003, wherein MKI-003 comprises or consists of SEQ ID NO: 49, MKI-004 comprises or consists of SEQ ID NO: 50, VIM-004 comprises or consists of SEQ ID NO: 51, VIM-005 comprises or consists of SEQ ID NO: 52, COPA-008, comprises or consists of SEQ ID NO: 53, PDCD10-002 comprises or consists of SEQ ID NO: 54, PDCD10-003 comprises or consists of SEQ ID NO: 55, MIR286-002 comprises or consists of SEQ ID NO: 56, EBV-020 comprises or consists of SEQ ID NO: 57, SPTAN-002 comprises or consists of SEQ ID NO: 58, LOC646214-003 comprises or consists of SEQ ID NO: 59;
vii) M1 and M2 is HLA-B*44:02 or a MHC derivative thereof or a antigenic peptide binding fragment thereof, and the amino acid sequences of PB and PC are selected from the group consisting of SPTBN1-002, SPTBN1-003, SPTBN1-004, CLTC-006, CLTC-007, CSE1-005 and CSE1-006, preferably SPTBN1-004 and CSE1-005, more preferably PB is SPTBN1-004 and PC is CSE1-005, wherein SPTBN1-002 comprises or consists of the amino acid sequence SEQ ID NO: 60, SPTBN1-003 comprises or consists of SEQ ID NO: 61, SPTBN1-004 comprises or consists of SEQ ID NO: 62, CLTC-006 comprises or consists of SEQ ID NO: 63, CLTC-007 comprises or consists of SEQ ID NO: 64, CSE1-005 comprises or consists of SEQ ID NO: 65 and CSE1-006 comprises or consists of SEQ ID NO: 66; or
viii) M1 and M2 is HLA-B*44:05 or a MHC derivative thereof or a antigenic peptide binding fragment thereof, and the amino acid sequences of PB and PC are selected from the group consisting of SPTBN1-002, SPTBN1-003, SPTBN1-004, CLTC-006, CLTC-007, CSE1-005 and CSE1-006, preferably SPTBN1-004 and CLTC-006, more preferably PB is SPTBN1-004 and PC is CLTC-006, wherein SPTBN1-002 comprises or consists of the amino acid sequence SEQ ID NO: 60, SPTBN1-003 comprises or consists of SEQ ID NO: 61, SPTBN1-004 comprises or consists of SEQ ID NO: 62, CLTC-006 comprises or consists of SEQ ID NO: 63, CLTC-007 comprises or consists of SEQ ID NO: 64, CSE1-005 comprises or consists of SEQ ID NO: 65 and CSE1-006 comprises or consists of SEQ ID NO: 66.

A tenth aspect of the invention relates to a pharmaceutical composition comprising an immune cell selected by the method of the first aspect of the invention, a host cell produced by the method of the fifth aspect of the invention, an antigen binding protein of the seventh aspect of the invention, and/or a nucleic acid or vector of the eighth aspect of the invention.

An eleventh aspect of the invention relates to an immune cell selected by the method of the first aspect of the invention, a host cell produced by the method of the fifth aspect of the invention, an antigen binding protein of the seventh aspect of the invention, and/or a nucleic acid or vector of the eighth aspect of the invention for use in medicine.

A twelfth aspect of the invention relates to an immune cell selected by the method of the first aspect of the invention, a host cell produced by the method of the fifth aspect of the invention, an antigen binding protein of the seventh aspect of the invention, and/or a nucleic acid or vector of the eighth aspect of the invention for use in a method of diagnosis, treatment or prevention of a neoplastic disease.

Another aspect of the invention relates to a method for treating a neoplastic disease comprising administration of a therapeutically effective amount of an immune cell selected by the method of the first aspect of the invention and/or produced by the method of the fifth aspect of the invention and/or an antigen binding protein of the seventh aspect of the invention or a nucleic acid of the eighth aspect of the invention to a subject in need thereof. In preferred embodiments, the method comprises adoptive cell transfer.

It is preferred that the neoplastic disease in a cancer, such as a TAA-presenting cancer and that the antigen binding protein in context of the invention specifically binds to said TAA.

Yet another aspect of the invention relates to a method for treating a neoplastic disease in a subject in need thereof comprising the steps of:
(i) providing an immune cell population of the subject comprising T cells;
(ii) contacting the immune cell population of step (i) with a first composition comprising:
   (a) a complex 1A, comprising a MHC molecule 1 (M1) and a peptide A (PA), and
   (b) a complex 1X, comprising M1 and a peptide B (PB);
(iii) contacting the immune cell population with a second composition comprising:
   (a) a complex 2A, comprising a MHC molecule 2 (M2) and PA, and
   (b) a complex 2X, comprising M2 and a peptide C (PC);
(iv) selecting from the immune cell population at least one T cell expressing a TCR that specifically binds to complex 1A; in particular to both complex 1A and complex 2A; and
(v) increasing the number of the at least one selected T cell by cultivation; and
(vi) reintroducing the cultivated T cells into the subject;
wherein complexes 1X and 2X, but not 1A and 2A, dissociate upon stimulation with a defined stimulus; and wherein the amino acid sequences of PB and PC differ in at least one amino acid.

This approach is an adoptive cell transfer approach in which cells that originate from the subject in need of treatment are selected, expanded and transferred back to the subject.

A thirteenth aspect of the invention relates to a use of a peptide according to the second aspect of the invention for the preparation of pMHC complexes.

In preferred embodiments, the pMHC complexes are conditional pMHC complexes that are used for conditional ligand exchange.

The invention will now be described in more details with reference to the following figures and examples. While the invention has been illustrated and described in detail in the foregoing description, the examples are to be considered illustrative or exemplary and not restrictive.

### Example Section

### Material and Methods

Peptide Synthesis and Refolding of pHLA Molecules

Synthetic peptides were produced by standard solid phase 9-fluorenylmethyloxycarbonyl strategy using peptide synthesizer Prelude (Gyros). Purity was assessed by reversed-phase HPLC with UV detection (Thermo). To allow for UV-cleavage, an UV-sensitive 3-amino-3-(2-nitro)phenyl-propionic acid residue (abbreviated as (X) in amino acid sequences) is used to substitute one amino acid (aa) within the aa sequence of the parental peptide for generation of conditional HLA ligands (Toebes et al., 2006). Biotinylated pHLA complexes were produced from synthetic peptides and recombinant HLA molecules as described previously (Altman et al., 1996; Garboczi et al., 1992). Presence of the correct synthetic peptide in the pHLA complexes (sequence identity of synthetic peptides) was confirmed by direct infusion high resolution MS2 (Orbitrap Velos, Thermo Fisher Scientific).

pHLA Generation by UV Mediated Ligand Exchange

UV mediated peptide exchange and pHLA concentration measuring by ELISA assay were based on the description of Rodenko et al. (Rodenko et al., 2006). Briefly, UV monomers (UV-sensitive pHLA complexes) (Final concentration (f.c.): 0.5 μM for stimulation and 2 μM for readout, respectively) and rescue peptide were mixed at a molar ratio of 1:100 and irradiated with UV light (366 nm) for 30-60 min. After centrifugation, supernatant was taken and success of the exchange reaction was tested by β₂-microglobulin (β2m) ELISA.

### β2m ELISA

96 well MAXISorp plates (NUNC) were coated over night with 2 ug/ml streptavidin in PBS at room temperature, washed 4× and blocked for 1 h at 37° C. in blocking buffer (PBS with 2% BSA). Refolded HLA-A*02:01/MLA-001 monomers served as standard. UV exchange samples were incubated for 1 h at 37° C., washed 4×, incubated with 2 ug/ml HRP conjugated anti-β2m (Origene, Rockville, MD, USA) for 1 h at 37° C., washed again and detected with TMB solution (Sigma Aldrich, Taufkirchen, Germany) that is stopped with NH₂SO₄. Absorption was measured at 450 nm.

### T Cell Isolation and Culture

To perform in vitro stimulations by artificial antigen-presenting cells (aAPCs) loaded with peptide-HLA complexes (pHLA) and anti-CD28 antibody, the inventors first isolated CD8+ T cells via positive selection using CD8 microbeads (Miltenyi Biotec, Bergisch-Gladbach, Germany) from fresh HLA-matched leukapheresis products of healthy donors obtained from the University clinics Mannheim, Germany, after informed consent.

PBMCs and isolated CD8+ lymphocytes were incubated in T cell medium (TCM) until use consisting of RPMI-Glutamax (Thermo Fisher Scientific, Waltham, USA) supplemented with 10% heat-inactivated human AB serum (CC Pro, Oberdorla, Germany), 100 U/ml Penicillin/100 μg/ml Streptomycin (Biozym, Hessisch Oldendorf, Germany), 1 mM sodium pyruvate (CC Pro, Oberdorla, Germany) and 20 μg/ml Gentamycin (Biozym, Hessisch Oldendorf, Germany). 2.5 ng/ml IL-7 (PromoCell, Heidelberg, Germany) and 10 U/ml IL-2 (Novartis Pharma, Nürnberg, Germany) were also added to the TCM at this step.

Generation of aAPCs (pHLA/Anti-CD28 Coated Beads)

The purified co-stimulatory mouse IgG2a anti human CD28 Ab 9.3 (Jung et al., 1987) was chemically biotinylated using sulfo-N-hydroxysuccinimidobiotin as recommended by the manufacturer (Perbio, Bonn, Germany). Beads used were 5.6 μm diameter streptavidin coated polystyrene particles (Bangs Laboratories, Illinois, USA).

800.000 beads/200 μl were coated in 96-well plates in the presence of up to four different biotinylated pHLA complexes (total amount 50 ng/well), washed and 600 ng biotinylated anti-CD28 were added subsequently in a volume of 200 μl/well.

### T Cell Stimulation

Stimulations were initiated in 96-well plates by co-incubating 1×10⁶ CD8+ T cells with 2×10⁵ washed aAPCs in 200 μl TCM supplemented with 5 ng/ml IL-12 (PromoCell) for 3 days at 37° C. Half of the medium was then exchanged by fresh TCM supplemented with 80 U/ml IL-2 and incubation was continued for 4 days at 37° C. This stimulation cycle was performed for a total of three times.

Within each assay, pHLA complexes containing highly immunogenic peptides and pHLA complexes containing weakly immunogenic self peptides served as positive or negative control stimulation, respectively.

Generation of Fluorescent pHLA Multimers

Fluorescently labelled pHLA multimers were generated as described previously (Rodenko et al., 2006).

### Readout

For the pHLA multimer readout using up to eight different pHLA molecules per condition, a two-dimensional combinatorial coding approach was used as previously described (Andersen et al., 2012a; Andersen et al., 2012b) with minor modifications encompassing coupling to 5 different fluorochromes.

Finally, multimeric analyses were performed by staining the cells with Live/dead near IR dye (Thermo Fisher Scientific, Waltham, USA), CD8-FITC antibody clone SK1 (BD, Heidelberg, Germany) and fluorescent pHLA multimers. For analysis, a BD LSRII SORP or MACSQuant® X Flow cytometer equipped with appropriate lasers and filters was used. Peptide-specific cells were calculated as percentage of total CD8+ cells. Evaluation of multimeric analysis was done using the FlowJo™ software (FlowJo, BD, Ashland, USA). In vitro priming of specific multimer+CD8+ lymphocytes was detected by comparing to negative control stimulations.

### Sorting

For sorting of peptide specific cells, a BD FACSAria™ III SORP Cell Sorter equipped with appropriate lasers and filters is used. Cells are sorted in TCM and cultured until further use.

Isolation of HLA Peptides from Tissue Samples

HLA peptide pools from shock-frozen tissue samples were obtained by immune precipitation from solid tissues according to a slightly modified protocol (Falk et al., 1991; Seeger et al., 1999) using the HLA-A*02 specific antibody BB7.2, the HLA-A, -B, -C specific antibody w6/32, CNBr-activated sepharose, acid treatment, and ultrafiltration. More than 1000 samples derived from different tissue specimen including adipose tissue; adrenal gland; bile duct; bladder; blood cells; blood vessels; bone marrow; brain; breast; esophagus; eye; gallbladder; head and neck; heart; large intestine; small intestine; kidney; liver; lung; lymph nodes; central nerve; peripheral nerve; ovary; pancreas; parathyroid gland; peritoneum; pituitary; placenta; pleura; prostate; skeletal muscle; skin; spinal cord; spleen; stomach; testis; thymus; thyroid; trachea; ureter and uterus were tested.

### Mass Spectrometry Analyses

The HLA peptide pools as obtained were separated according to their hydrophobicity by reversed-phase chromatography (nanoAcquity UPLC system, Waters) and the eluting peptides were analyzed in LTQ velos and fusion hybrid mass spectrometers (ThermoElectron) equipped with an ESI source. Peptide pools were loaded directly onto the analytical fused-silica micro-capillary column (75 μm i.d.×250 mm) packed with 1.7 μm C18 reversed-phase material (Waters) applying a flow rate of 400 nL per minute. Subsequently, the peptides were separated using a two-step 180 minute-binary gradient from 10% to 33% B at a flow rate of 300 nL per minute. The gradient was composed of Solvent A (0.1% formic acid in water) and solvent B (0.1% formic acid in acetonitrile). A gold coated glass capillary (PicoTip, New Objective) was used for introduction into the nanoESI source. The LTQ-Orbitrap mass spectrometers were operated in the data-dependent mode using a TOP5 strategy. In brief, a scan cycle was initiated with a full scan of high mass accuracy in the orbitrap (R=30000), which was followed by MS/MS scans also in the orbitrap (R=7500) on the 5 most abundant precursor ions with dynamic exclusion of previously selected ions. Tandem mass spectra were interpreted by SEQUEST at a fixed false discovery rate (q≤0.05) and additional manual control.

In cases where the identified peptide sequence was uncertain it was additionally validated by comparison of the generated natural peptide fragmentation pattern with the fragmentation pattern of a synthetic sequence-identical reference peptide.

### Example 1: Quantification of EC Monomer Yield and Residual UV Monomers after UV Exchange

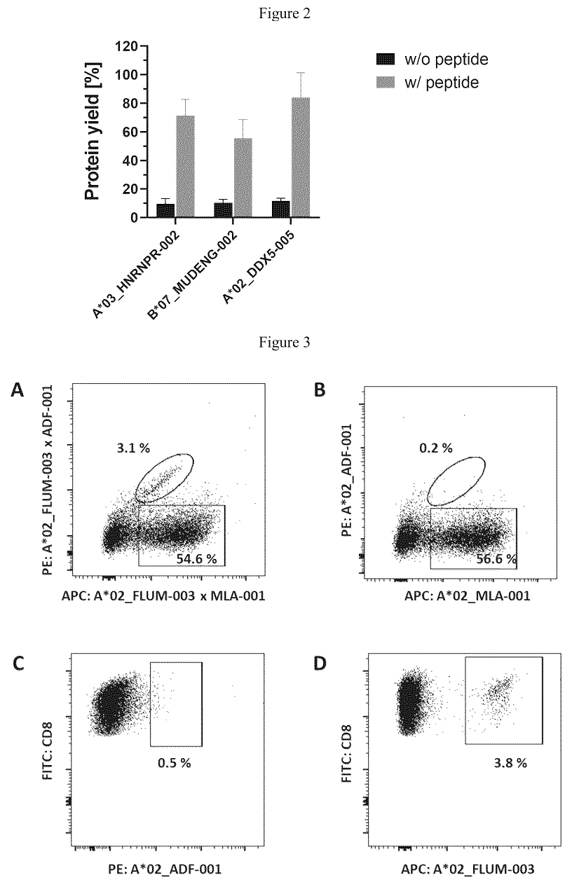

The presence of pHLA complexes after UV exposure of UV monomers was analyzed by β2m ELISA. UV exposure was performed in the presence (grey bars) or absence (black bars) of a rescue peptide. In the absence of a bound peptide, HLA molecules are instable and unfold. Surprisingly, the inventors discovered that compared to the pHLA starting concentration, 8-15% of pHLA could be detected after UV exposure in the absence of a rescue peptide, indicating that a significant number of UV monomers stay intact during UV exposure (FIG. 2). These unexpected results suggest that when EC monomers are generated via UV exchange, the UV peptides are not always exchanged completely by the rescue peptides leading to residual amounts of low concentrated UV monomers.

### Example 2: CD8+ T Cell Response Towards Residual UV Monomers

The inventors hypothesized that these background values of conditional pHLA complexes may bear the risk of erroneous results if used for stimulation or detection of T cells. The inventors were able to show that the remaining UV monomers are sufficient to activate and detect T cells recognizing UV peptides.

CD8+ T cells were stimulated with artificial antigen-presenting cells (aAPCs) coated with A*02:01_FLUM-003×MLA-001 molecules where FLUM-003 is the conditional ligand that has been exchanged by MLA-001. In addition to the strong APC single positive population, a second PE+ APC+ double positive population could be detected when cells were stained with a combination of two different multimers whose pHLA monomers derived from UV exchange using FLUM-003 as conditional ligand (APC: A*02:01_FLUM-003×MLA-001 and PE: A*02:01_FLUM-003×ADF-001) (FIG. 3, A). This population was not observed if cells from the same stimulation well were stained with multimers whose pHLA monomers were generated by standard pHLA refolding (FIG. 3, B). Multimer staining with A*02:01_FLUM-003 or A*02:01_ADF-001 specific multimers derived from standard pHLA refolding confirmed the presence of FLUM-003 but not ADF-001 specific cells (FIG. 3, C+D). These results clearly show that even low amounts of remaining conditional pHLA molecules are sufficient to detect cells specific for the conditional ligand by multimer staining.

### Example 3: Immunogenicity of Parental Peptides and UV Peptides Derived from Human Proteins

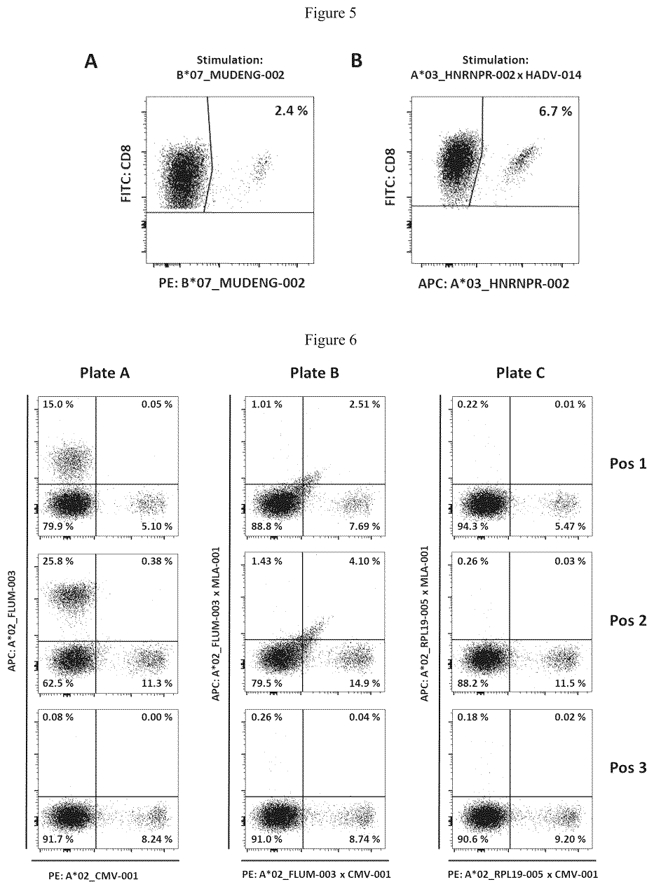

As the UV peptide FLUM-003 (KILGFVF(X)V) derives from the highly immunogenic Influenca A Matrix peptide GILGFVFTL (FLUM-001), the inventors analyzed if UV peptides derived from weakly immunogenic human proteins also bear the risk to stimulate unwanted T cell populations as there are high numbers of self-reactive CD8+ cells in the blood of healthy humans. The inventors have shown that there is stimulation and expansion of peptide specific T cell populations after in vitro priming with aAPCs loaded with pHLA molecules comprising UV peptides derived from weakly immunogenic human proteins or their respective parental peptides (FIG. 4). In a further experiment, CD8+ cells were either stimulated using standard refolded pHLA molecules carrying a human UV peptide (B*07:02_MUDENG-002 in FIG. 5, A) or pHLA molecules obtained after UV exchange using a UV monomer comprising a human UV peptide (A*03:01_HNRNPR-002×HADV-014; FIG. 5, B). In both cases, UV peptide-specific T cell populations of 2.4% or 6.7% of CD8+ cells, respectively, could be detected by multimer staining. This indicates that also UV peptides derived from human source proteins can stimulate and expand specific T cell populations, even at low concentrations as in the case of residual UV monomers present after UV exchange (FIG. 5, B).

### Example 4: Using UV Monomers with Different UV Peptides for Stimulation and Detection Prevents Detection of False Positive Signals

Based on their results, the inventors set out to investigate whether using UV monomers with identical UV peptides for the UV-mediated generation of pHLA molecules for both stimulation and detection of peptide-specific T cells could lead to false positive results as one would not be able to differentiate if the detected cells are specific for the rescue peptide or the UV peptide.

The inventors analyzed CD8+ T cells after priming with aAPCs coated with anti-CD28 mAb and HLA-A*02_FLUM-003×CMV-001. For readout cells were split and either stained with pHLA multimers derived from standard refolded pHLA molecules (FIG. 6, Plate A), pHLA molecules generated by UV-mediated ligand exchange of FLUM-003 (which has also been used for stimulation) (FIG. 6, Plate B) or pHLA molecules generated by UV-mediated ligand exchange of another UV monomer, RPL19-005 (FIG. 6, Plate C). The inventors could show that in some cases (see FIG. 6, stimulation position 1 and 2) FLUM-003 specific cells were stimulated and detected if using the same UV monomer whereas using another UV monomer for generation of pHLAs for multimer staining did prevent this unwanted detection of FLUM-003 specific cells. Staining of wells without FLUM-003 specific cells (FIG. 6, Position 3) confirmed that detection of double positive populations is not due to unspecific staining of pHLAs generated by UV-mediated ligand exchange of FLUM-003 but specific detection by low amounts of remaining A*02_FLUM-003 in the multimer solution.

With the inventive approach of using two UV monomers each comprising a different UV peptide, which was established for different HLA allotypes, detection of false positive signals can be prevented.

TABLE 4
Parental peptides

| SEQ | | | | | | | | | | |
|---|---|---|---|---|---|---|---|---|---|---|
| ID | | Peptide | Allele | | SYFPEITHI_ | netMHCpan | Normals | frequency | Human | House-keeping |
| NO | sequence | Code | Name | SYFPEITHI | rel | rank | frequency | Tumor | origin | peptide |
| 67 | TPKEKAQAL | MKI-002 | B*0801 | 39 | 0.91 | 0.005 | 0% | 4% | YES | NO |
| 68 | FLRGRAYGL | EBV-017 | B*0801 | 31 | 0.72 | 0.017 | 0% | 0% | NO | NO |
| 69 | GILGFVFTL | FLUM-001 | A*0201 | 30 | 0.83 | 0.083 | 0% | 0% | NO | NO |
| 70 | STETSTVLY | MUC16-009 | A*0101 | 35 | 0.7 | 0.005 | 0% | 15% | YES | NO |
| 71 | NIKKKQEAL | SPTAN-001 | B*0801 | 35 | 0.81 | 0.006 | 2% | 10% | YES | NO |
| 72 | TVKQKYLSF | LOC646214-002 | B*0801 | 26 | 0.6 | 0.029 | 30% | 72% | YES | YES |
| 73 | DLNEKARAL | PDCD10-001 | B*0801 | 29 | 0.67 | 0.018 | 56% | 83% | YES | YES |
| 74 | IIYKGGTSR | GSN-001 | A*0301 | 26 | 0.59 | 0.05 | 69% | 74% | YES | YES |
| 75 | FGLARAFSL | MIR286-001 | B*0801 | 19 | 0.44 | 0.195 | 70% | 92% | YES | YES |
| 76 | SPRPPLISV | MUDENG-001 | B*0702 | 22 | 0.67 | 0.004 | 72% | 93% | YES | YES |
| 77 | ILMEHIHKL | RPL19-001 | A*0201 | 32 | 0.89 | 0.003 | 75% | 86% | YES | YES |
| 78 | DLERKVESL | VIM-003 | B*0801 | 27 | 0.63 | 0.145 | 89% | 99% | YES | YES |
| 79 | AEIDARNDSF | SPTBN1-001 | B*4402 | 27 | 0.75 | 0.016 | 90% | 89% | YES | YES |
| 80 | APRTVALTAL | HLA-DPB1-001 | B*0702 | 26 | 0.87 | 0.008 | 92% | 98% | YES | YES |
| 81 | NENSLFKSL | CLTC-005 | B*4402 | 24 | 0.65 | 0.068 | 93% | 98% | YES | YES |
| 82 | ALFLTLTTK | GIBB-001 | A*0301 | 29 | 0.66 | 0.028 | 93% | 99% | YES | YES |
| 83 | STDPRPASY | AXIN1-002 | A*0101 | 37 | 0.74 | 0.003 | 93% | 97% | YES | YES |
| 84 | VTELHVISY | RNF213-009 | A*0101 | 37 | 0.74 | 0.019 | 95% | 96% | YES | YES |
| 85 | DAVVKHVL | COPA-007 | B*0801 | 25 | 0.63 | 0.101 | 95% | 95% | YES | YES |
| 86 | AYVHMVTHF | TEG-001 | A*2402 | 22 | 0.71 | 0.016 | 96% | 99% | YES | YES |
| 87 | AESIVVHTY | CSE1-004 | B*4402 | 27 | 0.73 | 0.001 | 96% | 97% | YES | YES |
| 88 | LLDQGQLNKY | CLTC-001 | A*0101 | 28 | 0.58 | 0.033 | 96% | 98% | YES | YES |
| 89 | RLFVGSIPK | HNRNPR-001 | A*0301 | 26 | 0.59 | 0.012 | 96% | 98% | YES | YES |
| 90 | APRQPGLMA | CHCHD2-001 | B*0702 | 26 | 0.79 | 0.039 | 98% | 99% | YES | YES |
| 91 | YLLPAIVHI | DDX5-001 | A*0201 | 30 | 0.83 | 0.006 | 98% | 99% | YES | YES |
| 92 | KYPENFFLL | PPP1CA-001 | A*2402 | 29 | 0.94 | 0.005 | 100% | 100% | YES | YES |

TABLE 5
Candidate conditional peptides

| | | | | | Suitable for |
|---|---|---|---|---|---|
| | | conditional | | | conditional |
| HLA | Parental peptide | peptide candidate | | SEQ | peptide |
| restriction | Peptide code | Peptide code | Sequence | ID NO | exchange |
| A*01:01 | MUC16-009 | MUC16-010 | STETS(X)VLY | 1 | +++ (R) |
| | | MUC16-011 | STETST(X)LY | 2 | ++ |
| | RNF213-009 | RNF213-010 | VTELH(X)ISY | 3 | ++ |
| | | RNF213-011 | VTELHV(X)SY | 4 | ++ |
| | AXIN1-002 | AXIN1-003 | STDPR(X)ASY | 5 | ++ |
| | | AXIN1-004 | STDPRP(X)SY | 6 | ++ |
| | CLTC-001 | CLTC-002 | LLDQG(X)LNKY | 7 | +++ (S) |
| | | CLTC-003 | LLDQGQ(X)NKY | 8 | ++ |
| | | CLTC-004 | LLDQGQL(X)KY | 9 | ++ |
| A*02:01 | DDX5-001 | DDX5-002 | YLLPA(X)VHI | 10 | ++ |
| | | DDX5-003 | YL(X)PAIVHI | 11 | + |
| | | DDX5-004 | YA(X)PAIVHI | 12 | ++ |
| | | DDX5-005 | YM(X)PAIVHT | 13 | +++ (S) |
| | | DDX5-006 | YA(X)PA(X)VHI | 14 | ++ |
| | RPL19-001 | RPL19-002 | IL(X)EHIHKL | 15 | − |
| | | RPL19-003 | ILMEH(X)HKL | 16 | ++ |
| | | RPL19-004 | IAMEH(X)HKL | 17 | ++ |
| | | RPL19-005 | IL(X)EIHKL | 18 | +++ (R) |
| | FLUM-001 | FLUM-003 | KILGFVF(X)V | 19 | ++ |
| A*03:01 | unknown | UVLA3-001 | RIYR(X)GATR | 20 | ++ |
| | GIBB-001 | GIBB-002 | AI(X)LTLTTK | 21 | +++ (S) |
| | | GIBB-003 | AL(X)LTLTTR | 22 | ++ |
| | | GIBB-004 | AI(X)LTLTTR | 23 | − |
| | HNRNPR-001 | HNRNPR-002 | RL(X)VGSIPR | 24 | +++ (R) |
| | | HNRNPR-003 | RI(X)VGSIPK | 25 | ++ |
| | | HNRNPR-004 | RI(X)VGSIPR | 26 | ++ |
| | GSN-001 | GSN-002 | II(X)KGGTSR | 27 | − |
| | | GSN-003 | IA(X)KGGTSR | 28 | − |
| A*24:02 | TEG-001 | TEG-002 | AY(X)HMVTHF | 29 | + |
| | | TEG-003 | AYVHM(X)THF | 30 | + |
| | | TEG-004 | AYVHMV(X)HF | 31 | + |
| | | TEG-005 | AT(X)HMVTHF | 32 | + |
| | | TEG-006 | AF(X)HMVTHF | 33 | + |
| | | TEG-007 | AF(X)HMV(X)HF | 34 | +++ (S) |
| | PPP1CA-001 | PPP1CA-002 | KYP(X)NFFLL | 35 | + |
| | | PPP1CA-003 | KYP(X)NF(X)LL | 36 | + |
| | | PPP1CA-004 | KYPEN(X)FLL | 37 | − |
| | | PPP1CA-005 | KFP(X)NF(X)LL | 38 | +++ (R) |
| B*07:02 | unknown | UVLB7-001 | AARG(X)TLAM | 39 | ++ |
| | HLA-DPB1-001 | HLA-DPB1-002 | AART(X)ALTAL | 40 | ++ |
| | | HLA-DPB1-003 | AART(X)ALTAM | 41 | ++ |
| | | HLA-DPB1-004 | AARTVALT(X)M | 42 | +++ (R) |
| | CHCHD2-001 | CHCHD2-002 | AARQPG(X)MA | 43 | − |
| | | CHCHD2-003 | APRQPG(X)MA | 44 | ++ |
| | | CHCHD2-004 | AARQPGL(X)A | 45 | − |
| | MUDENG-001 | MUDENG-002 | SARPPL(X)SV | 46 | +++ (S) |
| | | MUDENG-003 | SARPP(X)ISV | 47 | ++ |
| | | MUDENG-004 | SARPPL(X)SM | 48 | ++ |
| B*08:01 | MKI-002 | MKI-003 | TPKEKA(X)AL | 49 | ++ |
| | | MKI-004 | TPKEKAQ(X)L | 50 | + |
| | VIM-003 | VIM-004 | DLERKV(X)SL | 51 | + |
| | | VIM-005 | DLERKVE(X)L | 52 | + |
| | COPA-007 | COPA-008 | DAVVKH(X)L | 53 | ++ |
| | PDCD10-001 | PDCD10-002 | DLNEKA(X)AL | 54 | + |
| | | PDCD10-003 | DLNEKAR(X)L | 55 | ++ |
| | MIR286-001 | MIR286-002 | FGLARA(X)SL | 56 | ++ |
| | EBV-017 | EBV-020 | FLRGRA(X)GL | 57 | ++ |
| | SPTAN-001 | SPTAN-002 | NIKKKQ(X)AL | 58 | +++ (R) |
| | LOC646214-002 | LOC646214-003 | TVKQKY(X)SF | 59 | +++ (S) |
| B*44:02/ | SPTBN1-001 | SPTBN1-002 | AEIDAR(X)DSF | 60 | ++ |
| B*44:05 | | SPTBN1-003 | AEIDARN(X)SF | 61 | ++ |
| | | SPTBN1-004 | AEIDARND(X)F | 62 | +++ (S) |
| | CLTC-005 | CLTC-006 | NENSLF(X)SL | 63 | +++ (S) |
| | | CLTC-007 | NENSLFK(X)L | 64 | ++ |
| | CSE1-004 | CSE1-005 | AESIVV(X)TY | 65 | +++ (R) |
| | | CSE1-006 | AESIVVH(X)Y | 66 | ++ |

### REFERENCES

Altman J D, Moss P A, Goulder P J, Barouch D H, McHeyzer-Williams M G, Bell J I, McMichael A J, Davis M M (1996). Phenotypic analysis of antigen-specific T lymphocytes. Science 274, 94-96.
Andersen R S, Kvistborg P, Frosig T M, Pedersen N W, Lyngaa R, Bakker A H, Shu C J, Straten P, Schumacher T N, Hadrup S R (2012b). Parallel detection of antigen-specific T cell responses by combinatorial encoding of MHC multimers. Nat Protoc 7, 891-902.
Bakker A H, Hoppes R, Linnemann C, Toebes M, Rodenko B, Berkers C R, Hadrup S R, van Esch W J E, Heemskerk M H M, Ovaa H, Schumacher T N M (2008). Conditional M H C class I ligands and peptide exchange technology for the human MHC gene products HLA-A1, -A3, -A11, and -B7. Proc Natl Acad Sci USA., 105(10):3825-30
Chang C X L, Tan A T, Or M Y, Toh K Y, Lim P Y, Chia A S E, Froesig T M, Nadua K D, Oh H-U, Leong H N, Hadrup S R, Gehring A J, Tan Y-J, Bertoletti A Grotenbreg G M (2013). Conditional ligands for Asian H L A variants facilitate the definition of CD8+ T-cell responses in acute and chronic viral diseases. Eur. J. Immunol. 2013. 43: 1109-1120
Choo J A L, Thong S Y, Yap J, van Esch W J E, Raida M, Meijers R, Lescar J, Verhelst S H L, Grotenbreg G M (2014). Bioorthogonal Cleavage and Exchange of Major Histocompatibility Complex Ligands by Employing Azobenzene-Containing Peptides. Angew Chem Int Ed Engl., 53(49):13390-4.
Dockree T, Holland C J, Clement M, Ladell K, McLaren J E, van den Berg H A, Gostick E, Miners K L, Llewellyn-Lacey S, Bridgeman J S, Man S, Bailey M, Burrows S R, Price D A, Wooldridge L (2017). CD8+ T-cell specificity is compromised at a defined MHCI/CD8 affinity threshold. Immunol Cell Biol. 95, 68-76
Falk K, Rötzschke O, Stevanović S, Jung G, Rammensee H G (1991). Allele-specific motifs revealed by sequencing of self-peptides eluted from MHC molecules. Nature 351: 290-296
Frosig T M, Yap J, Seremet T, Lyngaa R, Svane I M, Straten P T, Heemskerk M H M, Grotenbreg G M, Hadrup S R (2015). Design and Validation of Conditional Ligands for HLA-B*08:01, HLA-B*15:01, HLA-B*35:01, and HLA-B*44:05. Cytometry Part A 87A, 967-975
Garboczi D N, Hung D T, Wiley D C (1992). HLA-A2-peptide complexes: refolding and crystallization of molecules expressed in Escherichia coli and complexed with single antigenic peptides. Proc Natl Acad Sci USA 89, 3429-3433.
Jung G, Ledbetter J A, Muller-Eberhard H J (1987). Induction of cytotoxicity in resting human T lymphocytes bound to tumor cells by antibody heteroconjugates. Proc Natl Acad Sci USA 84, 4611-4615.
Luimstra J J, Garstka M A, Roex M C J, Redeker A, Janssen G M C, van Veelen P A, Arens R, Falkenburg J H F, Neefjes J, Ovaa H (2018). A flexible MHC class I multimer loading system for large-scale detection of antigen-specific T cells. J Exp Med. 215(5):1493-1504
Musich J A and Rapoport H. Synthesis of Anthopleurine, the alarm pheromone from anthopleura elegantissima Journal of the American Chemical Society 1978, vol. 100, pp. 4865-4872
Jones J S J (1993). Analysis of polypeptides and proteins. A. Adv. Drug Delivery Rev. 10: 29-90
Nielsen M, Lundegaard C, Blicher T, Lamberth K, Harndahl M, Justesen S, Roder G, Peters B, Sette A, Lund O, Buus S (2007). NetMHCpan, a method for quantitative predictions of peptide binding to any HLA-A and −B locus protein of known sequence. PLoS One 2(8):e796
Ojima I, Wang H, Wang T, and Ng E W (1998). New approaches to the asymmetric synthesis of dipeptide isosteres via beta-lactam synthon method. Tetrahedron Letters vol. 39, pp. 923-926.
Rammensee H, Bachmann J, Emmerich N P, Bachor O A, Stevanović S (1990). Immunogenetics 50: 213-219
Rodenko B, Toebes M, Hadrup S R, van Esch W J, Molenaar A M, Schumacher T N, Ovaa H (2006). Generation of peptide-MHC class I complexes through U V-mediated ligand exchange. Nat Protoc 1, 1120-1132.
Seeger F H, Schirle M, Gatfield J, Arnold D, Keilholz W, Nickolaus P, Rammensee H G, Stevanović S (1999). Immunogenetics 49: 571-576
Thaisrivongs S, Tomasselli A G, Moon J B, Hui J, McQuade T J, Turner S R, Strohbach J W, Howe W J, Tarpley W G, Heinrikson R L (1991). Inhibitors of the protease from human immunodeficiency virus: design and modelling of a compound containing a dihydroxyethylene isostere insert with high binding affinity and effective antiviral activity. Journal of Medicinal Chemistry vol. 34, pp. 2344-2356;
Thaisrivongs S, Turner S R, Strohbach J W, TenBrink R E, Tarpley W G, McQuade T J, Heinrikson R L, Tomasselli A G, Moon J B, Hui J O, Howe W J (1993). Inhibitors of the protease from human immunodeficiency virus: synthesis, enzyme inhibition, and antiviral activity of a series of compounds containing the dihydroxyethylene transition-state isostere. Journal of Medicinal Chemistry, vol. 36, pp. 941-952;
Toebes M, Coccoris M, Bins A, Rodenko B, Gomez R, Nieuwkoop N J, van de K W, Rimmelzwaan G F, Haanen J B, Ovaa H, et al. (2006). Design and use of conditional MHC class I ligands. Nat Med 12, 246-251.

### Items

1. A method for selecting an immune cell expressing on its surface an antigen-binding protein specifically binding to a complex of a peptide A (PA) and a Major Histocompatibility Complex (MHC) molecule, comprising the following steps:
   (i) providing a plurality of immune cells expressing different antigen-binding proteins;
   (ii) contacting the plurality of immune cells with a first composition comprising:
      (a) a complex 1A, comprising an MHC molecule 1 (M1) and a peptide A (PA), and
      (b) a complex 1X, comprising M1 and a peptide B (PB);
   (iii) contacting the plurality of immune cells with a second composition comprising:
      (a) a complex 2A, comprising an MHC molecule 2 (M2) and PA, and
      (b) a complex 2X, comprising M2 and a peptide C (PC);
   (iv) selecting from the plurality of immune cells an immune cell expressing an antigen binding protein that specifically binds to complex 1A; in particular to both complex 1A and complex 2A;
   wherein complexes 1X and 2X, but not 1A and 2A, dissociate upon stimulation with a defined stimulus, preferably a defined chemical and/or physical stimulus; and wherein the amino acid sequences of PB and PC differ in at least one amino acid, preferably in at least 2, 3, 4, 5, 6, 7, or 8 amino acids, more preferably at least 5, 6, 7, or 8 amino acids.
2. The method according to item 1, wherein
   the first composition is obtained by
   (i) providing complex 1X (comprising M1 and PB)
   (ii) providing PA, and
   (iii) providing the defined stimulus, thereby effecting
      dissociation of PB from M1 and
      binding of PA to M1, resulting in formation of complex 1A;
   preferably wherein a composition comprising mainly complex 1A and residual amounts of complex 1X is obtained; and/or
   the second composition are obtained by
   (i) providing complex 2X (comprising M2 and PC)
   (ii) providing PA, and
   (iii) providing the defined stimulus, thereby effecting
   dissociation of PC from M2 and
   binding of PA to M2, resulting in formation of complex 2A;
   preferably wherein a composition comprising mainly complex 2A and residual amounts of complex 2X is obtained.
3. The method according to item 1 or 2, wherein the defined stimulus is a physical and/or chemical stimulus.
4. The method according to any one of items 1 to 3, wherein the defined chemical or physical stimulus is selected from the group consisting of an elevation of temperature, in particular from below 10° C. to 25-45° C., a change of pH, in particular from neutral o acidic or alkaline pH, contacting with periodate, contacting with dithionite, and UV radiation or a combination thereof, preferably from the group consisting of an elevation of temperature, in particular from below 10° C. to 25-45° C., contacting with periodate, contacting with dithionite, and UV radiation or a combination thereof.
5. The method according to any one of items 1 to 4, wherein PB and PC comprise a conditionally reactive group that, when activated by the defined stimulus, effects cleavage of a covalent bond within the peptide backbone of PB or PC, respectively, wherein the conditionally reactive groups comprised in PB and PC may be the same or different, preferably the same.
6. The method according to any one of items 1 to 5, wherein M1 and/or M2, preferably M1 and M2, are MHC-I molecules.
7. The method according to any one of items 1 to 6, wherein M1 and M2 are MHC-I molecules of the same allele or MHC derivatives or antigenic peptide binding fragments of the same allele.
8. The method according to any one of items 1 to 7, wherein M1 and/or M2, preferably M2, are bound to a biotin moiety or a streptavidin moiety, preferably a biotin moiety.
9. The method according to any one of items 1 to 8, wherein M1 and/or M2 are directly or indirectly bound to a carrier, wherein preferably the carrier is a cell, in particular an antigen-presenting cell, or a synthetic carrier, in particular selected from the group consisting of a particle, a filament, a microarray chip and an ELISA plate, more particularly selected from the group consisting of streptavidin, a streptavidin-dextran conjugate, a nanobead and a microbead.
10. The method according to any one of items 2 to 9, wherein the conditionally reactive group is an amino acid analogue present at any position of the amino acid sequence of PB and/or PC except for the first or last position.
11. The method according to any one of items 2 to 10, wherein the conditionally reactive group comprises a light-activatable, dithionite-activatable or periodate-activatable group, preferably a light-activatable group.
12. The method according to item 11, wherein the light-activatable group comprises 3-amino-3-(-2-nitro)phenyl-propionic acid or a light-activatable structural equivalent thereof and/or the periodate sensitive group comprises a dihydroxy moiety.
13. The method according to any one of items 1 to 12, wherein the immune cell is a T cell, more preferably a CD8 T cell.
14. The method according to any one of items 1 to 13, wherein the antigen-binding protein is a T-cell receptor (TCR).
15. The method according to any one of items 1 to 14, wherein the plurality of cells provided in step (i) is obtained from peripheral blood of healthy subjects or subjects that suffer from a disease, or a fraction of the peripheral blood.
16. The method according to item 15, wherein the disease is selected from the group consisting of immune diseases and neoplastic diseases, preferably neoplastic diseases, more preferably cancer.
17. The method according to item 15 or 16, wherein the fraction is enriched in immune cells, preferably T cells, more preferably CD8 T cells or CD4 T cells.
18. The method according to item 17, wherein the immune cell enriched fraction is selected by detectably labeling one or more immune cell specific surface markers, preferably selected from the group consisting of CD3, CD8, CD4 and CD19.
19. The method according to any one of items 1 to 18, wherein the plurality of cells provided in step (i) are T-cells that are phenotyped.
20. The method according to item 19, wherein the phenotyping of T-cells comprises the quantitative or qualitative determination of the presence of one or more T-cell marker, preferably selected from the group consisting of CD3, CD4, CD8, CD11a, CD14, CD19, CD25, CD27, CD28, CD44, CD45RA, CD45RO, CD57, CD62L, CD69, CD122, CD127, CD197 (CCR7), IFNγ, IL-2, TNFα, IL7R and telomer length.
21. The method according to any one of items 1 to 20, wherein step ii) and iii) are performed subsequently, preferably step iii) is performed subsequently to step ii).
22. The method according to any one of items 1 to 21, wherein M1 and/or M2, preferably M2, are bound to a detectable label or to a carrier comprising a detectable label, wherein the detectable labels may be the same or different.
23. The method according to item 21 or 22, wherein the detectable label is selected from the group consisting of a fluorescent label, a magnetic label, an RNA-barcode, a DNA barcode, and a radioactive label.
24. The method according to item 23, wherein the fluorescent label is selected from the group consisting of xanthens, acridines, oxazines, cynines, styryl dyes, coumarines, porphines, metal-ligand-complexes, fluorescent proteins, nanocrystals, perylenes and phtalocyanines, more preferably streptavidin-phycoerythrin (SA-PE), streptavidin-phycoerythrin-cyanine5 (SA-PE-Cy5), streptavidin-allophycocyanin (SA-APC), streptavidin-allophycocyanin-cyanine7 (SA-APC-Cy7), streptavidin-peridinin chlorophyll-A protein (SA-PerCP), streptavidin-peridinin chlorophyll-A protein-cyanine5.5 (SA-PerCP/Cy5.5), streptavidin-phycoerythrin-cyanine7 (SA-PE-Cy7), streptavidin-brilliant-violet 421 (SA-BV421), streptavidin-brilliant-violet 510 (SA-BV510), streptavidin-brilliant-violet 570 (SA-BV570), streptavidin-brilliant-violet 605 (SA-BV605), streptavidin-brilliant-violet 650 (SA-BV650), streptavidin-brilliant-violet 711, streptavidin-brilliant-violet 785SA-BV785, streptavidin VioBlue (SAVioBlue), streptavidin allophycocyanin Vio770 (SA APC Vio770), streptavidin peridinin chlorophyll-A protein Vio700 (SA PerCPVio700), streptavidin phycoerythrin Vio 770 (SA PE Vio770) and streptavidin-phycoerythrin-Dazzle594.
25. The method according to any one of items 23 to 24, wherein the label is detectable by flow cytometry analysis, preferably fluorescence activated cell sorting (FACS) or microfluidic analysis or preparative sorting analysis like magnetic activated cell sorting (MACS).
26. The method according to any one of items 1 to 25, wherein the cell selected in step iv) is a single cell or a population of cells.
27. The method according to any one of items 1 to 26, wherein step ii)
   (i) further comprises contacting the plurality of cells with an antibody against a costimulatory molecule, preferably anti-CD28, anti-CD3, anti-CD137 and/or anti-CD134, more preferably anti-CD28 and/or anti-CD3;
   (ii) further comprises contacting the plurality of cells with TL-2 and/or TL-12; and/or (iii) is carried out for at least 3 days, preferably at least 7 days, more preferably at least 10 days.
28. The method according to any one of items 1 to 27, wherein step iii) further comprises contacting the plurality of cells with a viability dye and/or a labeled surface marker; preferably CD3, CD4, CD8 and/or CD69; more preferably CD3 and/or CD8.
29. The method according to any one of items 1 to 28, wherein step iv) comprises at least one of detection, characterization and isolation and optionally cultivation of a single cell or population of cells.
30. The method according to any one of items 1 to 29, wherein PA is an antigenic peptide, preferably selected from a viral antigenic peptide, a bacterial antigenic peptide and a tumor associated antigenic peptide (TAA), more preferably PA is a TAA.
31. The method according to any one of items 1 to 30, wherein PA, PB and/or PC
   are MHC-I binding peptides; and/or
   have a length of 8-12 amino acids, preferably 8-11 amino acids.
32. The method according to any one of items 1 to 31, wherein PB and/or PC
   comprise the conditionally reactive group at a position that does not require a certain amino acid for MHC binding;
   comprise the conditionally reactive group at a solvent exposed position;
   have a relative Syfpeithi score of at least 0.4, preferably at least 0.5, more preferably at least 0.6, even more preferably at least 0.7; and/or
   have a NetMHCpan rank of 0.2 or less, 0.1 or less, 0.05 or less, 0.02 or less, 0.01 or less, preferably 0.005 or less.
33. The method according to any one of items 2 to 32, wherein PB and/or PC together with an MHC molecule form a complex 1X and/or 2X, respectively, wherein 1X and/or 2X
   have a refolding yield of at least 0.5, at least 0.2, at least 0.1, at least 0.05, preferably at least 0.025 mol pHLA per mol HLA chain;
   are stable for at least 1 hour, at least 2 hours, at least 6 hours, at least 12 hours, at least 18 hours, or at least 24 hours at room temperature in a buffer, such as PBS, in the absence of UV exposure;
   have a low aggregation;
   have a low degradation rate; and/or
   have a dissociation rate after cleavage of about >50%, preferably about >70%, more preferably about >90%.
34. The method according to any one of items 1 to 33, wherein PB and/or PC are derived from peptides that are frequently presented in individuals expressing the MHC allele on which PB and PC are presented.
35. The method according to any one of items 1 to 34, wherein the amino acid sequence of PB and PC differ in at least one amino acid and wherein
   i) M1 and M2 is HLA-A*01:01 or a MHC derivative thereof or a antigenic peptide binding fragment thereof, and the amino acid sequences of PB and PC are selected from the group consisting of MUC16-010, MUC16-011, RNF213-010, RNF213-011, AXIN1-003, AXIN1-004, CLTC-002, CLTC-003, CLTC-004, preferably CLTC-002 and MUC16-010, more preferably PB is CLTC-002 and PC is MUC16-010, wherein MUC16-010 comprises or consists of SEQ ID NO: 1, MUC16-011 comprises or consists of SEQ ID NO: 2, RNF213-010 comprises or consists of SEQ ID NO: 3, RNF213-011 comprises or consists of SEQ ID NO: 4, AXIN1-003 comprises or consists of SEQ ID NO: 5, AXIN1-004 comprises or consists of SEQ ID NO: 6, CLTC-002 comprises or consists of SEQ ID NO: 7, CLTC-003 comprises or consists of SEQ ID NO: 8, and CLTC-004 comprises or consists of SEQ ID NO: 9; or
   ii) M1 and M2 is HLA-A*02:01 or a MHC derivative thereof or a antigenic peptide binding fragment thereof, and the amino acid sequences of PB and PC are selected from the group consisting of DDX5-002, DDX5-003, DDX5-004, DDX5-005, DDX5-006, RPL19-003, RPL19-004, RPL19-005, and FLUM-003, preferably DDX5-002, DDX5-003, DDX5-004, DDX5-005, DDX5-006, RPL19-003, RPL19-004, RPL19-005, more preferably DDX5-005 and RPL19-005, even more preferably PB is DDX5-005 and PC is RPL19-005, wherein DDX5-002 comprises or consists of SEQ ID NO: 10, DDX5-003 comprises or consists of SEQ ID NO: 11, DDX5-004 comprises or consists of SEQ ID NO: 12, DDX5-005 comprises or consists of SEQ ID NO: 13, DDX5-006 comprises or consists of SEQ ID NO: 14; RPL19-002 comprises or consists of SEQ ID NO: 16, RPL19-003 comprises or consists of SEQ ID NO: 17, RPL19-002 comprises or consists of SEQ ID NO: 18; FLUM-003 comprises or consists of SEQ ID NO: 19; or
   iii) M1 and M2 is HLA-A*03:01 or a MHC derivative thereof or a antigenic peptide binding fragment thereof, and the amino acid sequences of PB and PC are selected from the group consisting of UVLA3-001, GIBB-002, GIBB-003, HNRNPR-002, HNRNPR-003 and HNRNPR-004, preferably GIBB-002 and HNRNPR-002, more preferably PB is GIBB-002 and PC is HNRNPR-002, wherein UVLA3-001 comprises or consists of SEQ ID NO: 20, GIBB-002 comprises or consists of SEQ ID NO: 21, GIBB-003 comprises or consists of SEQ ID NO: 22, HNRNPR-002 comprises or consists of SEQ ID NO: 24, HNRNPR-003 comprises or consists of SEQ ID NO: 25, HNRNPR-004 comprises or consists of SEQ ID NO: 26; or
   iv) M1 and M2 is HLA-A*24:02 or a MHC derivative thereof or a antigenic peptide binding fragment thereof, and the amino acid sequences of PB and PC are selected from the group consisting of TEG-002, TEG-003, TEG-004, TEG-005, TEG-006, TEG-007, PPP1CA-002, PPP1CA-003, PPP1CA-005, preferably TEG-007 and PPP1CA-005, more preferably PB is TEG-007 and PC is PPP1CA-005, wherein TEG-002 comprises or consists of SEQ ID NO: 29, TEG-003 comprises or consists of SEQ ID NO: 30, TEG-004 comprises or consists of SEQ ID NO: 31, TEG-005 comprises or consists of SEQ ID NO: 32, TEG-006 comprises or consists of SEQ ID NO: 33, TEG-007 comprises or consists of SEQ ID NO: 34, PPP1CA-002 comprises or consists of SEQ ID NO: 35, PPP1CA-003 comprises or consists of SEQ ID NO: 36, PPP1CA-005 comprises or consists of SEQ ID NO: 38; or
   v) M1 and M2 is HLA-B*07:02 or a MHC derivative thereof or a antigenic peptide binding fragment thereof, and the amino acid sequences of the PB and PC are different and selected from the group consisting of UVLB7-001, HLA-DPB1-002, HLA-DPB1-003, HLA-DPB1-004, CHCHD2-003, and MUDENG-002, preferably MUDENG-002 and DPB1-004, more preferably PB is MUDENG-002 and PC is DPB1-004, wherein UVLB7-001 comprises or consists of SEQ ID NO: 39, HLA-DPB1-002 comprises or consists of SEQ ID NO: 40, HLA-DPB1-003 comprises or consists of SEQ ID NO: 41, HLA-DPB1-004 comprises or consists of SEQ ID NO: 42, CHCHD2-003 comprises or consists of SEQ ID NO: 44, and MUDENG-002 comprises or consists of SEQ ID NO: 46; or
   vi) M1 and M2 is HLA-B*08:01 or a MHC derivative thereof or a antigenic peptide binding fragment thereof, and the amino acid sequences of PB and PC are selected from the group consisting of MKI-003, MKI-004, VIM-004, VIM-005, COPA-008, PDCD10-002, PDCD10-003, MIR286-002, EBV-020, SPTAN-002 and LOC646214-003, preferably MKI-003, COPA-008, PDCD10-003, MIR286-002, EBV-020, SPTAN-002 and LOC646214-003, more preferably SPTAN-002 and LOC646214-003, even more preferably PB is SPTAN-002 and PC is LOC646214-003, wherein MKI-003 comprises or consists of SEQ ID NO: 49, MKI-004 comprises or consists of SEQ ID NO: 50, VIM-004 comprises or consists of SEQ ID NO: 51, VIM-005 comprises or consists of SEQ ID NO: 52, COPA-008, comprises or consists of SEQ ID NO: 53, PDCD10-002 comprises or consists of SEQ ID NO: 54, PDCD10-003 comprises or consists of SEQ ID NO: 55, MIR286-002 comprises or consists of SEQ ID NO: 56, EBV-020 comprises or consists of SEQ ID NO: 57, SPTAN-002 comprises or consists of SEQ ID NO: 58, LOC646214-003 comprises or consists of SEQ ID NO: 59;
   vii) M1 and M2 is HLA-B*44:02 or a MHC derivative thereof or a antigenic peptide binding fragment thereof, and the amino acid sequences of PB and PC are selected from the group consisting of SPTBN1-002, SPTBN1-003, SPTBN1-004, CLTC-006, CLTC-007, CSE1-005 and CSE1-006, preferably SPTBN1-004 and CSE1-005, more preferably PB is SPTBN1-004 and PC is CSE1-005, wherein SPTBN1-002 comprises or consists of the amino acid sequence SEQ ID NO: 60, SPTBN1-003 comprises or consists of SEQ ID NO: 61, SPTBN1-004 comprises or consists of SEQ ID NO: 62, CLTC-006 comprises or consists of SEQ ID NO: 63, CLTC-007 comprises or consists of SEQ ID NO: 64, CSE1-005 comprises or consists of SEQ ID NO: 65 and CSE1-006 comprises or consists of SEQ ID NO: 66; or
   viii) M1 and M2 is HLA-B*44:05 or a MHC derivative thereof or a antigenic peptide binding fragment thereof, and the amino acid sequences of PB and PC are selected from the group consisting of SPTBN1-002, SPTBN1-003, SPTBN1-004, CLTC-006, CLTC-007, CSE1-005 and CSE1-006, preferably SPTBN1-004 and CLTC-006, more preferably PB is SPTBN1-004 and PC is CLTC-006, wherein SPTBN1-002 comprises or consists of the amino acid sequence SEQ ID NO: 60, SPTBN1-003 comprises or consists of SEQ ID NO: 61, SPTBN1-004 comprises or consists of SEQ ID NO: 62, CLTC-006 comprises or consists of SEQ ID NO: 63, CLTC-007 comprises or consists of SEQ ID NO: 64, CSE1-005 comprises or consists of SEQ ID NO: 65 and CSE1-006 comprises or consists of SEQ ID NO: 66,
   wherein in SEQ ID NO: 1 to SEQ ID NO: 66 the X represents a conditionally reactive group.
36. A conditional peptide having an amino acid sequence selected from the group consisting of SEQ ID NO: 1-19, 21-38 and 40-66, wherein in wherein in each sequence the X represents a conditionally reactive group, preferably a conditionally reactive amino acid analogue.
37. The conditional peptide according to item 36, wherein the conditional peptide is derived from a parental peptide that is frequently presented in individuals expressing the MHC allele on which said peptide is presented.
38. The conditional peptide according to item 36 or 37, wherein a conditional peptideMHC complex comprising said conditional peptide
   has a refolding yield of at least 0.5, at least 0.2, at least 0.1, at least 0.05, preferably at least 0.025 mol pHLA per mol HLA chain;
   is stable for at least 1 hour, at least 2 hours, at least 6 hours, at least 12 hours, at least 18 hours, or at least 24 hours at room temperature in a buffer, such as PBS, in the absence of UV exposure;
   has a low aggregation;
   has a low degradation rate; and/or
   has a dissociation rate after cleavage of about >50%, preferably about >70%, more preferably about >90%.
39. The conditional peptide according to any one of items 36 to 38, wherein the conditional peptide is selected from the group consisting of SEQ ID NO: 1-18, 21-38, 40-56 and 58-66, preferably selected from the group consisting of SEQ ID NO: 3-18, 21-38, 40-48, 51-56, and 59-66.
40. The conditional peptide according to any one of items 36 to 39, wherein the conditional peptide is specific for HLA-A*01:01, HLA-A*02:01, HLA-A*03:01, HLA-A*24:02, HLA-B*07:02, HLA-B*07:02, HLA-B*08:01 or HLA-B*44:05.
41. An immune cell selected by the method of any one of items 1 to 35.
42. A method for determining the sequence of a nucleic acid encoding an antigen-binding protein specifically binding to a pMHC complex 1A comprising the steps of:
   (i) isolating a nucleic acid encoding the antigen-binding protein from the immune cell selected in the method of items 1 to 35; and
   (ii) determining the sequence of the nucleic acid.
43. A method for producing a host cell expressing an antigen-binding protein comprising the steps of:
   (i) providing a host cell;
   (ii) isolating a nucleic acid encoding the antigen-binding protein from the immune cell selected in the method of items 1 to 35;
   (iii) producing a genetic construct comprising the nucleic acid isolated in step (ii) or a part of said nucleic acid encoding the antigen-binding protein or an antigen-binding fragment thereof; and
   (iv) introducing the genetic construct into the host cell.
44. A method for producing an antigen-binding protein comprising providing a host cell produced by the method of item 43 and expressing the genetic construct introduced into said host cell.
45. The methods according to items 43 or 44, wherein the antigen-binding protein is a TCR or antigen binding fragment thereof, wherein preferably the TCR or antigen binding fragment thereof comprises six CDR sequences.
46. An antigen binding protein produced by the method of item 44.
47. A nucleic acid encoding the antigen binding protein of item 46 or a vector comprising said nucleic acid.
48. A kit, preferably for selecting a cell expressing on its surface an antigen-binding protein specifically binding to a complex of a peptide and a MHC molecule, wherein said kit comprises:
   a) a complex 1X, comprising a peptide B (PB) and an MHC molecule 1 (M1); and a complex 2X, comprising a peptide C (PC) and an MHC molecule 2 (M2); or
   b) a peptide (PB) and a peptide (PC) and optionally (an) MHC molecule(s) to which PB and PC bind, in particular M1 and/or M2, thereby forming a complex 1X and 2X, respectively; and c) optionally β2-microglobulin;
   wherein complexes 1X and 2X, dissociate upon stimulation with a defined chemical and/or physical stimulus; and wherein the amino acid sequences of PB and PC differ in at least one amino acid, preferably in at least 2, 3, 4, 5, 6, 7, or 8 amino acids, for selecting an antigen binding protein specifically binding to a complex 1A comprising a peptide A (PA) and M1 or M2.
49. A pharmaceutical composition comprising an immune cell selected by the method of any one of items 1 to 35, a host cell produced by the method of item 43, an antigen binding protein of item 46 and/or a nucleic acid or vector of item 47.
50. An immune cell selected by the method of any one of items 1 to 35, a host cell produced by the method of item 40, an antigen binding protein of item 44 and/or a nucleic acid or vector of item 45 for use in medicine.
51. An immune cell selected by the method of any one of items 1 to 35, a host cell produced by the method of item 43, an antigen binding protein of item 46 and/or a nucleic acid or vector of item 47 for use in a method of treatment or prevention of a neoplastic disease.
52. A method for treating a neoplastic disease comprising administration of a therapeutically effective amount of an immune cell selected by the method of any one of items 1 to 35, a host cell produced by the method of item 43, an antigen binding protein of item 46 and/or a nucleic acid or vector of item 47 to a subject in need thereof.
53. A method for treating a neoplastic disease in a subject in need thereof comprising the steps of:
   (i) providing an immune cell population of the subject comprising T cells;
   (ii) contacting the immune cell population of step (i) with a first composition comprising:
      (a) a complex 1A, comprising a MHC molecule 1 (M1) and a peptide A (PA), and
      (b) a complex 1X, comprising M1 and a peptide B (PB);
   (iii) contacting the immune cell population with a second composition comprising:
      (a) a complex 2A, comprising a MHC molecule 2 (M2) and PA, and
      (b) a complex 2X, comprising M2 and a peptide C (PC);
   (iv) selecting from the immune cell population at least one T cell expressing a TCR that specifically binds to complex 1A; in particular to both complex 1A and complex 2A; and
   (v) increasing the number of the at least one selected T cell by cultivation; and
   (vi) reintroducing the cultivated T cells into the subject;
   wherein complexes 1X and 2X, but not 1A and 2A, dissociate upon stimulation with a defined stimulus; and wherein the amino acid sequences of PB and PC differ in at least one amino acid.
54. A use of a peptide according to any one of items 36 to 39 for the preparation of pMHC complexes.

1. A method for selecting an immune cell expressing on its surface an antigen-binding protein specifically binding to a complex of a peptide A (PA) and a Major Histocompatibility Complex (MHC) molecule, comprising the following steps:
(i) providing a plurality of immune cells expressing different antigen-binding proteins;
(ii) contacting the plurality of immune cells with a first composition comprising:
(a) a complex 1A, comprising an MHC molecule 1 (M1) and a peptide A (PA), and
(b) a complex 1X, comprising M1 and a peptide B (PB);
(iii) contacting the plurality of immune cells with a second composition comprising:
(a) a complex 2A, comprising an MHC molecule 2 (M2) and PA, and
(b) a complex 2X, comprising M2 and a peptide C (PC);
(iv) selecting from the plurality of immune cells an immune cell expressing an antigen binding protein that specifically binds to complex 1A; optionally to both complex 1A and complex 2A;
wherein complexes 1X and 2X, but not 1A and 2A, dissociate upon stimulation with a defined stimulus, optionally a defined chemical and/or physical stimulus; and
wherein the amino acid sequences of PB and PC differ in at least one amino acid, optionally in at least 2, 3, 4, 5, 6, 7, or 8 amino acids, optionally at least 5, 6, 7, or 8 amino acids.

2. The method according to claim 1, wherein
the first composition is obtained by
(i) providing complex 1X (comprising M1 and PB)
(ii) providing PA, and
(iii) providing the defined stimulus, thereby effecting dissociation of PB from M1 and
binding of PA to M1, resulting in formation of complex 1A;
optionally wherein a composition comprising mainly complex 1A and residual amounts of complex 1X is obtained; and/or
the second composition is obtained by
(i) providing complex 2X (comprising M2 and PC)
(ii) providing PA, and
(iii) providing the defined stimulus, thereby effecting
dissociation of PC from M2 and
binding of PA to M2, resulting in formation of complex 2A;
optionally wherein a composition comprising mainly complex 2A and residual amounts of complex 2X is obtained.

3. The method according to claim 1, wherein PB and PC comprise a conditionally reactive group that, when activated by the defined stimulus, effects cleavage of a covalent bond within the peptide backbone of PB or PC, respectively,
wherein the conditionally reactive groups comprised in PB and PC may be the same or different, optionally the same, and
wherein optionally the conditionally reactive group comprises a light-activatable, dithionite-activatable or periodate-activatable group, optionally a light-activatable group, more optionally a light-activatable group comprises 3-amino-3-2-nitro)phenyl-propionic acid or a light-activatable structural equivalent thereof.

4. The method according to claim 1, wherein M1 and/or M2, optionally M1 and M2, are MHC-I molecules, optionally MHC-I molecules of the same allele or MHC derivatives or antigenic peptide binding fragments of the same allele.

5. The method according to claim 1, wherein the immune cell is a T cell, optionally a CD 8 T cell and/or wherein the antigen-binding protein is a T-cell receptor (TCR).

6. The method according to claim 1, wherein step ii)
(i) further comprises contacting the plurality of cells with an antibody against a costimulatory molecule, currently anti-CD28, anti-CD3, anti-CD137 and/or anti-CD134, optionally anti-CD28 and/or anti-CD3;
(ii) further comprises contacting the plurality of cells with IL-2 and/or IL-12; and/or
(iii) is carried out for at least 3 days, optioionally at least 7 days optionally at least 10 days; and/or
wherein step iv) comprises at least one of detection, characterization and isolation and optionally cultivation of a single cell or population of cells.

7. The method according to claim 1, wherein PA is an antigenic peptide, optionally selected from a viral antigenic peptide, a bacterial antigenic peptide and a tumor associated antigenic peptide (TAA), prefer-optionally PA is a TAA.

8. A conditional peptide having an amino acid sequence selected from the group consisting of SEQ ID NO: 1-19, 21-38 and 40-66, wherein in SEQ ID NO: 1 to SEQ ID NO. 66 the X represents a conditionally reactive group, optionally a conditionally reactive amino acid analogue.

9. The conditional peptide according to claim 8, wherein the conditional peptide is derived from a parental peptide that is frequently presented in individuals expressing the MHC allele on which said peptide is presented, optionally wherein the conditional peptide is selected from the group consisting of SEQ ID NO: 1-18, 21-38, 40-56 and 58-66, optionally selected from the group consisting of SEQ ID NO: 3-18, 21-38, 40-48, 51-56, and 59-66.

10. An immune cell selected by the method of claim 1.

11. A method for determining the sequence of a nucleic acid encoding an antigen-binding protein specifically binding to a pMHC complex 1A comprising the steps of:
(i) isolating a nucleic acid encoding the antigen-binding protein from the immune cell selected in the method of claim 1; and
(ii) determining the sequence of the nucleic acid.

12. A method for producing a host cell expressing an antigen-binding protein comprising the steps of:
(i) providing a host cell;
(ii) isolating a nucleic acid encoding the antigen-binding protein from the immune cell selected in the method of claim 1;
(iii) producing a genetic construct comprising the nucleic acid isolated in step (ii) or a part of said nucleic acid encoding the antigen-binding protein or an antigen-binding fragment thereof; and
(iv) introducing the genetic construct into the host cell.

13. A method for producing an antigen-binding protein comprising providing a host cell produced by the method of claim 12 and expressing the genetic construct introduced into said host cell.

14. A use of a peptide according to claim 8 for the preparation of pMHC complexes.

15. A kit, optionally for selecting a cell expressing on its surface an antigen-binding protein specifically binding to a complex of a peptide and a M-IC molecule, wherein said kit comprises:
a) a complex 1X, comprising a peptide B (PB) and an MHC molecule 1 (M1); and a complex 2X, comprising a peptide C (PC) and an MHC molecule 2 (M2); or
b) a peptide (PB) and a peptide (PC) and optionally (an) MHC molecule(s) to which PB and PC bind, in optionally M1 and/or M2, thereby forming a complex 1X and 2X, respectively; and c) optionally p2-microglobulin;
wherein complexes 1X and 2X, dissociate upon stimulation with a defined chemical and/or physical stimulus; and wherein the amino acid sequences of PB and PC differ in at least one amino acid, optionally in at least 2, 3, 4, 5, 6, 7, or 8 amino acids, for selecting an antigen binding protein specifically binding to a complex 1A comprising a peptide A (PA) and M1 or M2.

16. A pharmaceutical composition comprising an immune cell selected by the method of claim 1, a host cell, and/or an antigen binding protein, a nucleic acid and/or vector.

17. An immune cell selected by the method of claim 1, a host cell, and/or an antigen binding protein, a nucleic acid and/or vector for use in medicine, optionally for use in a method of treatment or prevention of a neoplastic disease.

18. An antigen binding protein produced by the method of claim 13, a nucleic acid encoding said antigen binding protein or a vector comprising said nucleic acid.

19. The method according to claim 2, wherein PB and PC comprise a conditionally reactive group that, when activated by the defined stimulus, effects cleavage of a covalent bond within the peptide backbone of PB or PC, respectively,
wherein the conditionally reactive groups comprised in PB and PC may be the same or different, optionally the same, and
wherein optionally the conditionally reactive group comprises a light-activatable, dithionite-activatable or periodate-activatable group, optionally a light-activatable group, optionally a light-activatable group comprises 3-amino-3-(-2-nitro)phenyl-propionic acid or a light-activatable structural equivalent thereof.

20. The method according to claim 2, wherein M1 and/or M2, optionally M1 and M2, are MHC-I molecules, optionally MHC-I molecules of the same allele or MHC derivatives or antigenic peptide binding fragments of the same allele.
